SEQUENCE LISTING (45 sequences)

SEQ ID NO: 1 - DNA, 20370 bp, Homo sapiens
atcatgccctgaacctcatgagcccaggcaggcatcctgttggttaaggtcggcctcagggtccagggatgcactagattctttatcttaaagtgaccctcaagaaaaaaaaaataaaatgtagctataggcagtttctggacgttttcctatttgttaagcaccttcttgctccgagctttccagccacagtgcactggacgccatcggaaggctgcagctgctatttggtgctttaccaccgctgagaaggtggaatggtcacggcccggttctgtcactttcttcagccggacagtcgccttattacggattccagtagggccgagcacactccttcctgcccgtctttacagatgaacatgctatcgctccagcagtacaacccgccttattacgtaaaaaagcagcccctatctacccgcagggaaggagtatgttcggcaccacagctggactggtgctcgagttaagcgtcctgggaggtccgcatgcgctccggaaccgtaatgcgcgctttttctaagccttacggtaaacgcggacgcagggcaaccacgtggcggtggaaccgaggcccggcgggaatgcgcggaatgcgcggcgcggcctcgcgcggttcccgagccacggcccagggtccggcggcgcgcgctctcgcctcctcccctcacctctcccagccgcaccccggccctggccctgccgcccagaactcgctgggcaagtcgtgccccgcgtgaacacacagaaggggcttggggaccgagcgcggcccatcagtccctcagaccctgaggacccagaattccctaaggggtccgaatccgagtcctgcccccagcccttaaggcacgggctccagggaccccaggggaagggcgcggggcattaggtacgcaacccgtttccccgcacctggaaaaaaactccctttccctcccctcccctgcttgttgagtgtccggataaccagaactctaaggcgccccgtaataacgaccccgctgtccctccacccacccccaagtgccaaagcgagggatggaagcgctttcaagcgttccaagggcattgaggagcgagctggagaggcgcggggatgcggggtcctccccgcagtcttccggaaagggcgggggagggcgcggcaagttccggagtggggcatgccgtgggagcccacgagggcctcagcgcggatcctccgccggaaaaccggctcccgcgagccgccgccgcaggtttcctaggccccgcgagtcccgcagcgaagccctgcgtctccgtccgacgcgggggtctgctcagcctcgggtgggccgcggccaggcctgactgcgggggagagggccgaacgtgacctccgaggtcacccccagccagctttctctcctgtggtcggaagtggttttcttctcgatctgggcgcctactccccaccacttggtctgagaggggctggggccggaaggccagggaatctctggtggatttgggggttcatattgctcagggtaccagccgatgcgttttgaggggcgggagtcgaggaattagaatcgcctttaaccctcaagagttgcgccttcagcctcgggatcccagatgcgtcgttggagccagggccgcccccctacctgttgggtttgcgttttaactccagcgcacaccttgccggcagccctcggagctaggggaggggtctcgtttccccgcagcccgccggacagacgactggggcacgggaggggcggtggcagggtggtctgtgtgtggctgaaactaattgatctggagcggaaacgcacgtctgcggttggggcgatggggggggcggtgcggctgtccatgtgccgagcgtgtggctgtctcgggtgggcactggggccggagttcgccccggcccacctcgcagttttggggcgcctgggatcggcgctacgtaagcgaagcagagctgccatagcacgtgggccgccacgcgcaccccaaaagcaagcagtgtggggggaaggggagctcgagcgccttcggagcccaggggccggctttcggaagcgttttcccgggcgacttaagggcttaacaatggaaaactcgcggagcctgagccaagtcctttcaagtcgccgccaggtatgcggctgcaggtgaccccacctgggtgcgcccgcccgccagccgccctggtggaaaagcgggtgcgggaggtcgctggcgaaaggtcgggactggtccctgcaccacccgcccccaacccaagccccgagccccgcggcgcgcagccgcgctgagtcccggggtctgcgtcgcggcgcgccggttcctgaatgaacgcgctcccttcccccgcctgaatgaaggttcccacagccagggacggtggcgaacacgcgcctgcagcggaattcgctttctcctgaccgaccatccgcccaggccgcggtcaccggggcgggggccagggggcgaggaaagcgtgaaggtgatttcagttaattttggattttctttcaaacaacgtggttaccctcccgactgggccacttgccctttgtctccaaatggtcaccaagaaataagaacagagcactttaaatgagcccagaatccgcagttcctgcttcgtggtgggttttaagaagacagtgtaaagtaaaactgcaaccgaaaagttttttaaagttgcttttctctttggaaaaaataaaatcaaaatgctttctctgcgcttcttgaagcaatgaccctcaaaagcccagaggtattggccccctcgggggacccgggggccgccaagcagggttcccccaggtgggggctgggcagctggcgctccccgccgggccccaaattccagcgccgggccccaaattccagcgcctcccccgcgggttcctggacggctctttacgctcgctaaccgggcttgcaattttgcgctcgtccctgagccgggaaatcaacgaagttcctagtcgagatctgcccggtccgcctagtaacagcgccgcgcccccattggctcatgctaattccagtttcctctgtcttgcgcccgggatgggggggtgaagctccctcctggacccagagccggttgtgccggagtgggcgagcctctttatgccctgctgcccctagccgacttcggcccgcttcgcgcctcgggctgggccagggcgcacgcggggctcggggcccctcgccccacgggatgggagaggccgggtgatagctccgggccccataaatcatccaggcggccgccgggtcgggattttatgaatgaaaaagcagctgggccgcccttgtgcgcgggctgatgctctgaggcttggctatgcgggggccaacgcgattgtgggtgctcggggagtgggggggggcacgaccgtaggtgctccctgctggggcaacccatcgctccccatgcggaatccgggggtaattacccccccaggacccggaatattagtaatcctaattcccggcgggggagggggcgcgggaggaattcaccctgaaaggtgggggtggggggggtcgcatcttgctgtgagcaccctggcgaaggggagagggctttttctatcagttttctttgagcttttactgttaagagggtacggtggtttgatgacactgaactatattcaaaaggaagtaaatgaacagttttcttaatttggggcaggtactgtaaaaataaaaacaaaagttaagacagtaaaatgtccttttattttttaatgcaccaaagagacagaacctgtaattttaaaaactgtgtattttaatttacatctgcttaagtttgcgataatattggggaccctctcatgtaaccacgaacacctatcgattttgctaaaaatcagatcagtacactcgtttgtttaattgataattgttctgaattatgccggctcctgccagccccctcacgctcacgaattcagtcccagggcaaattctaaaggtgaagggacgtctacacccccaacaaaaccaattaggaaccttcggtggtcttgtcccaggcagaggggactaatatttccagcaatttaatttcttttttaattaaaaaaaatgagtcagaatggagatcactgtttctcagctttccattcagaggtgtgtttctcccggttaaattgccggcacgggaagggagggggtgcagttggggacccccgcaaggaccgactggtcaaggtaggaaggcagcccgaagagtctccaggctagaaggacaagatgaaggaaatgctggccaccatcttgggctgctgctggaattttcgggcatttattttattttattttttgagcgagcgcatgctaagctgaaatccctttaacttttagggttacccccttgggcatttgcaacgacgcccctgtgcgccggaatgaaacttgcacaggggttgtgtgcccggtcctccccgtccttgcatgctaaattagttcttgcaatttacacgtgttaatgaaaatgaaagaagatgcagtcgctgagattctttggccgtctgtccgcccgtgggtgccctcgtggcgttcttggaaatgcgcccattctgccggcttggatatggggtgtcgccgcgccccagtcaccccttctcgtggtctccccaggctgcgtgtggcctgccggccttcctagttgtcccctactgcagagccacctccacctcaccccctaaatcccgggggacccactcgaggcggacggggccccctgcacccctcttccctggcggggagaaaggctgcagcggggcgatttgcatttctatgaaaaccggactacaggggcaactccgccgcagggcaggcgcggcgcctcagggatggcttttgggctctgcccctcgctgctcccggcgtttggcgcccgcgccccctccccctgcgcccgcccccgcccccctcccgctcccattctctgccgggctttgatctttgcttaacaacagtaacgtcacacggactacaggggagttttgttgaagttgcaaagtcctggagcctccagagggctgtcggcgcagtagcagcgagcagcagagtccgcacgctccggcgaggggcagaagagcgcgagggagcgcggggcagcagaagcgagagccgagcgcggacccagccaggacccacagccctccccagctgcccaggaagagccccagccatggaacaccagctcctgtgctgcgaagtggaaaccatccgccgcgcgtaccccgatgccaacctcctcaacgaccgggtgctgcgggccatgctgaaggcggaggagacctgcgcgccctcggtgtcctacttcaaatgtgtgcagaaggaggtcctgccgtccatgcggaagatcgtcgccacctggatgctggaggtgcggggcttcgggcggctctcttaagacttccctgcaacttgttgcccagacccacgtttctttgctactcacccccctcccttctctcccgctagaactttgaagtttgccgtggtgtttctagggatccgtattttcaaaataaaaattgcgggtattttctgaaggaggaaggggtgggggtgggggtgctagaagtagcgtttcgtgggaggggagaagggggtccgggaggggtgccttcgggagaagccagtgccaggggcaccccaatgggcccgagggtgcgggctggcaggctgggtgcgctttgtgtcccccgcctgcgccccagcccggctgcgcctcagcggccgggagccgccaactccggggggagggggcatagatttgatttttaaattaatatccatggacacgtatgcaagggccgctcgtgccagtattatgcgccatctttgctcttttattgcaaagcaaaagtgtttattaataattgggggcagggtgggggcggggagcggccgccgggcgctggggccgcagctaagggccgcgcggctgccgggagcccgcgggaggggcgcagggacgcggcatgggtagttttggggggacgccgctagggaagggggggcctttgttcaagcagcgagtcccggggcgccccgaacgggcagcctgggccggagagcacggcgagctgcaaggtcgcgtggcccccaagacgccagggcttgatccccgtctgcagggatatcggcttggaggaccttctccgagcgagccgggggcctgggagcacattttcagaccttcggtgggcgcctgaggggcccgcaagtattttaaaataatttttgaaagtgcggcgtggtgcccttgcgagagggaaacgccgcccgcgcccagggggaagggggggccccggagtttgaattcctggggctccccccggagcctgtaacgaactcccaacccccggcctgggtaaagggtcgcccgagggtcattttcagggtttttttatgcacttagttatttttttaatatttttaaatattttttgaaaagatgacgtctggggaaatgcggcgcggcggcctgggacgccacctttgtgtctcgcaggcgcggcgcccaaccccgcggcccgttccgcggccccgcaccccagttggtgtcgacccccagtcagagggaccacggagctccagggcgggccagggtcccgggggccggcagcccgcgccgccgcgcacgccgcccagctgtgcccgctcccgcccccaccgtgccagcctcgcggggactttccctttcagtttcggggagggtgggtactggggacgcgcgggggagggggcgcatcacgggaagctcctgccgcccccagccccgacccctcggcgccctccagacctggcggccctgccaagcgcgatggggggtgcgggggcgtgcgggggggcggcgcgacctggcggcggcggtcacgggccccgtgcctccgtaggtctgcgaggaacagaagtgcgaggaggaggtcttcccgctggccatgaactacctggaccgcttcctgtcgctggagcccgtgaaaaagagccgcctgcagctgctgggggccacttgcatgttcgtggcctctaagatgaaggagaccatccccctgacggccgagaagctgtgcatctacaccgacaactccatccggcccgaggagctgctggtaaccactggaccccgccgccccccgccccccgcgagccgcacgcaggaccacggggccggggaaggtgcaggcggtggcggccggcccgcctctgacatatctgctcctccgagggagggcggccccgccgccgggcgtccctgtccggggagcgggcgggatcctagccgccctcgtcccgccgccctgtgtgcgcttgcctgcgactcccaccgcgttcgcgccccgcggtgtggccgaaaagtgggcggcgcgcgccctccagcggctgcacgaggagcgccgcgctcggcgctgagcctccagttccaggtggtgggaggtctttttgtttccacttgcagagtcttttcacgcggcgggcgccttttctgttttgatctgggattgcgtgttgccccagctcccttgagtccccagcattcgccagccctcccctccaacatccaggaccgcacgagacgcaggggccagtgctctgagccggaggtgcggcgtggcccggcccccgtgctgccggcttccccgcgcccccgggctggcccgcacctcccctgatggccgctcaccctgtgttcgcagcaaatggagctgctcctggtgaacaagctcaagtggaacctggccgcaatgaccccgcacgatttcattgaacacttcctctccaaaatgccagaggcggaggagaacaaacagatcatccgcaaacacgcgcagaccttcgttgccctctgtgccacaggtagggcaggcccggcagcccccggcctccccttgagagccggctccttaggtgaccctggccggcttcttgctctccacctgggtgctgtctgggaagatgtccccagaccccctcctgcgctggagagcgctcttccagctctggtgagcagaggccctggattgtttgtcgcgctggatggagggagatttgctccctcacggccaccatgcagtaccttgggcattggtgtggacggctcagcctgcctgtgtcccgttactctggcctcgtccttcaggccaggcagcctgtggccactccatgctgaaaggggtttaccttggccacagggccgcctcctttctccacccacctccagcccttcttgtgtccttaaggagcctgagctgcagaggccccctcctggcctctcccaggctgggccacctgccagaggcgcctccaggggcggggagagctgtcggcctgcctgcaccacgtgctctgggcagccgagtgcaggggtgtccagcagaggagctcggctgcctgaggccctgccaggggtgccggcagccagccgggctcagctgagccctgagggggcgcttcagagcactctcagcttgggccgccaccgtgggcagcagaagcacccagtcctcacttcccctggcatggccccagaggcccctccctgacatggccttggccccagaacccagtggggacagactcgcacatacacagggtgccgcctcctgctgtccccagccctgcctctgacccccctgtgaccgcctccttccctggcccaggaggcctggttaccttcatgggggagcatggccccatcccacccagctctgctgtggcccacctttggtcaagcctcagttgtcacatctgtttgggggctcactctgggtgacctaggccacaaggcccacggggcatcaaagaggcagtagcatcttctcccctccccagagggcagagccccccaagcctacttcagagctcccttctgacaccggtagcccgcagccggtattccagaatgggttctggtttaggcgtgaggcctcccccacctcctccacctgcttggggcatgaacccctcccccacgtttccaagcgagtccccaaggtgggcagatgaagatgccaaggatgtcgaccagtctggatgggtctggggtgggggggcatgcggcagacagggaggcattctctggctggtgctcctcagaggagagaggcctccggagactccagacagccttttatggagctgaaagtggcttcagagaaatgcaaagtttcctggagagaacgtggggcgtggttcttgcacagcctccctacagggtggctccagcagtggagctcccctcccaggacccctgggtgctagtgggaggcagtgggcaggtgcagattctcgtccttcccactactgcacaccctttgtctgcgaaggcgcccccagcggtgggtgaaggaggagggacacttggggacccagctgtgcacgtgctctcagtgactgtggagtccactccagggtgggtcccgagggaggggcaggagaccaggggacccacccctgcaaagtgctccgggtcctgacccgtggccaccccatggaacgtaactgagcagccagtgccttgttcctgctggacatctgtggagacaagagtgacttacggctgcttaaagtcagaaacaggttgaaggaggtggaggcgtgggaaagagtctaggaaggtgtttttgccctccacgtggcaaaggttacatttaaaggtgatgctgggtgttctccctgcactaggcattcctggccccaggtccccagcaggtgtgcacatgctgcatacactcacgcatgggggtttcagggcaggtgcgcccttggctccgtgggaggccaggtgaggaacgtccagtgccaaggagcttccgggacagctgtcacttccctttacaaccaggcagcggatagggtcaaatcctggagctttggtgtctaattctgggtggctcctaatctaagcacagacagcaccacacactggggtgggggcacgagcttctgaaacaacgtggccccagtgactccacgctgtgtgtgcccctggagacgggggggtgcacaaggtgcggagccagctagaacctgtcgctccctgcagaagcggtttctgtgtgcggttctgatttgcctcaatgagaaggttttcattcatggctcccggctctcagactgggtggaactgctcccatttaaaggggaaaagaggtggctcggctcgttaaggatttctttttctaagttgttacggcgcccagcagccggctttgtctccccttcagggtggctgcctttcttcccggcccctcgccggcggccctctctttaacaaggccgaagttgtttattctctcgggatgaagtctcggatgggccgccacacccctggcggcccgtgggggcccctctccctttgtgcctgggtcggctcccattcagctcccccgaccccccttgttcccgggcgctcagtggcgcgagatgaggcgatggggccgacaaagatgccacactcatccctgccgacgtccggctcccagcccagggcccctggttcctgtgcagaattcctcgtgggtgtgacaaaaggctgcccccaggctccgctggggtgggggccaggccaagaggcacatcccacactggcccacctgtccacggtaggcgcatgactgccctgaggaggggaggccggcattccccgccacaaaccaggacgtaattggtggcagggctctctgtggaaagagccagtctgctgtttgtctaggaggtcagtcacagaggccccgagacgcccactactgcagcctggcaggcggatgagcccagtatctggcagtgaccagagggagttttgtgcagaccacaaaggctgatgggccgccctagattggtgtccctcttggaagtgggcccagatgtgcgggacagtccccaggaagccccaggtgagggcactggtgccctcttgggaaagctgctccctcctggggcccggctcccggcccagtcctccaggggtgtcccatggtgactggtgctaggaaccccacacctcttcccttacttgggaagtcactggaattgttgggctacatcagacggcccagaaaagtgtttttgtcatcggccagaaataggagagttgtgagtagagggcccgggtggagttggggtgtacttggtctgtgctctgaaggtcactgtgacagtcatggtcccatggtaaggggcatgggttgctggaagagctcttccttcccgagtgagccaagccgggctctcctggcgccagggcctgagccgcagccacaccacagccgccctgaaggctgccggccagggcttacccctcaagggacacggaatggcttcatcagtaccctgcagccccgtggcctggcccgggtggaggcctaggcttcagccatgcgatgtcccttcagaatatgacttgtctgcaatccctgctgctggggggtggcaggtacttggggtgagggttagggtcatagaagcgacatctctacgtcctcatatttgcgtcatctaattttgtttttgtgaatacgtgataacattcacaaggctcaagatgctaaaaggatgagaaggcagtgatgtccccatcacctgtcctgtgtcttcccgtggctttctctttccttggttatgtttgagtcaacagtggggctgacgttccaggagggtccgtgggccaggctcttgctctccgagtgcccagggatggctggaggctgaggagggcctggatgtggagcctcagataccgagtgcttcccttcaggccgggccgcttgctcagagccagcacacagggatgcccggatcacgggggccctgagagggtcccctgctcacagcctccttccctctctccttctgcctcagatgtgaagttcatttccaatccgccctccatggtggcagcggggagcgtggtggccgcagtgcaaggcctgaacctgaggagccccaacaacttcctgtcctactaccgcctcacacgcttcctctccagagtgatcaagtgtgacccggtaagtgagggtgatgtcccaggcagccttgccggggcttacagggggagacacctagtgccacggaaatgccgaggctggtgccaaggcccccaagggtgacaaggttggggctggggctgggcccctcggaccccaggccacagactgacagggcaccggcttcttccactgctcctagaacttactgactggctgggaggtcctcacagccttctcacgtcccctggggcttccaggagccgtagagtttctgggcgaagcgtccgggacggaggccccaggcggccccagccaatggtctgtgtggtgatggtgtgtggggttaggcccaggcgagctttgtttgggccacaatgtgcgtggccaataaatagatgcttgaaaagggctcctgtgaggtccgagacaccggacaacgggcggatagagacagccttgttgtttacggcctctttgagaggctgctgctgttaaaccctgggatgactgtgtctttcttcttaaaaatgccattgttttattcccgagtcttttcttaaagaaagaattaaaatgacaatcaaaagggtttgtggcatttaccaaattagaccagagaggtggccgggtcagccgccggccccgcggtgtgtgagggagtgaccgcctgaccccagcttggggctgggtgggcctgcaagacccgttttggctctggcctgggccgcctcttggtggtctgccctcgagcctcccggggactccgcacgggtctcagcagatgctatctagggtccacctgcctgtcccctgcctagtggtgcctctgtcccggggacactgggagtagcggctgcccagcccatgtgtgtctcggaagaggaagaagcttttttgccgtgggacaccgaagttggcaggggcctcccttctgtgttctcggccatggcctcccttgcaccctgccccgtgttatcctttgggggtggtgaggtgtcctcacccgctgtagggtggaggccagcagcccgcagctctctcaggaaaatggctcagaaacaccatcgaggcctccagaagcccagcaaagagaaagcccctccatcaaaatgaaactcgcgtctgcacttttcatttcgaactccacgccctgagtgaaaaccgcttccccgccaggggtgactgccctgggatgttgctgtcttcgggcagttgtgggaagttgggcgctggcccttatttgagtagagaccatcttaactagattggaggcacacgtctcacagctgacagacacacggggtgaagttacccgaggcggagtccactctgcctgatcagctagtgaccaacgtagctgagcccagactcagaaaaaccgtccacagcagaggcccctgcattttctagggcgtgttctagaattttctttggtgggtggaatgtccatctgtgcaaatcgggtgcgcagtgccacacaccagtgacttttcgcggaggagcgtgctgcctttttggagcttctggctgtgggagaacagctttgtccaccggggtagccttgcaggcagctgtggggccagaggaatgaaggaaggtcctggagtctagctgcatgtgtgaccctggagtgggtcatgggcgagggacgggccgcaggtgaagaatccctggatggagctgccaggcccctggggctgagaattgaagctggctggtgttttaggttgaacgtcaggagtcttgtatctcaccccaggcctctggcctcagtttccccatctgtacagtgggactgtttgtgcagccagcccggccagcttcatttgccatgatgagaatttatctgaggggcgggagaggaaagccctccctataaaggtacaggcgctaaaatgtcgtgacctcagtggtccacctaaaagtcgttctggcctgggtcatcgcctgtcgtgctatgcctttgtccagccccttctggttgggagttaagtggcacctgtgcggcacgtggtggggctgtggcccagccctgctccttgtggaaggtctgtttcctgggctgcctagagacttggcttgaagccctagcgtggcttcctggcagttgggacacacacagccccaacacatggagccggttctccatccagaagcccccgggcagtaagcagccacttcaggctgcgtgggacttgcccgtggtggagcctaggagaggcccctggctgggcgtggcgttccagatttcacggctgctctttcccactgacagtgtggtgtggacgctgccaagggagtctggagccccagagggtggaggtgcaggacttccaggagcgtccgtcgcactccacccgagggcgagcacctcagtggccgcagtgggtggatgcatgctgtgccaggctgatggctggccccggggcacaggcctgagcgggagaggatggaggggagggatcaatggtccaggtccccctggccacccagcattcatcctcagtcatgcacggcccaaggcttcgacagccattgatcatggaaggccaggttcacctcaagggctgccacatggagaggttaagtctgaaaaggctgaaaaggcagggttcaaagggcctcctgtccagatcagatggcactgaattccccagggagctggcacggccagtgggaacaggcggtgaaggcgctgttggacatggggacgggcagggggtgtgcagggtgggcgggcaagcatctggtgtcttgtggctccagagaccaggtgggaggtggaggcatttggtcctgagtgtcctgacaggtgatggcagctcccacatctcgctcaggttcagaggaggcagcatgggccgagggacagtttttggcttagtcttgctcttataaaggcttccgggtcatggcacctgggaaggggccctcgctgcaggccccttctaaggaccccctcttcccacctctccccaccctctctctctcaggactgcctccgggcctgccaggagcagatcgaagccctgctggagtcaagcctgcgccaggcccagcagaacatggaccccaaggccgccgaggaggaggaagaggaggaggaggaggtggacctggcttgcacacccaccgacgtgcgggacgtggacatctgagggcgccaggcaggcgggcgccaccgccacccgcagcgagggcggagccggccccaggtgctcccctgacagtccctcctctccggagcattttgataccagaagggaaagcttcattctccttgttgttggttgttttttcctttgctctttcccccttccatctctgacttaagcaaaagaaaaagattacccaaaaactgtctttaaaagagagagagagaaaaaaaaaatagtatttgcataaccctgagcggtgggggaggagggttgtgctacagatgatagaggattttataccccaataatcaactcgtttttatattaatgtacttgtttctctgttgtaagaataggcattaacacaaaggaggcgtctcgggagaggattaggttccatcctttacgtgtttaaaaaaaagcataaaaacattttaaaaacatagaaaaattcagcaaaccatttttaaagtagaagagggttttaggtagaaaaacatattcttgtgcttttcctgataaagcacagctgtagtggggttctaggcatctctgtactttgcttgctcatatgcatgtagtcactttataagtcattgtatgttattatattccgtaggtagatgtgtaacctcttcaccttattcatggctgaagtcacctcttggttacagtagcgtagcgtgcccgtgtgcatgtcctttgcgcctgtgaccaccaccccaacaaaccatccagtgacaaaccatccagtggaggtttgtcgggcaccagccagcgtagcagggtcgggaaaggccacctgtcccactcctacgatacgctactataaagagaagacgaaatagtgacataatatattctatttttatactcttcctatttttgtagtgacctgtttatgagatgctggttttctacccaacggccctgcagccagctcacgtccaggttcaacccacagctacttggtttgtgttcttcttcatattctaaaaccattccatttccaagcactttcagtccaataggtgtaggaaatagcgctgtttttgttgtgtgtgcagggagggcagttttctaatggaatggtttgggaatatccatgtacttgtttgcaagcaggactttgaggcaagtgtgggccactgtggtggcagtggaggtggggtgtttgggaggctgcgtgccagtcaagaagaaaaaggtttgcattctcacattgccaggatgataagttcctttccttttctttaaagaagttgaagtttaggaatcctttggtgccaactggtgtttgaaagtagggacctcagaggtttacctagagaacaggtggtttttaagggttatcttagatgtttcacaccggaaggtttttaaacactaaaatatataatttatagttaaggctaaaaagtatatttattgcagaggatgttcataaggccagtatgatttataaatgcaatctccccttgatttaaacacacagatacacacacacacacacacacacacaaaccttctgcctttgatgttacagatttaatacagtttatttttaaagatagatccttttataggtgagaaaaaaacaatctggaagaaaaaaaccacacaaagacattgattcagcctgtttggcgtttcccagagtcatctgattggacaggcatgggtgcaaggaaaattagggtactcaacctaagttcggttccgatgaattcttatcccctgccccttcctttaaaaaacttagtgacaaaatagacaatttgcacatcttggctatgtaattcttgtaatttttatttaggaagtgttgaagggaggtggcaagagtgtggaggctgacgtgtgagggaggacaggcgggaggaggtgtgaggaggaggctcccgaggggaaggggcggtgcccacaccggggacaggccgcagctccattttcttattgcgctgctaccgttgacttccaggcacggtttggaaatattcacatcgcttctgtgtatctctttcacattgtttgctgctattggaggatcagttttttgttttacaatgtcatatactgccatgtactagttttagttttctcttagaacattgtattacagatgccttttttgtagttttttttttttttatgtgatcaattttgacttaatgtgattactgctctattccaaaaaggttgctgtttcacaatacctcatgcttcacttagccatggtggacccagcgggcaggttctgcctgctttggcgggcagacacgcgggcgcgatcccacacaggctggcgggggccggccccgaggccgcgtgcgtgagaaccgcgccggtgtccccagagaccaggctgtgtccctcttctcttccctgcgcctgtgatgctgggcacttcatctgatcgggggcgtagcatcatagtagtttttacagctgtgttattctttgcgtgtagctatggaagttgcataattattattattattattataacaagtgtgtcttacgtgccaccacggcgttgtacctgtaggactctcattcgggatgattggaatagcttctggaatttgttcaagttttgggtatgtttaatctgttatgtactagtgttctgtttgttattgttttgttaattacaccataatgctaatttaaagagactccaaatctcaatgaagccagctcacagtgctgtgtgccccggtcacctagcaagctgccgaaccaaaagaatttgcaccccgctgcgggcccacgtggttggggccctgccctggcagggtcatcctgtgctcggaggccatctcgggcacaggcccaccccgccccacccctccagaacacggctcacgcttacctcaaccatcctggctgcggcgtctgtctgaaccacgcgggggccttgagggacgctttgtctgtcgtgatggggcaagggcacaagtcctggatgttgtgtgtatcgagaggccaaaggctggtggcaagtgcacggggcacagcggagtctgtcctgtgacgcgcaagtctgagggtctgggcggcgggcggctgggtctgtgcatttctggttgcaccgcggcgcttcccagcaccaacatgtaaccggcatgtttccagcagaagacaaaaagacaaacatgaaagtctagaaataaaactggtaaaaccccagcgtggtgcctgcctctttgcttcctgggctggccgtgagccagggacgcgtgtcctggtgccctagaaccagggcagggtggcaggcttggcggatgtgggaggccgcagcctgtcctgtgcgctgtgggaagttcagcagcatcctgacctccatccccgggatgacagtcacgccacccgccgtgacaaccaagaatgtctcctgacactgccacatccccgggggtggggacagaatccagccaggagcaggcacacccctcccaactgggaggaagccctcagcacaggtgtgtgaggtgggaggcggtgtcctgtccccgggaggctccagagaataatttgcaggctgcctggctgggtgagcccacctccaaccacgcgagacaacagctccggcctgggtgacgtgagcggtgcccattgatggggaacatcttccccctcttccttgccccaccagtttgtcttcccgggttatttgcagataggaaaataaataaagccggcattcgttaaccctcttctggcgcaaactgctgtttgctctggatgaatcatggtcctttggcgacgccaggctccgggagagcaaagcaccgtgtcagggccatgatccggggtggcctttcactgggatcgtggggacctggaggccgccttataggacacccatgacgcccacctctggatttcaggtgcacgtgactggacttaacttcaaaccccagggtggaggcaggtagtgggagtgccctgggaaggtgtcctcggaccttggtcactgctcctgaacccatctgtgaggctggtttgtcctcatcccaagctaagtggaagctcaggtcccaagccaccgatgggtgctacttgtcagctgcaggttgaatctccgtggcctttatgaagcacctgctgtctacccttcctgccttgtagagcactcctcccagggctcaacagtggggccggggtggtcggtgtgttggctccacaggcgcctgccctgggaggaaggtggggtgtggagggaaacgcttggcccctgtaggtctccaccagcctctcccctgagggtgggggctccgggagccttcctcgagggagtcctatattgagtgggtgggggagcctgcaaggtgcccctgacaggtcacatcagaaagagctcaagggacagtcggagccagaggtgacactggtggccactcgggtggctcacaaggcccagctcctccttgctcctgggcaaattactctgaaggcagggaccaggtctgcaccattgcggctctccagttccaggcaatggccaggtcctgtgtcagggctggggtcctagggaagccatgtccccacccccggcctgcagctgggtttacattcatcccccgagagcacatgggtgtagcaggaggcctgtgcagagagctccgaccatcgcacagggcacctttggttgtttcacggagcaggcaagggagccatcggatcctgttaggtttgagcaaggatgtggggaagaagctggagagccactttgccatgcagggagaggagcacatgggtctagggatctactttagtgtttggaaggttttttaagatgaaagagggatgtgtaggctgataggtctggcagagccaaaaggcagcgacatgtctactgggagagatggagctgagcgcggggctcaggcagggtggcagggcagggccggggccctgggtgggtcaggtgggttcacagccaagtgtgtagagagggcttgggcccagagtgaagcagttgcaagctctcccacaacccattctctctgtctcggcatctgtggcatcccgtgatgggtgggtctgtacacaccccacccctggctgtgccacaatgggggtgtctgtacacccctcacccctggctgtgccacgatgggggggtctgtacaccccccatccctggctgtgccacgatggggggttctgtacatcccccagtgatg

SEQ ID NO: 2 - RNA, 4304 nt, Homo sapiens
cacacggacuacaggggaguuuuguugaaguugcaaaguccuggagccuccagagggcugucggcgcaguagcagcgagcagcagaguccgcacgcuccggcgaggggcagaagagcgcgagggagcgcggggcagcagaagcgagagccgagcgcggacccagccaggacccacagcccuccccagcugcccaggaagagccccagccauggaacaccagcuccugugcugcgaaguggaaaccauccgccgcgcguaccccgaugccaaccuccucaacgaccgggugcugcgggccaugcugaaggcggaggagaccugcgcgcccucgguguccuacuucaaaugugugcagaaggagguccugccguccaugcggaagaucgucgccaccuggaugcuggaggucugcgaggaacagaagugcgaggaggaggucuucccgcuggccaugaacuaccuggaccgcuuccugucgcuggagcccgugaaaaagagccgccugcagcugcugggggccacuugcauguucguggccucuaagaugaaggagaccaucccccugacggccgagaagcugugcaucuacaccgacaacuccauccggcccgaggagcugcugcaaauggagcugcuccuggugaacaagcucaaguggaaccuggccgcaaugaccccgcacgauuucauugaacacuuccucuccaaaaugccagaggcggaggagaacaaacagaucauccgcaaacacgcgcagaccuucguugcccucugugccacagaugugaaguucauuuccaauccgcccuccaugguggcagcggggagcgugguggccgcagugcaaggccugaaccugaggagccccaacaacuuccuguccuacuaccgccucacacgcuuccucuccagagugaucaagugugacccggacugccuccgggccugccaggagcagaucgaagcccugcuggagucaagccugcgccaggcccagcagaacauggaccccaaggccgccgaggaggaggaagaggaggaggaggagguggaccuggcuugcacacccaccgacgugcgggacguggacaucugagggcgccaggcaggcgggcgccaccgccacccgcagcgagggcggagccggccccaggugcuccccugacagucccuccucuccggagcauuuugauaccagaagggaaagcuucauucuccuuguuguugguuguuuuuuccuuugcucuuucccccuuccaucucugacuuaagcaaaagaaaaagauuacccaaaaacugucuuuaaaagagagagagagaaaaaaaaaauaguauuugcauaacccugagcggugggggaggaggguugugcuacagaugauagaggauuuuauaccccaauaaucaacucguuuuuauauuaauguacuuguuucucuguuguaagaauaggcauuaacacaaaggaggcgucucgggagaggauuagguuccauccuuuacguguuuaaaaaaaagcauaaaaacauuuuaaaaacauagaaaaauucagcaaaccauuuuuaaaguagaagaggguuuuagguagaaaaacauauucuugugcuuuuccugauaaagcacagcuguagugggguucuaggcaucucuguacuuugcuugcucauaugcauguagucacuuuauaagucauuguauguuauuauauuccguagguagauguguaaccucuucaccuuauucauggcugaagucaccucuugguuacaguagcguagcgugcccgugugcauguccuuugcgccugugaccaccaccccaacaaaccauccagugacaaaccauccaguggagguuugucgggcaccagccagcguagcagggucgggaaaggccaccugucccacuccuacgauacgcuacuauaaagagaagacgaaauagugacauaauauauucuauuuuuauacucuuccuauuuuuguagugaccuguuuaugagaugcugguuuucuacccaacggcccugcagccagcucacguccagguucaacccacagcuacuugguuuguguucuucuucauauucuaaaaccauuccauuuccaagcacuuucaguccaauagguguaggaaauagcgcuguuuuuguugugugugcagggagggcaguuuucuaauggaaugguuugggaauauccauguacuuguuugcaagcaggacuuugaggcaagugugggccacugugguggcaguggaggugggguguuugggaggcugcgugccagucaagaagaaaaagguuugcauucucacauugccaggaugauaaguuccuuuccuuuucuuuaaagaaguugaaguuuaggaauccuuuggugccaacugguguuugaaaguagggaccucagagguuuaccuagagaacaggugguuuuuaaggguuaucuuagauguuucacaccggaagguuuuuaaacacuaaaauauauaauuuauaguuaaggcuaaaaaguauauuuauugcagaggauguucauaaggccaguaugauuuauaaaugcaaucuccccuugauuuaaacacacagauacacacacacacacacacacacacaaaccuucugccuuugauguuacagauuuaauacaguuuauuuuuaaagauagauccuuuuauaggugagaaaaaaacaaucuggaagaaaaaaaccacacaaagacauugauucagccuguuuggcguuucccagagucaucugauuggacaggcaugggugcaaggaaaauuaggguacucaaccuaaguucgguuccgaugaauucuuauccccugccccuuccuuuaaaaaacuuagugacaaaauagacaauuugcacaucuuggcuauguaauucuuguaauuuuuauuuaggaaguguugaagggagguggcaagaguguggaggcugacgugugagggaggacaggcgggaggaggugugaggaggaggcucccgaggggaaggggcggugcccacaccggggacaggccgcagcuccauuuucuuauugcgcugcuaccguugacuuccaggcacgguuuggaaauauucacaucgcuucuguguaucucuuucacauuguuugcugcuauuggaggaucaguuuuuuguuuuacaaugucauauacugccauguacuaguuuuaguuuucucuuagaacauuguauuacagaugccuuuuuuguaguuuuuuuuuuuuuuaugugaucaauuuugacuuaaugugauuacugcucuauuccaaaaagguugcuguuucacaauaccucaugcuucacuuagccaugguggacccagcgggcagguucugccugcuuuggcgggcagacacgcgggcgcgaucccacacaggcuggcgggggccggccccgaggccgcgugcgugagaaccgcgccgguguccccagagaccaggcugugucccucuucucuucccugcgccugugaugcugggcacuucaucugaucgggggcguagcaucauaguaguuuuuacagcuguguuauucuuugcguguagcuauggaaguugcauaauuauuauuauuauuauuauaacaagugugucuuacgugccaccacggcguuguaccuguaggacucucauucgggaugauuggaauagcuucuggaauuuguucaaguuuuggguauguuuaaucuguuauguacuaguguucuguuuguuauuguuuuguuaauuacaccauaaugcuaauuuaaagagacuccaaaucucaaugaagccagcucacagugcugugugccccggucaccuagcaagcugccgaaccaaaagaauuugcaccccgcugcgggcccacgugguuggggcccugcccuggcagggucauccugugcucggaggccaucucgggcacaggcccaccccgccccaccccuccagaacacggcucacgcuuaccucaaccauccuggcugcggcgucugucugaaccacgcgggggccuugagggacgcuuugucugucgugauggggcaagggcacaaguccuggauguuguguguaucgagaggccaaaggcugguggcaagugcacggggcacagcggagucuguccugugacgcgcaagucugagggucugggcggcgggcggcugggucugugcauuucugguugcaccgcggcgcuucccagcaccaacauguaaccggcauguuuccagcagaagacaaaaagacaaacaugaaagucuagaaauaaaacugguaaaaccccaaaaaaaaaaaaaaaa

SEQ ID NO: 3 - DNA, 63 bp, Homo sapiens
gatgagattaaactgcgtcttcttcgtggtttgaacgcaagagctccctgaacaccctggcgc

SEQ ID NO: 4 - DNA, 633 bp, Homo sapiens
tctagaatgtcaaagagttggactcatacggtgtgtagccttttcagacgggcttctctcacctactaatatcgtggaagtttcctccatatcttttcaggccttgatagctcgtgtctttttagcgctgaataatattgcactgtctggatgcaccgcggctcaacccttcacctactgaaggacttgtgggttgcttccaagttttggtcattatgaataaggctgctgtacacatcggtgtgcaggtttttgcgtggacgtctcaactcctttggataaaggcgaggagcataattgctgcactgcatattcggttagactgtgattagctttctaaaaagtggttttgttagatgtaaaaaatgaatatgacattctgaaacagaaaaaaataacttactctttatctgagtgggatgagattaaactgcgtcttcttcgtggtttgaacgcaagagctccctgaacacctggcgctgccattggcgtgaacgaggggaagcccctcctgacagctggatggtaggacaaagccctctaagccccctctccccgtcacatccccccgaccctgcccacaagggaacctggggcactgggtgttcacctgcctcccactaggtgagatct

SEQ ID NO: 5 - DNA, 176 bp, Homo sapiens
gagaggctgtgggagcggaaggaaaatcagagaaagaaaagagagcactttgttgggttcggggtgttgtttcacccacgggcccgcggacacccatggggccagggaaccctggtcaccgtctcctcaggtgagtcctcacaacctctctcctgctttaactctgaagggttttg

SEQ ID NO: 6 - DNA, 168 bp, Homo sapiens
tttttacagtacctgccccaaattaagaaaactgttcatttacttccttttgaatatagttcagtgtcatcaaaccaccgcccctaattttgactactggggccagggaaccctggtcaccgtctcctcaggtgagtcctcacaacctctctcctgctttaactctga

SEQ ID NO: 7 - DNA, 151 bp, Homo sapiens
caatatgggctgaattcctgctgcccctccatttggctgggaggagactggaaactttgctgtgagagggcatattgtagtagtaccagctgctataccacctttactactactactacggtatggacgtctggggccaagggaccctggt

SEQ ID NO: 8 - DNA, 166 bp, Homo sapiens
ccgcttttccaccagggcggctggcgggcgggcgcacccaggtggggtcacctgcagccgcatacctggcggcgacttgaggtctcgactactggggccagggaaccctggtcaccgtctcctcaggtgagtcctcacaacctctctcctgctttaactctgaagg

SEQ ID NO: 9 - DNA, 951 bp, Homo sapiens
cggctcaacccttcacctactgaaggacttgtgggttgcttccaagttttggtcattatgaataaggctgctgtacacatcggtgtgcaggtttttgcgtggacgtctcaactcctttggataaaggcgaggagcataattgctgcactgcatattcggttagactgtgattagctttctaaaaagtggttttgttagatgctaaaaaatgaatatgacattctgaaacagaaaaaaataacttactctttatctgagtgggatgagattaaactgcgtcttcttcgtggtttgaacgcaagagctccctgaacacctggcgctgccattggcgtgaacgaggggaagcccctcctgacagctggatggtaggacaaagcctctaagcccctctcccgtcacatccccccgaccctgccacaagggaacctggggcactgggtgttcacctgcctcccactaggtgagatctttcctttttggctcctctatcctaatcctcacccacagcgttcccacggtggcctttgcagctaatatccagcgtccagagagtccctgggcttggttagcgttcctcccaaggctgaccctgagctaaagatgtagggacaggggaggtggtcccaggaagctcaggtagacagagagaaaagcccaaagtgtgtcttttgatgagcaggtaacctcaatgggaacccagggtccattccactggagacccttggtcctccagaaggtggagaggctgggacatggggttctccccgacggcttcctctcctggctgctgagggtctcctctcaggtgctgaatagaacaactctaggggtctgggctggagaaaccctaggcagaggagcagagaaggcagctgccccaggcaggtgcagacaggcagagggatgcatcactggggccagggaaccctggtcaccgtctcctcaggt

SEQ ID NO: 10 - DNA, 375 bp, Homo sapiens
tacttaataatatacaatgggctgggcgtagtggctcatgccctgtaatcccagcaaaaagggaggctgaggcaggtggatctcctgaggtcaggagttcaagaccagcctgaccaacatggtgaaaccctatctctactaaaaatacaaaaaataaaagaaatagctgggcatggtggcgagtgtctgtaatcccatctactcaggaggctgaggcaggagagtcactagaacctgggaagtggaggttgcagtgagccaagattgcatcattgcactccggcctgggcaacaagaatgaaaggtctaaatcacttatactactactacggtatggacgtctggggccaagggaccacggtcaccgtctcctca

SEQ ID NO: 11 - DNA, 448 bp, Homo sapiens
gcggcaggtggatcacttgagtcaggagttctagaccagcctggccaacatggagaaaccccatttctactaaaaatacaaaaattagccaggtgtggtggcatgcgcctgtaatcccagctattcgggaggctgaggcaggagaacagcttgagcccaggaggcagaggttgcactgagccaagatcgtgccactgaacgccagcctgggtgacagaacaagagtctatctcataaaaagcaaaacaaaacaaaaaatgatactagcaataactgccgaatgcatttagcctttgctgtacccaggcactgggcaccctgcctttctcactgagtttacttagtgcagacaggctttaagtaaggtgggaaactaaaccccccatagcagagtcgcctgggctggttcgacccctggggccagggaaccctggtcaccgtctcctca

SEQ ID NO: 12 - DNA, 493 bp, Homo sapiens
ttcgtaacctaaacggtaaaaatgttattaatataaacaaatatcgagagggagacctataacatgagcgatctggtaaggactcctttataagggggtctctcttttccgagggtagattctgggacaacgaggcagccttatacagggtgtacgtcctgagcacatcttcagaggtggtcactgcttttaggactgatcagcaggaggtatgcagctctgtgcagagaaggttatgcggctgggtgggggagccacttttgatgtgtgataataaagtcacgggaagaatttcctagcatggctaagtatcccatgagcagtgcccaaattgctctgagccaggcttggcatcgggatctctaatcggacttctttgcagagtgagctctcatgtggccttcaaattaatgctgaatcaccccccaacacatctcccttacagtactactttgactactggggccagggaaccctggtcaccgtctcctca

SEQ ID NO: 13 - DNA, 346 bp, Homo sapiens
actactaaccatcactcaacaacacatactaaatatcacctccaccactaatgtcaccatccccaccaccaccataaccatcacccccaccatcatcatcactaaaactatcacccccatctccagggcaaggcagccacaggtgagcagggccggtgggaggcaggacgagcaggggacaggcactagagctcagggcaaggcaaccacaggtgagcagggccggtgggaggcatcactcagctcctagattttggcaggagctgggtagttgctggcagcagacagctgaggctggtgaaagtgcagtgcagcctcctggtgccaggaagggagtgtgagccca

SEQ ID NO: 14 - DNA, 518 bp, Homo sapiens
  modified_base (119)..(119): a, c, g, t, unknown or other
aaccaccattctcttccttccatccatccatccatccatccatccatccatccatccgatccatgcatccatgcatccatccatccatccatccattgctccttccatccttccatctncccttttcttccctccttttttcatttggttcctgacaacccagctctttgctgggctgtgagcactgcaaccctgggctgtatgcagccagcctttggttgagaacacaagcaccctgctccatagcagnaacnccctatggctagttacgtgaaggctccgtnccctgncatgagctgggcctggcctggcagctgttcccgcaggtgagcaggggcaggtggggtgcaggaggagcagggggcaggcactggagctcaggggaccagggcagagcagccgcaggtgagcaggggcaggtgggtggcaggaggagcaagggggcagctcctggagctcaggagacagggcagagcagtcgcaggtgaacagggcaggtgggggcagagtagcaaggc

SEQ ID NO: 15 - DNA, 70 bp, Homo sapiens
ggcttgttggtattcagacctgaggaattggactgtcggaaatgcttttgatatctggggccaagggaca

SEQ ID NO: 16 - DNA, 93 bp, Homo sapiens
gagctccctgaacacctggcgctgccattggtgttggagggaacccgcatctgactactggggccagggaaccctggtcaccgtctcctcagg

SEQ ID NO: 17 - DNA, 89 bp, Homo sapiens
gagctccctgaacacctggcgctgccattggcgtgaactaccagacttgactactggggccagggaaccctggtcaccgtctcctcagg

SEQ ID NO: 18 - DNA, 85 bp, Homo sapiens
gagctccctgaacacctggcgctgccattggcgtgaacatgagtgcactttgggccagggaaccctggtcaccgtctcctcaggt

SEQ ID NO: 19 - DNA, 66 bp, Homo sapiens
tactctttatctgagtgggatgagattaaagccccactactggggccagggaaccctggtcaccgt

SEQ ID NO: 20 - DNA, 37 bp, Homo sapiens
gagctccctgaacacctgcgtcgtctggggcaaaggg

SEQ ID NO: 21 - DNA, 36 bp, Homo sapiens
ttatctgcgtgggatgaggagacctggggccaggga

SEQ ID NO: 22 - DNA, 50 bp, Homo sapiens
gagctccctgaacacctggcgctgccattggctggcactggggccagggc

SEQ ID NO: 23 - DNA, 109 bp, Homo sapiens
gagctccctgaacacctggcgctgccattggcgtgaacggggactggggggggggacgggttatctctggcggtactactactactacatggacgtctggggcaaaggg

SEQ ID NO: 24 - DNA, 339 bp, Homo sapiens
  modified_base (256)..(256): a, c, g, t, unknown or other
aagcttcccaagatggggaccttgcagtgacaaaaccaagcaggaaaatcccaggtgagctggaacagtgttcaccctaacagagctgccacattccacgacacttcctcctcctcctcctccctcaccaccatcacctgtttttccaccatcaagacgagcagggcctgtactgagcagtgaggaagctatggttcctaaaagtggggccctcttaccgacttcctctctcctacctcaaaaataatttcttggnggccgggagtggtggctcacactgtaatcccagcactttgggaggccaaggtgggtggatcacgaggtcaagagatggagacc

SEQ ID NO: 25 - DNA, 280 bp, Homo sapiens
ttggggatgtagagacatcacttatctggcctaggatggttctgtgttatagtctggatggtgggtggtactattgttcccatattacagaataggaaactgagtcacagagtgctcccccccccacccccatacatgctggcttccaaggctggtaagggggctggaactggaacgcaggtcttgggactgacctggcttcattgcaagtctgccagagaaacgcaaaaggacaagcagcattccaagcatctggcatggtcatcggtgcaaacacccc

SEQ ID NO: 26 - DNA, 49663 bp, Homo sapiens
cctcatggagcttacattctgatggtagagagacaagaaaacaaaatagatagtgtattattgaaggtgatgagagctctggagaaaaagtaggaaaagagacagatctgggacaagggcgaaattacagtatcaaagatgatctttttagggaagatctccttttaaaaacactttggaacaaagatttaaatgaggtgccagaggggtagcaagtgcatattccctgaggaagacgcctgcctggcattttcaaggaacagccagtaaccaatgtttatctacgtaagtaaggaagggagaacagtaggatgagagttcagagaagagggtaggggatatcaaataatttaaggccatgtaggatttttgagaagaattttgcttttatgtcaagtggaatgagggccactgatgatctgggagtagagtgactatgatccgacatgaagtatactccattttttaactatgtgaacttgtgccaacgttttaacctctaaatctgtttcgtcatttgtaaaacggtaaaaagtattttacctcataaggttgtcgtgatgattaaataagatgatacgataagtgcaaaagatttagcttgtacttaacatagagtaggcacattttctccccttccctgtctttcacttttctcttctgccccttccacctggcgctaggagggggagactggaataaaccttgcagattacagcccgtgtaagagtagaaaggaaaggatgacagttgatgtaaagccttggttaacagacataatagctgggatttaaattcagctttattggtggtttatgatgtggactagaggaatggaactgaaagtctcggaggaggggcgatcctatcaggtacaggcgctgcttttccagccctcaatcctcaagactctcccaagatacatttctaggtagtttatcaacacagactccgggtatgctagcatgtttaattgccccattgtttaatgtcttaactccacgaactttaactgattaatctgtcttctaattaatgtttgaatgactctcctcaggtctaaactaccaaggccatctctacttaaaaacagttgtcttttgtttgtgatttcaggggccctgggtataagcgaagtccctgtttagagaccttgtgatgggttcaaaatatcaagaaagatagcaaaatatcacaagcctcctgacccgagaagattagcgttgaaagggtctgtcgtgtttgtttgggcctggggctaaattcccagcccaagtgctgaggctgataataatcggggcggcgatcagacagccccggtgtgggaaatcgtccgcccggtctccctaagtccccgaagtcgcctcccacttttggtgactgcttgtttatttacatgcagtcaatgatagtaaatggatgcgcgccagtataggccgaccctgagggtggcggggtgctcttcgcagcttctctgtggagaccggtcagcggggcggcgtggccgctcgcggcgtctccctggtggcatccgcacagcccgccgcggtccggtcccgctccgggtcagaattggcggctgcggggacagccttgcggctaggcagggggcgggccgccgcgtgggtccggcagtccctcctcccgccaaggcgccgcccagacccgctctccagccggcccggctcgccaccctagaccgccccagccaccccttcctccgccggcccggcccccgctcctcccccgccggcccggcccggccccctccttctccccgccggcgctcgctgcctccccctcttccctcttcccacaccgccctcagccgctccctctcgtacgcccgtctgaagaagaatcgagcgcggaacgcatcgatagctctgccctctgcggccgcccggccccgaactcatcggtgtgctcggagctcgattttcctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggtacggagcggaccacccctcctgggcccctgcccgggtcccgaccctctttgccggcgccgggcggggccggcggcgagtgaatgaattaggggtccccggaggggcgggtggggggcgcgggcgcggggtcggggcgggctgggtgagaggggtctgcaggggggaggcgcgcggacgcggcggcgcggggagtgaggaatgggcggtgcggggctgaggagggtgaggctggaggcggtcgccgctggtgctgcttcctggacggggaaccccttccttcctcctccccgagagccgcggctggaggcttctggggagaaactcgggccgggccggctgcccctcggagcggtggggtgcggtggaggttactcccgcggcgccccggcctcccctccccctctccccgctcccgcacctcttgcctccctttccagcactcggctgcctcggtccagccttccctgctgcatttggcatctctaggacgaaggtataaacttctccctcgagcgcaggctggacggatagtggtccttttccgtgtgtaggggatgtgtgagtaagaggggaggtcacgttttggaagagcataggaaagtgcttagagaccactgtttgaggttattgtgtttggaaaaaaatgcatctgcctccgagttcctgaatgctcccctcccccatgtatgggctgtgacattgctgtggccacaaaggaggaggtggaggtagagatggtggaagaacaggtggccaacaccctacacgtagagcctgtgacctacagtgaaaaggaaaaagttaatcccagatggtctgttttgcttggtcaagttaaacccgaagaaaacccgcagagcagaagcaaggctttttccttgctagttgagtgtagacagcaatagcaaaaatagtacttgaagtttaatttacctgttcttgtcctttcccctatttcttatgtattaccctcatcccctcgtctcttttatactaccctcattttgcagatgtgttctacatctcaagagttattacagtactccaaaacagcacttacatgattttttaaacttacagaggaattgtagcaatccaccagctaaccgcctgaaatagacttaaacatgtgcatctcctttttttttttttttttgagacacagtctcgctctgttgcccaggctggagtgcaatggcgcggtatcggctcactgaaacctccgcctcctgggttcaagcaattctcctgcctcagcctcccgagtagctgggactagtaggtgcacgccaccatgcccagctaatttttgtatttttagtagagacagagtttcatcatgttggtcaggatggtctccatctgctctgttgcccaggctggagtgcagtggcgccgtctcggctcactgcaacctctgcctcctgcattcaagcaattctcctgcctcagcctcccgaataactgggattacaggtgtctgctgccatgcccggctaattttttgtatttttagtagagacgggggtttcaccatgttggtcaggctggtctagaactcctgacctcgtgatctgcccgcctcggcctcccacagtggcatgtgcatcttatagctgaagtctaagccttcttaaatcttgagatccatcaaaacagacaggttttctaattgttatacaatgtatatgttatgtttataatagaaatcattttacaaataagttataaatgggaaaggtctatttgtaattatcagctcagaattaaccataaaactggtgtcactgaagtgactgaggtccaaaatgctgactctgcatgttatagactacagatatcaaatatggttgctaacaatagtttactttgagactgtagccatccacagtatatttgcttttaagagatggtagatggtaattcagttttatgaaaaataaaaatgaattttcttccattacaaaattgttggattcgagtccagtccactccttactagcttttctaactctcggtgagggatcccctcccagcccatgatcttcatttggtaagactcctttggaacccagttctctctagtggatttaaatgtgatttggttttaaaaatctcattcaaggaattttttttttttctggaaacaaccaccgcataaacaagtaaaccggaagatacatgtggctctgaattcatatatatacacaaactctaatccaatgtctgtccacagtatttcctaggctagtaaactttttggccttaacgacccctctaccctctttgtttttttgagagagagagtctcactctgtcacccaggccggaatgcagtggcgcgatctcggcccgctactacctccgactctcaggctcaagcgattctcccgcctcagcttcccgagtagccgggattacaggctcccgccaccgggctaattgtatttttagatacgggatttcaccatgttggccaggctggtctcgacctcctgacctcaggtgatccgcccgcctaagcctcccaaagtgctgggattacaggccaccacacccggcctacactcttaaaaattatcgaaggggccgggcacattggctcttatctgtaatcccagcactttgggagactgaggcgggaggatcgcttgaggccaggagttggagaccagcgtactcaacatagtgagaccttgttataaagaaaaaaaaaatccaggattaaaaaaaatctttgatttgtttgggatttattaatatttaccgtattggaaattaaaacaatttttaaaaatgtattcatttaaaaataataagcccattacttggtaacatgaataaaatattttatgaaaaataactattttccaaaacaaaaccaaaacttagaaaagtggtattgtttcacacttcagtaaatctctttaatgatgtggcttaatagaagatatggattcttatatctgcatctgcattcaatctattatgatcacacatctggaaaacttgtgaaagaatgggagttaaaagggtaaaggacatcttaatgttattatgaaaacagttttgacctcttgcacaccagaaaagtcttagtaacctgaggggttcctagaccacattttgagaactgttttaggctatgcaaactggttggggggaggttggggtaggcagagagctagaagatacattttagtgtaattctcctcatctattcctaattgctttggcctacatttgaaataaagcgtggaggcaaacgggataagatacatgtttgtagtggttgttaacttcaccctagacaagcagccaataagtctaggtagagcagagtaaggcggggaactatgccgtgaccgtgtgtgatacaatttttctagcctgtggtgctttttgcggcagggcttaggagtaaggttagtatgttatcatttgggaaaccaaattattattttgggtcttcagtcaattatgatgctgtgtatatttagtgtttatctacaatatatgcacattcattaatttggagctactcatcctataataaatagttgtgcatttactcccatttttttctgcatttctctccttatttataattatgtgttacatgagggaaaggaggtgaaattaaacattcatattatttcaaaaaatttgaaacaactaactaaaaaatatgttttattttctgtatggtgtttgttatacaatctgtcaatattcatgcacctcttgggagacagtgtatgaaaagcaaagagtaacagtcacatggattactgattactgagatatattcacttgcatctttttttttttttgagacggagtggctctgtcgcccaggctggagtgcagtggcgtgatctcggctcactgcaagctccgcctcctgggttcacgccattcttctgcctcagcctcccaagtagctgggactacaggcgcccgccaccacgcccggctaatttttttatatttttagtagagacggggtttcaccgggttagccaggatggtcttgatctcctgacctcgtgatccaccctcctcggcctcccaaagtgctaggattataggcgtgagccaccgtgcccggctcacttgcatctcttaacagctgttttcttactaaaaacagtgtttatctctaatctttttgtttgtttgtttgttttgagatggagtcttactccgtcacccaatctggagtgcagtggcgtgatctgggctcactgcaacctctgcctcccgggttcaagtgattctccttcctcagcctccccagtagctaggactacaggagagcgccaccacgcctgattaatttttgtatttttagtagagagagggtttcaccatattggccaggctggtcttgaactcctggcctcaggtgatccacccgccttggcctctgaaagtgctgggattacaggcatgagccgccgcacccggctttctaatctttatctttttttgtgcagcggtgatacaggattatgtattgtactgaacagttaattcggagttctcttggtttttagctttattttccccagagatttttttttttttttttttttttgagacggagtcttgctctatcgccaggctggagtgcagtggcgccatctcggctcattgcaacctcggactcctattttccccagagatatttcacacattaaaatgtcgtcaaatattgttcttctttgcctcagtgtttaaatttttatttccccatgacacaatccagctttatttgacactcattctctcaactctcatctgattcttactgttaatatttatccaagagaactactgccatgatgctttaaaagtttttctgtagctgttgcatattgacttctaacacttagaggtgggggtccactaggaaaactgtaacaataagagtggagatagctgtcagcaacttttgtgagggtgtgctacagggtgtagagcactgtgaagtctctacatgagtgaagtcatgatatgatcctttgagagcctttagccgccgcagaacagcagtctggctatttagatagaacaacttgattttaagataaaagaactgtctatgtagcatttatgcatttttcttaagcgtcgatggaggagtttgtaaatgaagtacagttcattacgatacacgtctgcagtcaactggaattttcatgattgaattttgtaaggtattttgaaataatttttcatataaaggtgagtttgtattaaaaggtactggtggagtatttgatagtgtattaaccttatgtgtgacatgttctaatatagtcacattttcattatttttattataaggcctgctgaaaatgactgaatataaacttgtggtagttggagctggtggcgtaggcaagagtgccttgacgatacagctaattcagaatcattttgtggacgaatatgatccaacaatagaggtaaatcttgttttaatatgcatattactggtgcaggaccattctttgatacagataaaggtttctctgaccattttcatgagtacttattacaagataattatgctgaaagttaagttatctgaaatgtaccttgggtttcaagttatatgtaaccattaatatgggaactttactttccttgggagtatgtcagggtccatgatgttcactctctgtgcattttgattggaagtgtatttcagagtttcgtgagagggtagaaatttgtatcctatctggacctaaaagacaatctttttattgtaacttttatttttatgggtttcttggtattgtgacatcatatgtaaaggttagatttaattgtactagtgaaatataattgtttgatggttgatttttttaaacttcatcagcagtattttcctatcttcttctcaacattagagaacctacaactaccggataaattttacaaaatgaattatttgcctaaggtgtggtttatataaaggtactattaccaactttacctttgctttgttgtcatttttaaatttactcaaggaaatactaggatttaaaaaaaaattccttgagtaaatttaaattgttatcatgtttttgaggattattttcagatttttttagtttaatgaaaatttaccaaagtaaagaccagcagcagaatgataagtaaagacctgtaagacaccttgaaggtcatggagtagaacttccatcccaagcagatgaggatttatttaatctcaaagacctccaggaggggacattccccaactgtccttgttaactcattttcagaacatatttattagcatattttacatgtaatttggatcttcatgttaaatttaacatcagtggagatggaaaataagcatatcgccttgtctttgaaatagccctatattgttagattgtttcttaggcttctttaccctgggttaagcagtcctaatactttagcatttattctacatctagtgtactaatttaaaaaaatcagttctgaaaaatttctaagaactttcttcaagttccaagctgtgaaatctagaacaggtcaaagtgccttattaacgtactgtactgtgtagtgtcttgaagagacactttgcgctgaggcaagttctgagggcattgggtggccttgggaagatatttatgcagtttagaacctggagaattgattagataactaatcataaggaaacgtcacatatttttggtactataaaaaagtggagaaataatgcctatttgcaaagatttgatttaaacatagaaacaactttatttggcttccaattttaagaatttacagcagtaaaggggaacagtctaattgaagtagactgcctatgcaatagtctctgtatatttacttttgacaagttaattcaatgtgtactatagttttgtttctttgaagaggtttgaatagtgcacccattttaatctgtattgcaaattcagggttacttggcagactctactatttaaatcagatgtaaaaggaagttttaatataattcactttatgcctgaaagttttcctgggattttggaaggtgattttactggaaatgctgtctgtcttccctgaaaatctgagaaattccattacactttgtttccaatcagaggtcatgagtgctatatgagtatatacagcatgacgtcatgaatgtgataaagtgggttaggaaaccttttgctaatgattgttaaaatgcaatataaatgttgaagaaataaagctaacagttaagcctttatttgggcggaaggctgaaaaagtttataaacttaaacctataactctgcttatgatttctgccaaaccagaagacttgactctgggaagcattggttacctgtgaactttgaaactgacggtccctgacgtagtttagtcacctgggaaaaggtatctgagattatctcttatctcccaagttacagtgagtctctgagggaactgacacattacattaagttcttggtgtagttaaactgtaagaaaggcaggagaacttagtagttaaatagttggttaaatggaaatgctgactccatgttattgtaaaaagttaaaaatttaggaggatatggggatttcactgccattgcaggttttgattggtatttaccaatccgtgtgggtcagagagaaaattagaaaggatatgactgcacattttggaattattagcagtttttctacatttaaaatggaaataaattttttaaaaatttaaatcaagtaatactgtattttttggtgatttagatttttcaaaatttacactaagagatagtaaggagggtggctattgtttctttcaataatgtctctgagaggttgtaactcatctaaggatacgtagctaataagtggtaggatttcaatttaaattctctgagaccaagttaagtagaatttgcactgtactcttgtataactttttaaaactgaaaattagctatctttcaaattaagaaaatatttactaatggagactaattcagatttgtaagtataccaaaatttgaacttagcctgctatctaatggcaacttagtggcagaggtatgatgtaaaatcattcaggtatgacacatagatggagtatgtttgtattcgaggctgtgcacataatcacctttacttgtattgtgaagtatatattgttatcttttatgaagcccactaaagagataatgaaatacctcgttattagggcaagattattgaaaactcaaaatagcccccaaacacaatacttggctagaaatatatacctttatagttcagagatcatttattatcaaaaccctgaagttttttttctaaggtaaaatttggtggaagaggaaaagtctcgttttaaaaaaatgtaggtagttacagagatcagaatgattagttgatcacttaccaaatatatattaagtatctactgtatataatatgctagtaagaataaatatagcaggaagtattttttcccaggctctaattgtttgacatcagcatgcttttattgtggcacttataattcagttcaagtattatgcccctctttgatggaacagtttcctattcagtaaggaagaccagattaatcattggattggtttgtttcatctttagtgttctgagctgtagagtatttatttaccaaggtttattttaatttttattttatttttatttttccatgttcattgtagaattcattttacctacgaatgaagtatgtagattatagagagaaaatttgtaaaattaaactgatactgaagactggtataagaaaagccttatgtaatttgtaagctgctattcttctgagtttatacatatatctttagtaatcaatgagggatggttgggtgactgccctccaggggacatttggcaacatctggagatgtttttggttgccacaacttggggagagagtactgctactggcatctattgagtagatgctattactttaaatggcaaagctgcagttacctttgcaccaacctaatattaaacttcctgcagtgcacgggaaagcccccacaacagggttatctgaccccaaacctcaatggtgttaagatccaaaccttgatatgttaacctgtagctttaaacatcctttaaattgtcaaattcatgtccctgacataaggtttatgttagattttcaagtataacaaagatttaaactttaacttttgtacgttaatgatatgttagcttactccagtcttctattaaaacattctgtttttaaaatcagagacacacagcaattttataaatcatttctcttcaaggctgtgaagctctccccacttttgtgagtgccctctactggtcaaattatttgctttataacaagtaacagtgaaatcctaagtttgtgtagtttcgctgtttaaattatgggtggcatcaatttataaatatattcgttttatttaaaagtcttatatgattgatttcgtatcatttttgctctctgctaatattaatataaagattactgtctgtattagttaggcctaactaagtaggtgagtatagtgaactaagaaaggaaacgaggcagtatataagaaaatagggtggttcagttgttaacacttactgagcttactttgttgaagggactaaaaggcagcagtgtggctctctgagcttctttgcatgcactcaggagctgcttaatggagtccaaggcttggtggtgtgttacaggggatgataggagggtcctattcagaagtggcaaattgtgaaagtgcacattttgtagagttttataggactgtagaatagttgtgagcacctgatttttagaataaacagaaaactcaggtactgtatttaggtcaaattaagaataagtatttattaagacctgaatataaaactttactggtcatggtttttttctaccttgggtttttataaatccaaagatttaaaaacatacaaatggaagttggtaatggaattaagtgaaaggaaaaaatgattttatggtttggaatctcctaagattctggttttaacaatacaactaattccttaatcctagaaatgttcttcactgcccactttgtaccatgcagtcttcctgtgggctagagatacactgaggcgcaaaacagaccagattcctgccttcatggagcttattagttttaggtatctctagatttcttgtaatacctattacaatgcctgcacatcagttcattcatgtgggttcaacgtagtactcagtacatggcaaattcaagttttacttttcggaacttcatggatttttttcctcagaatatcttttatccataattggttgaatctgtagatgcagtacccatggatatggatggcccactttattttgaagagcagtgtttctaggcaatcatgctaattatatatgacttaatttagaggctttatacttaagagcattacatttctggcgtctcttaaccattattatttcataatgtgtaggttatggaacagttaaattattgggatcttaatatagaaattagtagaaataagccagatatggtggctcatgcctgtaatcttagcactttgggaggctgaggctattcgctgtactattttttactacttttctataggtttgaaattttttcaaaataaaacattgaaaaaagtaaggtaggtagtgtgtccctccttaatcctttcaaatattttattttcactatttctattaatttttttttttgtttttgagatggagtctcgctctgttgcccaggctggagtgcagtggcgcgatcttggctcactgcagcctccacctcctgggttccagccattctcctgcctcagcctcctgggtagctggtattacaggcatgcaccaccacacccaattactttttgtatttttagtagagacggggtttcaccatgttggccaggctagtctcgaactcctgacctcgtgatctgcccgcctcagcagtgtcactgcttctagaccgttttcaaggcacagagcttagaaatgcatgttactaagaaatcaagagttaactatttttcaccttctttctcccgcagtgagaaccctggttctaccctgtttctccttgtgtaaattttaatgctaaactatacacttgtgaaataaaaatgataatgtcattcttaaattatggatcttgcagtgttatctaagtaacatagattgagtgatttaactttaggtttccttatttgtggaatttggataaatatttttcacccttgagaaaagtgagactcctttctcatcatcagagtatccttaaaccattaaggcaaacatttgggaaaaaactgagctatctggctgcataaaaattaagttttctttaacaaagatagaagacaaatgaaaacctagaaaaaccatttggttcaagtaacaggaagctatcttatatatgaattagagaaaagcaaacacacaaatagaaaaaaagggatggggggtactaaagatataaatagcttgtctaccaaaaaagaaataaaataaataacatgaacatataaaaagacacttacttcatgaatgtgatgcaagttcaaacaataaataacatttctgtactttcatattggctaaggttaaaatgataactgctaggaagggtatggagaagtgtgcgccttgcactgtagtgggagtatagaccctcagactttatggaggtcagtctggaaatatgtttcaaaatgtaaactacatgtcctttgaccaggtaattcaacttcttgaaatttatccaaggatttaattggataaatgtttaagatgtatatataagaatgtttactgcagtgttgtttatgattttaaaaaaatggaaatcatcttcatgtctaccaatagagaatgggtgaataaattatggtatgtccatatatacaaattacatagttgttggaaatattaggtagatttagatatactgatgttcaaaaatgtccattatgtaagtgaagctgggtcacagcaccttgtgttgagtatgatttcatctagaaacaaaattactccctcatcctttgttgtgttttagttttttaaaataagcttataccattgggctgggggaaaagtaaatactcgttttggagagagaaaagggcactaaagtttcagataccgttagattatttcatgcttatttttcaagcctcaataaattacataattcacatgtagtcttggattaaggaaattgctattaaggctaaataaataatatgagaggtatataatataaaatatgaacattatattggcattaagattggatccacggtcattccagcctctcattcttacctggacttcaagtgatcacttgtgggcaaatgccatctgacttgaacaggttacacatgtatgctcattatatcgttattttcaaaatttgtcatataaattttccttgagttcattcagatttttgaactagttttttctcttgggagtagtacacacttaattctctctagtactaagctaatgttcaccattcttataattttaagtatccagcatttagtaaagaagtctttgttttctttatccttacttttagtgaatgtcttagtttttaattgaaaattctgccatgaaaataagctctttaacatcttcactccctaatcaaaacagaaatccttcatagccttcagttgtagctatccttccctgtgatttgtccagctccattatatttattttgaaatatggtgaccagttttgcaaaattatttcaactgtaggtgcccagtgattttgtaaggagaagatactgtttctgaacagttctcagtagccagtggcctgcccctactttttggcctgcgtgtagtatataaaataatgcagttaactttttatagcacttttcattttataaagagattttcatggtctttaatattaatctatgtataaagtcctgtatgcagttttacctactttcacagctgaaggaacaatagcttagagaagatgtgagataaagtagtttgcccaagcccatagcacaaataagtgaagttcttcggctgtccatggatcgaagactcccaagtctatctctagcctggacttctgtcctgagcaccagacatgtatgtatatcaagatgcctgcaggtcatatccaccaggacaacccatgagtacagggaattcaacatgcccaatatcactcatcttttccttcgccctcccctttgtactcatcccctgtcggtaagctctgttattttaaaaaattgaaatgtattcacatagcatacaatttacacttttcaagtgtacatggtttttagtatattcacaagggttgtgcagtcattactactaattccagaatgttattatcaccccaaaagtcccacatccattagcagccactccccaatcccttctcccaccagcctctaaaaactgctaatttttccatctctgtggatttgtccactctgattatttcatataaagagaatcgtacagacgtggccttttgtgtctggcatcctccacacaggatgatattttcagagttcgtctatgtttttgcttgttgatcattccttcattcctttttctggctgaataatactctgttatatggatataccttattttgtttatctgttcatttgatgggcatttgagtgatttcctctttttggcaattttgaataatgccactataaacatttatgtacacgtttttgtgtgaccatatgttttcacttctctcgggtgtatatctaaggtacagttgctgggttatatggtagctctgtctttgactttttgaggaactgccaagtggttttggtagtgattgtactgtttacattcctaccaacaattttacctaagtatttctcaaatctatttaatcttttcggtccatactgctgttgctgccttagttcagattttgtcatttcttgtaataattcgtagctcatctcccagtctctgctcccctctctccctccctcccccttcttctctctcttatttccacccatttttaacatttatagaagtcaaaagtctagttcagaaagcagaaaccacactagatatttcagcacagagaactaattaggtgttggaagactgaaaggcaaaaaaacactgaagtaacacagtaacatcaagaatgggcactactcctaagattcagggaatgctgggaagatttggggtttatcagaactggaagctcagaggaggggccccttgtcgctgaggcttaatccctgcagaggtgcctttggctgctactggtgaatctgagtgggtatggatgagtcagtgtctgggaagggccaaaacattttgtccctttctataatttgtcatgataatgctagtaatgaatctgatctcccttcctattttaaaaaccttttagtgattttgtataggatgaagtttaaaactccttacttaatatacacatgaccctccgtaagctggcccctgcttgattgtccagtttcacttcttggtgcttattctaaggcctctaagccttagagatcctctaagcctttgagatccccaaaccctggactgcggactggtacccacctgtgtggcctgtgaggaactgggctgcacagccggaaggaggtgagcattacttgccttagctcctgtcagatcggcagcattagattctaataggagcgtgaaccgtgttgtgaactgcccatgcaaggatctaggttgcatactccttaggagaatctaactaatgcttgatggtctgaggtgaaacagtttcatcctgaaatcacccccaactcggtccttggaaaaattgtcttccacgaaactggtccctgatgccggaaaagttggggaccgctgttctaagctaaagttatatggagctccttggttctgtgtcctcaacatgctgttctatgttttttacattctgtttgctccttcctgcttggaatgtccttcccctccccgtctttcttaatgcatacaaagttgatctctcctgtgtgccaccattgtacttcgtcttgcatatggtgttacattcattttattttaattatttatttacgttcatgtctcttccactcaccttagttgcttgaggtcagaaactatataatgtgtgacacggaatgtgacacctagattttcaataagtgtttctatgatacaagggagactgatgtgggtagatgggaatgaactcatcaacctctgtttacataccctaaattccctgtttcttccctattataattctgacagtctacaacagtctttgatggcttataaacggaaagtgcggaacacatcattctacagtgaatttaaataacctttcggaagagtaacgtaaagtacttgagcattaattgagtaaaagtttctcatcttttcctacaggtgttattaagcagtatgtaaaaagtccttacaatacttaatacattaagaaaacatacaatttcaagaggaaatccccgagtaatacattattgacattttcagcagttctagttatattgagaagagcatctcatggaattggcagaatgaagatggagattaaatgagatgatgtttgtaatatgcttatgacagtatctggcatataagtaagggctcagtaaatgttgactgctgtaattactattaatagtaatatgattacctttagtaaaagttattagtttctttaggttttttgtttactacaatatagtaaacaaaatctatacttggaatgtatatattgttttgttttgatacatggaatatgtctctgtgtcagagtcactgcctgagttggaaaacccatactcgagtatgttaaaaggtgaacacactgaataatttagttattaattataatggaaaaatgacaaacttgatgttctggttaatgaggttatcttatcttgaatgagttagcttttaaattcctcaaaataaaggcatttaataaaccaggaaacacttcattaaaaaaattatgcaagtcagtgtaaaagaagattaaaattccacatgggcaaaggacacacgttggcgataaatatgcagataagaaaaaaaacctatataacattattactcctcaaagaaattggtatgaaaacaataaaaatgtgtagcttatcaaaccaacaaaaatttaaaaatatgaaatccattttaagtaatgataaaatgggtgcactcttagtgctttatagaatagtagtataatgaacctcatgtgtgtaccaaccagctctttcatatcttaacatttagcaacatttgatttagctctttcttttttccaagatagaaaagttaatattgttgaagactcctgcattcttttccctagtcttattttcttccctcccataaatgtgttaaaatctctgtgtgtattgttttggttgtatttttacataaaactttacatattatataaaatttaattgaaggtaaaatttattaaattattcttaatatatattgtaatttaaaaattaacagcttcattgtcttgataaaatttatggtatcttaaacatgtgcttgtttttctaagagaacattgaaacatagattttaaaacaaattgttgaaagattaaaaaatctgcctttgcacactgttacattgaaagtggggcatttgtcgtgaacattcatttcaaatatgtagtatcttcagaatatttgagaaggatttgtattatataattgaaaaatctgttaaattgtatttatgttaactgcttaattctaataaaatttccattcattttttagtatctgcatatatttacatcaaatggattcattcacttatttaagaggcagtactaattacctatagcgttcaagactgttaggtagagggtgtgtagtggtgagtacaacaggcgtgagccctaccaacacggagtttaaagcctagtagaggatatagacttaaacaatttcacaagtaaatacataattacaaattataatacatgctatgaaggaaacataggaggtaccagagaaggaagagtgctttgcatttttatttttaagaccgaagagtgctattggaggactttgagcaagtgaatgacatgatctaacctaccttcgttcattcattcattcattcattttcttccttcctggctcaagcagtcctcccacctgagctccccaaatagctgggactacaggtacacactaccacacctaatttttttttgtattttttgtatttttgatgggattttaccatgttggccaggctggtcttgaactcttgacctcaggtgatccacctgtctcggcctcccaaggtgttgggattataggtgcctagcccatggtgcctagccctaacctacatttataaactatcacttgctgctgtgtggagactatattgtgagattaacagcagggatacctgctaggaagcaattgctgcagattgcctgagacaaaatagttatcatggactagggggatggtgttggtggtggtggtaggtggttggatgtaggatatattttgaagataggtaaatggtgcaagattatgggtcagttttaaatgcttaagtaaattttctttgtaagacattttaggatgccatgttaagaatctctttataactgtcatttaaaaaaaaaccacatattttcttagcataatttcccatagtaacattactatgtcaaaggctatgaacatttgaatgactttagataaatactgtaattgctttccaaaaatattgtgcttattatgtcaccagaaatgtttgaattctgtctacaattcagtcttgccagtatagtacatttcatttagaaaaattttttactatgtagatggaaaaaataatattttagctgggagtggggggactatggggaataactttccttcatttaatattttattgtgagttagtttaagttactttattttatcgtagtttcctaaggctacaaattagtaaccttggtaacttatgtacctaatttaaaagtttacttttttgaaaggctggaaatactaattaaaaacgtaacaccttcatccttgtctttgctccattattaactagtttcattacagaatctctgtgttttaaaatcagatgggttttcataaccagtactttctcagagtggtaaatttaaaaaaatatataaagagaataaataatatttgttgagaatacttcaaataatgtgaagagttattaacttacagcaggagttggcaaacttttctataaagggccatatgggtctttgtcacaaagtcttgggtttttgtttttgtttttttaaacagctatttaactattcctagctaatgggcaatacaaaaacagtgggcaagatttggcctgtgggcagtagcttgctgaaaccttatttagactctaaattttttgaaagagtctacattgatgcatatttttttttcttcctccaaatacagttgacccttgaacaacatgcgtttgagtgaccatgggtccacttgtgatacacgtttttttcccaaccaaatgcagatatggagggctgacttttcatatacctggatgttcctgggccaactgtaggactagaggctggggggggtcttggaaccaatgccgtgtgtataccagggatgactgtttcttatggcctgacctgaagttggaacagaatctttattaatatataatttttgttgcgtttgttttctctttatatttatccattctttttagatcgtatttcatttaacactttttcttctttagtttttaccaagttgcactgaaaatagctcagtgactaattgcacttctaagagtgaggaccctagttaaaattaactctaaaaatactgaatttttaacctaaaccttttgtttctaatcaacagtattatttatgagtaggttatagattactttgaaacggaatgtgtctcagaactttgctatcgatatttttaaggtctggtagggaaaagataataggaatgagatttatcagtgaataggggactgctttcccagtttctcggtcgcactggtgtattcaccatggaagcatcttatgaaatatgtacataaactactaatatcccacattacaggttgactattctttatctgaaatgcttaggacctagaagtatttttggattttggtttttcagagtagggatactcagcctacattggtaagtaaagaatgtgaggtgacaggctgggcgcgatggttgacgcctgtaatcccagcactttgggaggccgaggcggatcacctgaggtcaggagttgaagaccagcctggccaatctgtactaaaaatacaaaaattagctggacacagtggcacgtgccagtagtcccagctactcaggaggctgaggtaggagaatcgcttgaacctgggaggcggaggttgcagtgactcgagatcgtgtcactgccctccagcctaggcaacagagcaagactccatctcaaaaaaaaaaaaaaaaaaaaaaaaaaagaatgtgaggtggcagcaataggtaggaagagtctttggtcagctttacatgctctgtagccatgcctgggtaatgggttgactctaagactctgtgctttgctcccacctcctgctttttcattactctttagaatggtttttaatttgtgatctataggagttctttcaagtatttaataagagaataggctaaattaagtaaatgtcaactgaatgctcaaatctctactaaagagcctcttatttagaaaataaatatccatcttttttttctgactggtgagataattaatttttattacagatggtttggaaaataccatatgctttaaaagataagcacaaaattatagtctaatatgtaggttttcatactttaaaaaattgaaaaccaaagaaaaacatttaacatagcatctagtacaaagaaaagagataagcaagagataaatgtcttttttgggacagagttttgctgttgttgcccaggctggagtgcaatggcacaatctcagctcaccgtaacctccacctcccgggttcaagtgattctcctgcctcagcctcccgagtagctgggattacagtcatgcaccaccaggcccaggtaattttgtatgtttagtagagatggggtttctccgtgttggtcaggctgatctcaaactcccgacctcaggtgatctgcccaccttggcctcccaaagtgctgggattacagacatgagccatcgcacccggccaagataaatgtcttttaaattatctccattaaagacataacctttataacattttgatgtatatattaccagtttttaaacacatagtagatttgtataaatacataaacacatattattgtgatcatgctgcacttagacatctttatattctccttatactgtaaacattttgaaatactttactaacaacatttgtaatgaccattctttctctctttctccctctgatagaatggtctacagagtaattcataaactaaacatactttagaggctgggcgcagtggctcatgcctgtaatcccagcactttgagaggctgaggcgtgcagatcacgaggtcaggagttagagaccagcctgactaacatggtgaaaccccatctctactaaaaaaacagtacaaaaattagccgggcgtggtggcgtgcacctagaatcccagctactcaagaggctgaggcaggagaatcactcgagcccaggaggcagaggttgtagtgagccgagattgcaccacagcactccagcctgggcgacagagcgagactccatctcaaaaaaaaaaaaaaaagatacattaatactatagcctacatgtggaacattaagaaaataattgcttttatgtttatgctttatacctgttgttagccctgcttcttatttcatgatttcatggcttcacattgtaacatccctttaccatattttttgaggactgttttggcagaatgtgtgaaatcttgagcagaagtattacccaaaagtcagaagaaaatcagatttttatttcaagattctgttaaagttacccactcccttcttttacttaatcttatagttgcagttctctctctttttagaaaagaaaaaagaggcccctcaggatttgcagatgaaacaatattgctctttagagatatccatctggctgttagattatttttccacagttttcagaagtggatgaggccattagaatcttgagtattgcccatttccttatgtgtgcctttgactatagataaaatagatgcatgacaattatttataagttgattgatttttcttgtcatttaaatcatcttgaataatagagttggtagagctatcccatttttgaaattattttgttttgtcaataactttttgttaccagcatgtacacttgcattgttgactctccatataatacctttaaaaaatttttttttgtggtaaaatatgcataacataaagtttaccatggtagttttctttcatttgttttgtttttgtttttttgagacggagccttgctctgttgccaggctggagtgcagtggagcgatcttggctcactgcaacctccgcctcccgggttcaagcaattcccctgcctcagcctcctgagtagctgggactacaggcgcccgccaccacgcccggctaatattttgtattttaatagagatggggtttcaccatgttggccaggatgttcttgatctcctgacctcatgatccgcccacctcggcctcccaaagtgttgggattgcaagtgtgagccaccgcgcctagaccatggtagttaattttaagtgttcaattcagtgaccttaagtgtgttcataatgttgtgcaaccatcaccatgttgtctaaccattagcactatctgttttgagaacttttttttatcatcccaaattagaattctgtacctgtcaaatagtccccagtaatcctccctcccccagcccctggtaatctgtagtctacttttcgtctttttgaatttgcctattttaggttcctcatataagtggaattatgtggtatttgtccttttgtgttggcttacttcatttagcataatgttttcaaggttcatctgtgttgtagcatgtatatacaggttgaagcatccgttatccaaaatggttgtgaccagaagtggtttggatttcagattttttttttggattttggaatattcatagatacttaactggttcagcatccctcgtccaaaaatccaaaatcagatggagctcagtggctcatgcttgtaatcccaacacgttgggtggccaaggcaggaggatcgcttgagcccaggagttcaaccagcctgagcaacacaagaccctatctctccaaaaaaaaaaaaaaaaaaaaaaagatgaaagaaaaaaaaatccaaaatcaaatgctccagtgagcatttccttttagcatcatgtcaggctctaaaagttacaggttttggagcattttggatttcagatttttggattaacctgcattaatgctcaacctatatgaaattttattcctttttatggctgaataatgttccactgtatgtatatactacattttgtttatccattcatctgttaacagacacttaagttatttccacattttgggtattataaatagtgctgctgcgaacattggtgtacatgtatctgtttgagtccctgtttttagttattttggttatatacctaggaatggaattgctgatcatatggtaattctgtgtttaactttttgaggaactaccactgttttccacaatggcatcaccattttacattcccaccagcaatgcacaaagatttcagtgtctgtatccttgctaacacttattttccattttttgagtttttttgttttgtttttttaataatagccaatcctaatgggtatgtggtagcatctcatggttttgattttattttcctgactattgatgatgttgagcatcttttcaggtgcttagtggccatttgtccgtcatctttggagcaggaacaatgtcttttcaagtcctttgcccatttttaaattgaattttttgttgttgagttgtatataacaccttttttgaagtaaaaggtgcactgtaataatccagactgtgtttctcccttctcaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattataggtgggtttaaattgaatataataagctgacattaaggagtaattatagtttttattttttgagtctttgctaatgccatgcatataatatttaataaaaatttttaaataatgtttatgaggtaggtaatatccctgttttataaatgaagttcttgggggattagagcagtggagtaacttgctccagactgcatcggtagtggtggtgctgggattgaaacctaggcctgtttgactccacagccttctgtactcttgactattctacaaaagcaagactttaaactttttagatacatcattaaaaaagaaaaccataaaaaagaatatgaaaagatgatttgagatggtgtcactttaacagtcttaaaagcaatcgtgtgtatagcatagaattgcttggattggataaacagtggcattatatattttaaaaaataaaagttttgaaagattgaagaatttgggcattacagttctcttaaatctgacaaagctgcataaaactattaaaataatcattattatactattttatattctatttctttgagggtttagttttccaaaaactacatattaagcaaatgaatcactcagtggctatgtcatataataacgagttagcctagttataagaagtttaacattttatttaagaacattgttacagcatgtttactgtatagtctagtaatagaggaaaagacatttgggtgggtggtagtggtagtatttttatagaggagttaccaaatttcagctctattatccaagtttacccagctaatggtgttcggaaccgggaatttgagccaattctgactctgttgtctgctctgctccttcttttgtgctgtgtctttgaaagtcacctaaaattgtgagggaatgtaatttcaccccaaatttagagtttatgcacttgttatattgaaaatgattaacatgtagaagggcttttaatggaataagtggtgtagtaacttcagtgttgcctacctagaaatcaaaatctttctagttgtccactttgttttttgaaaaagtaatatgaaaattatgttaatgctttaattcaggtttttgtaaaatattttttatctttacacatttaacatacgtttctaaaattatagtctgttatatagcactttgggtctagaatttttcagtagtttctgttttactattatgatctacctgcatattaacctattaggttatagttttactatacttctaggtatttgatcttttgagagagatacaaggtttctgtttaaaaaggtaaagaaacaaaataactagtagaagaaggaaggaaaatttggtgtagtggaaactaggaattacattgttttctttcagccaaattttatgacaaaagttgtggacaggttttgaaagatatttgtgttactaatgactgtgctataacttttttttctttcccagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacaggtaagtaacactgaaataaatacagatctgttttctgcaaaatcataactgttatgtcatttaatatatcagtttttctctcaattatgctatactaggaaataaaacaatatttagtaaatgtttttgtctcttgagagggcattgcttcttaatccagtgtccatggtactgcttttggctttggtttctttctacattgaaaatttctcttcaattctgagcacatgttaacatttagaattcaagaggtggggatttttttttcccatggttacatatatatatatatatatatatatatatatatatatatatatatatataaagaacagggcaacaaatttttgcgttttctatttcggtagtacttttaaaccattatgtcatgtttctaggttaaacgttgttgtatttgaagaattttactttggcagaatttttttgaggatgtgtttatttctggagaaaggtctcattaaagaaagacaatacccagaaagccaacagaaattctgttactcatttaatgcatttttctgacaaaaattattgccagagagaacctgaattttgtttcaaaaatcatctttgttttaaaaatgactttttcttcaggtaaaataaaataatttcagttgctattatttaacctgtttgtatgaagagtttaacatataggaaatgaatacataaagataggaaggaattaattgttatatgtagtcatatgtctcttaatgacagggatactttctaagaaatacattgttaggtgattttgtcattgtgcaaacatcatagaatatacttacacaaaccttggtagtataacctactatacacctgggatatgtagtatagtctcttgccccagggatacaaacctgtacagtatgtaactgtactaatgactataaggcaattgttaacacaatggtaagttttgtgtgtctaaacctacacttgggctaccctaagtttatatatttttttaaatttctgttcaataataaattaaccttactttactgtaactttttaaactttttaatttttcctaacattttgacttttgtaatacagcttaaaacacacattatacagctatacaaatttttctttccttatatctttattctgtaagcttttttccatatttaaaattttttgtttgtttttacttattaaacttttttgttaaaaactaagacatgcatgcacattaacctaggcctacacagggtcaggaccatcaatatcattgtcttccacttccacatcttgtcccactggaagatcttcaggggcagtaacacacgtggagctgtcatctcctataataacattgccttcttttggaatacctcctgaaggacctatccaaggctgtttatagttaacttttttttttttttttttttttttttagtaaataggaggagtacactataaaataacaatataggtgctataccattatacaactgacagtgcagtaggtttgtttacaccagcatcaccacaaacacgtgagcaatgtgtcgtactacagtgttaggatggctataacatcactaagcaataggaacttttaaactccattataatcttatgggaccactatcacatatgcaatctcctgtggaccaaaatgtcattatgtggtacatgactgtactaagaaattgatccatctatattccatcaatttgtttagggctttttctggttacatttacctgtgagcccagaaaaccagttttgtagaaattaacttctgtaatgctaggagttaaaaaaaattgctgaacaacttttacattgttaaacatttaaaaacaagcgttctagaagtttatcaaatttcataaaggtgcaaaaatgtaaatgtaaatcattatccagctaatatatatgttgtatttccctagtaggagagcatatgtacctcttcctagttatacaaatttgatatatagtaaagaaacagtaaattctacttcaagtcattttgggaggattaaaaactgaatttctctagtttgaccattgtacagatttatctggcaattttactaaaacctgatttataggttaaacttggtgtatatcatatatcactttactttagaggaattaagatttcacataaatccatttccaggttccaaagaccaggaagaggcttggtttttgtttttctttttactgtctttacagtctccttgacttttcttaggagagaaggtactgagaaaacatgattctaatatttattattttttcttccaacattttcttatgaaacattttcaaatacaaaattgagttttatttaaaacatttgcaaatatactacctagattctaccattgttgttttatatttgctttacttacaacttttaaaagatgctttttataccactgaacattttagcttacatttcacaaagaaaagaaaaaatttaagagactttgcataatgttttaaggggttgcagtaaagaagtgcttcttatattttcttatgcatacaaatcagctgggcttattaaaatccagattctaattcagaaggtttaggtggggaccgagtctgcatttctaacaaactcctaggtggtatttttcttggtacttggaccatactttgagtagaaaagcagtagaggacataaaaagagtcttgttagtcccactttgttgctgtccacttctcatttgataatatcctaaaatagctgtgtctcctttttggtggttgtatgattactacctcagaagtactaattgattcttgctatttgaccttaatactttaatataacacagcattcatatttgatcagaaaactatctggcttccttttataagagatttttaggttttatacagttttgtggccttgggtttttttgtttgatttgtttttttgaaggtatataatatgtaagtagataaacaaatttgatttgtagacatttttatgtggatcatctaattaaaaatggagggatacagtatgaaagaatacttgtacttcttaacagagcactcaacctttcttttacatcctgtttcactgatgttattatgtaatttatgttgctaaactataaattagatatttaatttctgttctttgatttccttttattattaaatggacttgttgatttgcctagaaattaatttgcctttcaaaagtcttattaatcttcctccgttgaaattaatttgatatttgcatgcttctggaagactttaaagagctattccgagtaactgtagagattataaaatgaaatatgggaattttaataaattttacatctccagttactggtgaaaatgtcaagtcctcctttctgcagagtattttgttactcatctgttattcagcttatttatttatttatttatttatttatttttctttctttcttgttttttttttttgagacggagtcttgctttgtcgcccaggctggagtacagtggtgggatcttggctcactgcaggctccgcctcccgggttcacaccattcttctgcctcagcctcccaagtagctgggactacaggcacccgccaccatgccttgctaaatttttgtatttttagtagagacgggtttcactgtgttagccaggatggtctcgatctcttgacctcgtgatccacctgcctcggcctcccaaagtgctgggattacaggcatgagccaccgcgcctggcccttatttgttttttaaacaaaattagtgtgcatatccttgttgtattttatcggcaagttgttttatgccctaacttttggggtcttgatcatgagcctaaaacacgtaaacacccaaaaagaattatattccggttaaaggaacaaaacattcatttagaagttctcatccatgtaaatcagaggctggcaaatattttctgtaaagggccaagatagtaaatgttttaggctttgagggccacaagtggtatctgttgcatttttttttaattatgaccctttaaaatgcaaaaatcgttgttagcttgtgcatagtataaaaataggctggccgcatgctgtggctcatgcctgtaatcccagaaatgaggtgggaagccgaggtgggcacaccacctgaggtcaggagttcgaggccagcctggccaacgtggttgaaaccccgtctctactaaaaatacaaaacttagccaggcgtggtggcgggtgcctgttatcctggctactcaaggggctgaggcagtagaattgcttgaacctgagaggcagaggctgtagtgagcccagatcaagccagtgcacaccagcctggacgaccgagcgagactctgtctcaaaaaaaaaaaaaaaaggctgtggctgcatttggtccattggctgtaatatgctgattcctaattctctgggtaactttagtgtttgattagctactagaagttaggttaaacttttgtattttacaggctaactttaataatcttaaagtaaaacttaacatagttcatggaaaggaaatagaaattttaccctagtactcttttttttttttttttttttttttgaggcagagtctccctctgtcacccaggctggagtgcagtggtgggatcttggctgattgcaacctcctcctcctgggttcaagcaattcttgtgcctcagcctcccgagcagctgggactacaggcacgcaccaccacacctgactgatttttgtatttttagtagagacagggtttcgccatgttggccaggctggtcttgaactcctggcatcaagtgatcctcccatctgagcctcccagtgtgctgggattacagacgtgagtcactgtgcctggtctctagtattttttttttttttgagacggtctcactgttgccaggctggagtgcagtggcgcgatcctggctcactgcaacctccgcttcccggattcaagcgattttcctgcctcagcctcctgagtagctgggactatgggtgcacaccaccacgcccagctaatttttgtatttttagtagagacggggtttcaccatgttggccaatatggtctcaatctcttgacctcgtgatctgcccgtctcggcctcccaaagtgctgggattacaggcgtgagccactgtgcccagctgtactttttaagataagaattgcagggtatatatttttaccaacttaataacttataattttaaaaagctaattacttggctagaatataatgcgttacatattctttacactcagttcagtccatatctgaaaggcaaatagaattattttctgctagtacattgtgtagtccctatgttcctagtgtataaggactgttacctagttcacatttatctgggttgttgacagattttcctggtccctttggacagtgcatggccatgttggcaaaagctgtcaaaattgaaacattgacaccatgagaattgtgtgttttccagtctgctaaaatcaaaagtgggagggttcagtaaggtgaataacagaagcagagttttcggggtatctgttactcctcattcggcttttctgctctctgggggtctcaatttaaatataatgtgaaaattagttttacgaacctaaaaatgttgagtgattcatttcctggttttgttgttaatttctagatatttaaattaattgttagaagaaccccgttaaagaatgctttgcaaaacaacctccttatgtgctatgtctctgtttaatagtagttgagtttgtgtacatgagatcaatattttgaactatagctttttatgagttaaaaattgacggaacagttactgtgcacttgctgtgcaccatggtagtctcccaagtagtggtttttctgcatttcaatagtacatgagataggctgtgggtggcaaggtttcttgagaaagtgagggatgcacagttgggttttagaatacatcttgttcctccatgcccttccccaccaaaaggctggtagtcttgcatttgtatatagttagggtatttgatgtgttgcttccttgacagagttttgcaagaatttgcagatttaacaggaacaaaaacttacttaaaacaaaatctcttagtaaaagcatagtctagcaagatttagaatgatactttggctaacagtactttctctatatggagtgctttgtttccatagcctcacaagtatgttttcagataatagttgagttgaaaatgttgtcaatctcttgattttaaaaaatttacatatttaaagttgtatacttttgttcctacgtattttcagttgttcttaaagtttaataagtgacatttgaaaatgagtatatgtgtataaaaacaaaagtaggctaggcacggtggctcatgcctataatcctagcactttgggaggctgaggcaggcggatcacaaggtcaggagtttgagaccagcctgggcaatatggtgaaacccccctctactaaaaatacaaaaattagctgggtgtggtggtgcatgcctgtagtcccagctactcaggaggctgaggcaggagaatcgcttgaacccggaggtggcggttgcagtgagccgagattgcaccactgcagtccagcctgggcggcagagcgagactccatctcaaaaaaaaaaaacaaaaaaagaaaaagttaaaaaaaaacaaaaaacccccacaaaatgagtatatgtggcaacaagtcctattctcaaaaaaattattgtgtgctagttaagagcttaatgagtagccagtcggtattaaatatctgtttcagctatattttatctttaaaaattatctacagattttggaatgtgaaaaactagtgttttgtttcataggtatatactgtaggcattttaaaaataagagccagtgccagtggtttacagtgtacacaaggataatgttctcatgttctcttgatgtcagtatgactttaaagcatattatcaagaaataactaagtctgaaaaactgtggtaaataactggtactctaaaacctaagtttcttattactaaaaataagaaatggtaaaagtcaccctgtgctgttaattatatgagccactgaggtcctgacactgaattcttggtggtggataataatctcttctttttaattattggcttccaattctctctgcattgctggaaacaaaaatcatatatttcactattggtggtggggatgctgtcactgaaaaagtagacacattcatattgattttagtagacacattcatattgattttagaaaataagttaaaatcaaaatttgcttctgctaaattagtagaggaccaatactgtttttctccttcatagtatgttttggtacttctacattgacattataacttttttttttttaaacagaaatagaagtttacattcttagaaaatttatgaaaatatgagcttttacctggtttgtgtgtgtgcgtatatatatacacatatttttaaatttcttacattgattttcaaattgaaagagaaccatttgtgaaagtatcttaacagagctcatgctttacattttacatgctacaaagttattttagtgccttaaattatttatgttgcttattaatgaaaattttggatacataattttttcaagacaaaggtaaaaataataaaccctttccttctgaggattaatgataaatataaactttaaaacgattaaaaaaatttttttagagacagggtcttgctctgttgcccagactgaagtgcagtggtgcagtcatagctcaatgaagcctcaaactcctgggcccaggcaaccctcctgcctcagccttttgagtagctgggacttcaggctcatgccaacatgcctaatttatcttatttttagtagagatgaggtctcaaactcctggcatctcttgccctctcaaagtgctggtactacaggcattagtcaccacacctgacacttaaaatcttttatatacaggtgtaagtgggtatctaacttaaagtgccaacgaatgtagttgaaagtttgtagttggcttagctaactagttaactaaattgattccattaaaaataagataagactgctcttagaatataatgatttttgttattcgttaaatataaatatatcactggatagtatatgttaatgacttgagatacgcattttaacatataatcacgttacttaaatgcctgcctttgaactgaaacttaacattatgaatttaaattaaagtttgactttagaggtaaatttctgtactttactaaagcagttcttaatataattctgagatttctaaaaattagtgtgccctaaagaattgaggtgtgtttttcttaactactgtaggcagtagatgtacagatgacttctgcatgcaaaaattaagccctagccattggtttacttcaactaatacttagttgccaattctctgtgtgtgattgaatttaaaactgcaaatggtactggtgatacattaactttttaggtgctaggtccactttgttacatttggttcagtagaaacattgatgttaccaatctcagaaagctaaaatatgtatgccaatccccaaattaggtaatttattcttaattttaagataaaagaatagaattcccttaaaattaaatgtggagtaaaatataccagctttaaaaaatattcacctttctgttagaagaatgaacataatattacatcttttaatttgcactatatatagattaatatttctgtgtatttctctgtgcccctactttgatggtatgcttttctgaacaaactagcagcacagttaactaagcactttgccccgtttgatgactgcctaattttctagattggaaaatattaaaaacttttatctccatatggccaatatatgattgtacctgttgtcatagctctcttatgtttaagcaagaaaaaccctattaagagtatttaaattagaatggaaggcacacagccagtatgattgaacactgttctaaaaattatttttaagacttgtagtaaggccaggtttggtggctcatggctgtaatcccagcccttaggaggccaaggtgggcggatcacttgtgctcaggagtttgagaccagcccgggcaacatggcaaaaccctgtctctacgaaaaatacaaaaatcagtcaggtgtggtggtgcttgcctgtagtcccagctatttgagaggctgaggcagggggatcacctagcctgggaggtcgaggctgcagtcatgatcgtgccattgcactccatcctgggcaacccagtgagaccctgtctctaaaacaaaaaaataaaaaaagaacttgtagtaaggatacaaaatgctcctattttgtgtgtgtcctttaattcatgatgtttttatattatggtaagcagctctcatttaagattttaataatgtaattaaacatgtacagaagacccagtctcagcttcacttgtataccctggaaatagactgaaaggtgttaaaatttaagataaaactcaaggttccagtttcttgactcacctttgagattcttttatgtttttgttgttttttaacaaaggtttcacgtccatattttaccatttttcttctcattctcccctggaggagggtgtgggaatcgatagtatataaatcacttttttcctaagtcaaagaagtaatttaaagctaacttcagtttaggctttaattccaggactagcaaactaaaatggttgcattaattgacaaacagatgctaatacctgtgtttaggcttgtcataatctctcctaattcctaatttaaaaattttaaaatttaattccattagaaaacaaaactgacttttaagaacaaaccaggattctagcccatattttaaaactgcatcctcagttttattcaaacagtctgatgtctgtttaaaaaaaaaaaaatctcaagctcataatctcaaacttcttgcacatggctttcccagtaaattactcttaccaatgcaacagactttaaagaagttgtgttttacaatgcagagagtggaggatgctttttatacattggtgagagagatccgacaatacagattgaaaaaaatcagcaaagaagaaaagactcctggctgtgtgaaaattaaaaaatgcattataatgtaatctggtaagtttaagttcagcacattaattttggcagaaagcagatgtcttttaaaggtaacaaggtggcaaccactttagaactacttaggtgtagtattctaacttgaagtattaaaagataagaaacttgtttccataattagtacatttatttttaatctagtgggaattaattataattgagacaattttgatggctgtagtagactaatctatatttggcataaagtctaatgatttaatgagtcttaagtaaactaaatatttggaaactgatatttacctttatttttaagggaaaagttttgagataatcagcagcttttttttttttttttttttttttagtagggagaaaaagatatgagctatagtagacagcagtaatattgaatggcccagaaggtgggaaaaagccactcttaaatgtattttttcttttggatattttacaagcaaataataacttctgcctaagttcgccatctcagtggcatcagcagcacagcactttcttatcccagtgagaaacctgggaattttaggatgactcctaccgccctcttttccccctggtttggaagtatccacaaattcctgtgacgttacattctgtgtcttttatgtcatcattagttcaggcccctatcatttcttgttggactgttagaacctcctatttggtttaccagttgctgccatcattcattgtgaaaccggagagatacactttaaagaaatgtcatttttggccgggcgcggtggctcacgcctgtaatcccagcactttgggaggcctaggcgggtgatcacctgaggtcaggagttcaagaccagcctggctaacatggtgaaaccctatttctactaaaaatacaaaaaattagccgggcgtggtggcacgtgcctgtaatcccagctacttgggaggctgaggcaggagaattgcttgaacctgggaggcagaggttgcagtgagctgagaatgcaccattgcactccagcttgagcaacaagagcgaaactctgtctcaaaaaaaaaaaaaaaaagtcattttagctatagaataaaatctcatgttccacatgtgttgcagatagtccttactaccttcccaccactccagctcttttttggtcttatatctaaaaacgtcatcttgcctgaatttcttttgttcttctataaataaataccatgttatttcctaccttcccttgagtcttggctcttgtttggaatgccagtatttttatccctagtcttactaattagctaacactctcatgattccccagtctcctactctctaaaaacctttctttaaacccttagactaggcatggagcccttcctgtgtattcccagaatactattcttaactattatatgcttcccatgttatgttgaaataactaacctcttctgtttcattcctatattacttgacagcaaaatcttagccagaattacatatttttaatctttgcacacccattgcctagtaaggttcctgggacatagtaactacccagtaaatatttattgcgtggaattctcattttcgtttctaaacccgtattaaactctgtcttgctcagaaaatacttcactaggtatcataaagttcatggcagagcttaagctttggatgcatattgtttgtaatatatcatgttcttaagaataggcaataaaattacagttttcaaaaactactacatttattatatttattacaagttggtgttctttattacatgaattttaggtatttcccaaaagtataaaatatacatttgaatagtagactcaatcccaaaagatactacgtggtgtactaatctactaaactcagaaacaaagcatgactggcattaatttttgttgaaatttatgaactctgaatgtttttgaatatcattctgtaaagcaatattttgcaattaaagcaattttgcatgttaaattttaccacaacctctaaaatattgcaaatttaacaatacagtttgaaaagttacacattttaaataacagtaccatgaccagatttaggtggtggttttaattttttattttctcctcctattgtctcaccattagatgattttaaaaatagaattgtttagagtaaaataagtgttatgctctaatttatatttaaaatgaaggtttaagcacgtactattctaaaatttctaatttgtgcaaattatgttttatacagtgactgtaggtgaatgtcacaattgtttgatgtgacgaatccttgtttttcagtacacgtggaagtaattcatataaaagagaagtatacttggtaattaaaaatttaaaattaaatacaatttaaaaaaaaatttatttgacaagctggctgtggtgtgtgtgcctgtagtatcagctgcttgggagcctgaggcaggaggattgcctgaccccaggagtttgaggttgaagggagctatgatggtgccatggcactgtagcctaggcaacagaaagagactccatctcttaaaaaaagtaaaaataaaaaaattttggcacagggacagtggctcacacttataatgccagaactttaggagtccacagcgcgaggactgcttgaggccaggagtttaagaccagactgggcaacgtaatgagaccccacctttaggaaataaatacataaataaaaatttgacaatgataaacatatataaattagcttttcttagtcctgaaaaagataatgttatgtgtatgtgtgagaatgattagttctcatatgagaaaaaaagaattcattgctctgtgtaggttgtgacatttccttcacgattgaaattaattaatttttttttattacttatttatttttaaaatagagacaggttcttgctgtgttgcccaggctggtctcaaactcctggcctcaagcagttctcctgcctcagcctcccaaattgctgtgactgtaggtgtgagccactgcactgggccaaaattacttaattttaacaagatgatgtagagaggagagttcattgcaacataagcctagaatctttgtcagaatcttaggaagtaatgttttcaaattctgtgttttcaccataaaatgtgtcttctctgtgtccatcacatggtttttcattgttttctgctttaccattttagtaccattggcatttttcttcattgtaaaagtagtagaaatggagtagattacataaggatgtgatcagagggaatttattcattcagggtaagggagttagatcctcttttaagattctatcacattctaagggtttatgattctaaactgtcaagtaaattgtcaagtgctggcaagctacagaataatttttattgtatcattggaaattttcccctctatatgtgttaaagagtttagcctgaagggatacatacacatacatatatgtaatcaaaccttgatggtattgtattgctgataaattatttcttaccacttttcctttctcctgtgggagaaacaaaagcatatgtttgtgtagtatcagtaatgatattagagagtgggaaacatcagtgagtgcagtttggggactttattggagactttcactagtgctcaaataaataatgctggtttttatcctactgtttgcttaatgtggactagcctcttattcccattctatgtttacctctcttaaaatattggtcacgctttcttgaattatagatctattaggaaaattcatgaactgtagctaattttcattgttcatgctccagatttattttgaaatatcgttaatcttagtagtacagtaaaggagaaataccacttaacattttttgtttttttttctttgagacagagtcatgctctgtcacccagtctggagtgcagtggtgctatctcggctcactgcaatgcacttcgcctctccgggttcagcaattctcctgcctcagcctcctgagtagctgggattacaggcacctgctaccacacccagctaatttttgtatttttagtagagacagggtttcaccatgttggccaggctggtctgaaactcctcacctcaagtgatccacccgtcttggcctcccaaagtgctgggattacaggcttgagccaccgcaccccgcccacttaacattttaaattaatttcaagataatatcacttgaatatttttacacatataatttttttaatacatttatttacacagtttataatatcctacaaagtgattacaatgagtaaaaacccagttttcattgttcctaaagtggcttgatttatacaacttaatgtgttgggtatttgtttctaagactccctctgctgtctaggtttggaagtattgtgaggttaacagattttctttttatagttactactcagttgaacaggctttaaaatacagagagaatcatattttttcttcattttttgcttttatttatatttttcttttaattggagacatgacaagaattgacttgtgtatggatcttgcataatttaagtactgcaggtttaaaatctactctactaccagtttgagagtgccatttttcacactgtagattattaggttgaaaagtattatggcttaaaatcgcttttagccattaaatttaaataaccttgctttaatcataaatagatggtggtcacaatgactaactgttaaactctttgaagacaggatatttggctttatatggcaagcttttgaatacaacagaaattaaaactttatgggatagaaagaatctcctccaaattggtaaactataagacctttcaaatgatttagctaatttctccacaaatctgaggtattagtgttttttttaaagtggtattctcctgtgttggggtcactttaaacctttttcttaatgataaatatatgaattgaaactaatcccttaatatatatcatttgaaaactgaaataatatgtttagatactgtttacttgttgataaattattggaataggatgttcgaatactgtttacttcttggtaaatttttaaatccaatggattttacgtaagtatagaactggagctcaaatactgttactgtgtgtgaagatatatgaacatagtttacagttgcatggcttatatctaaagtccagaaacataaggacaattaagtgtacacacacacacatgcatttggattttgatgacttaggtttgccaatgtggaaaaaatagtagcaaattaagttctcctgtgaaaaagtcgttaccttatttaaaattctgtgccattggttatccttgtcttttgtgaaaattagtgttcctgtttataatattgacaaaacacctatgcggatgacatttaagaattctaaaagtcctaatatatgtaatatatattcagttgcctgaagagaaacataaagaatcctttcttaatattttttccattaatgaaatttgttacctgtacacatgaagccatcgtatatattcacattttaatactttttatgtatttcagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggtttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtgatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccgcacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgactatgagtgtgtatttattcatgaaatttgaactgtttgccccgaaatggatatggaatactttataagccatagacactatagtataccagtgaatcttttatgcagcttgttagaagtatcctttatttctaaaaggtgctgtggatattatgtaaaggcgtgtttgcttaaacttaaaaccatatttagaagtagatgcaaaacaaatctgcctttatgacaaaaaaatataggataacattatttatttatttccttttatcaaagaaggtaattgatacacaacaggtgacttggttttaggcccaaaggtagcagcagcaacattaataatggaaataattgaatagttagttatgtatgttaatgccagtcaccagcaggctatttcaaggtcagaagtaatgactccatacatattatttatttctataactacatttaaatcattaccaggaactgtttgttttgtagtgaaccttgagtatgtgctgttaatataccaaattgggtgaaaaaataagggattcctttcaaaagttaagagaagtaagtgtgtaagaaattattttgcttattaaatgttcggtaaatggcattctcttgtcagtaaaatggagaaataagctaaaaataattggctaagtcctattaagttagaggattaagtgtattatattttcattcaaaattgggtgctcattaatttatgatcggtagtatagctaaattgctatgtttgtatcaaaattgagcataaagttgctgatactttctccgtatgaacagaagttgaaacctatttagttcagtagggcagctcagggatttttttacacaacatgtatatcttcccattttaagttagaattattttacaacatctggtatacataaacagctggcactgatagctaaattaaagtagtaatgatcaattagttttgttggtatctgaataatagcgttgtttcatagctctgtatttcctaaggaagtacaaagcttctagctctttcattacaaattcgccctgtgcaataagttctttgatcttctctggattcttcacatctttgtttttaaggaaaatgttcttcaaacgctttttaaaatagtctgctccttttggatagtctcgtccaagatacagcagcttcaaaaagaaagattatatatttctaaacaatccatgtcatataataacatttttataaaattggcaacataattacttacatttttataaagttttagtacttctcctcttaaagaattggccattttcatttatcatgtaaattatccacttttatgcataacatacctaaagaaaggaaaatttttttgcaattagctgcattgtagtcttaaaaaaataaaaaaaggttatacacattgagaaaatggtaaccttttttacattcaataaatatttcttgataactttttcgttccacgtactgggatatagttataaacacttccgataaaattacctgctgtcataattgacgttttcctatgggagacataagcaaagacaattgtgattgtgagaagtcacatgaaggaaatgagaaagtggattgtcatcacagataggtacgtgtacctccttttatgccacagtggaatgagttaaactagatttaaattccagttgcataatgtacagattaattaaccttgctgagcctgagttttccttatcaacaaacaagagattatctttaccctgctctcaaggcaaggccagagccacttgaaggacattgagcagaagcctgatcaaatgctgatgggtgcttatccaaagggaggctgaaaactagcagaaactgggtgagttaagcaggttggaatagtagatgggcagtaagattggtggtgaagaggccaaatgaacaacctgtaagagggtgtccctgaggaacaggcaaaatcatgcttctttatgtgtaatgtgttaactctactttgtagaggaggctccaaac

SEQ ID NO: 27 - DNA, 20 bp, Artificial Sequence
  Description of Artificial Sequence Synthetic probe
atgactgaatataaacttgt

SEQ ID NO: 28 - DNA, 23 bp, Artificial Sequence
  Description of Artificial Sequence Synthetic probe
tggtagttggagctggtggcgta

SEQ ID NO: 29 - DNA, 21 bp, Artificial Sequence
  Description of Artificial Sequence Synthetic primer
gcctgctgaaaatgactgaat

SEQ ID NO: 30 - DNA, 21 bp, Artificial Sequence
  Description of Artificial Sequence Synthetic primer
ggtcctgcaccagtaatatgc

SEQ ID NO: 31 - DNA, 19 bp, Artificial Sequence
  Description of Artificial Sequence Synthetic primer
acctgaggagacggtgacc

SEQ ID NO: 32 - DNA, 19 bp, Artificial Sequence
  Description of Artificial Sequence Synthetic primer
tggrtccgmcaggcycngg

SEQ ID NO: 33 - DNA, 24 bp, Artificial Sequence
  Description of Artificial Sequence Synthetic primer
tgtcgacacggcystgtattactg

SEQ ID NO: 34 - DNA, 19 bp, Artificial Sequence
  Description of Artificial Sequence Synthetic primer
acacggccgtgtattactg

SEQ ID NO: 35 - DNA, 24 bp, Artificial Sequence
  Description of Artificial Sequence Synthetic primer
gtgaccagggtnccttggccccag

SEQ ID NO: 36 - DNA, 21 bp, Artificial Sequence
  Description of Artificial Sequence Synthetic primer
cagggttgtgttggaatcagg

SEQ ID NO: 37 - DNA, 24 bp, Artificial Sequence
  Description of Artificial Sequence Synthetic primer
tgttccactgccaaagagtttctt

SEQ ID NO: 38 - DNA, 22 bp, Artificial Sequence
  Description of Artificial Sequence Synthetic primer
tggataaaggcgaggagcataa

SEQ ID NO: 39 - DNA, 32 bp, Artificial Sequence
  Description of Artificial Sequence Synthetic probe
actgcatattcggttagactgtgattagcttt

SEQ ID NO: 40 - DNA, 19 bp, Artificial Sequence
  Description of Artificial Sequence Synthetic primer
acctgaggagacggtgacc

SEQ ID NO: 41 - DNA, 23 bp, Artificial Sequence
  Description of Artificial Sequence Synthetic primer
ttagagagttgctttacgtggcc

SEQ ID NO: 42 - DNA, 20 bp, Artificial Sequence
  Description of Artificial Sequence Synthetic primer
cctggcttccttccctctgt

SEQ ID NO: 43 - DNA, 24 bp, Artificial Sequence
  Description of Artificial Sequence Synthetic probe
caggagggctctgggtgggtctgt

SEQ ID NO: 44 - DNA, 27 bp, Artificial Sequence
  Description of Artificial Sequence Synthetic probe
tgtccttcctttccactcctccccaga

SEQ ID NO: 45 - DNA, 19 bp, Artificial Sequence
  Description of Artificial Sequence Synthetic primer
acctgaggagacggtgacc

(19) United States
(12) Patent Application Publication
(10) Pub. No.: US 2010/0124741 A1
(43) Pub. Date: May 20, 2010

(54) METHODS FOR DETECTING IgH/BCL-1 CHROMOSOMAL TRANSLOCATION

(75) Inventor: Maher Albitar, Coto De Caza, CA (US)

Correspondence Address: FOLEY & LARDNER LLP, P.O. BOX 80278, SAN DIEGO, CA, 92138-0278, US

(73) Assignee: Quest Disgnostics Investments Incorporated

(21) Appl. No.: 12/496,570

(22) Filed: Jul. 1, 2009

Related U.S. Application Data
(60) Provisional application No. 61/115,873, filed on Nov. 18, 2008.

(51) Int. Cl. C12Q 1/68

(52) U.S. Cl. 435/6

(57) ABSTRACT

The invention provides methods for detection of Bcl-1 nucleic acid in acellular body fluid. The methods can be used to detect the IgH/Bcl-1 translocations (11;14)(q13;q32) in acellular body fluid. The chromosomal translocation (11;14)(q13;q32) is often associated with mantle cell (centrocytic) lymphoma and occasionally in other B-cell neoplasms, notably myeloma. The invention is useful in the diagnosis of mantle cell lymphoma (MCL) and also for determining the prognosis of the disease.

### CROSS REFERENCE TO RELATED PATENT APPLICATIONS

This application claims the priority of U.S. Patent Application No. 61/115,873, filed on Nov. 18, 2008. The prior filed application is incorporated herein by reference in its entirety.

### FIELD OF THE INVENTION

This invention relates to the field of cancer diagnosis. In particular, the invention relates to the diagnosis and prognosis of patients having myeloproliferative disease.

### BACKGROUND OF THE INVENTION

The following discussion of the background of the invention is merely provided to aid the reader in understanding the invention and is not admitted to describe or constitute prior art to the invention.

The Bcl-1 protein (also known as CCND1 or PRAD1) plays an important role in regulating the transition from G1 to S phase during the cell cycle (Lukas et al. Oncogene. 1994; 9: 2159-2167). Bcl-1 binds to and activates a cyclin-dependent kinase. The activated complex binds to and inactivates the retinoblastoma protein through phosphorylation, removing its inhibition of transcription factor E2F, which then leads to the transcription of DNA synthesis genes.

Rearrangement of the Bcl-1 (B-cell lymphoma 1) region on chromosome 11q13 appears to be highly characteristic of Mantle Cell Lymphoma (MCL) and also is found infrequently in other B-cell neoplasms such as B-cell chronic lymphocytic leukemia (B-CLL). The translocation involves rearrangement of chromosome 11q13 with the immunoglobulin heavy chain (IgH) locus on chromosome 14q32 (Erikson et al. Proc. Natl. Acad. Sci. USA 1984; 81: 4144-48; Rosenberg et al. Proc Natl. Acad. Sci USA. 1991; 88:9638-9642). The rearrangement of Bcl-1 deregulates the proto-oncogene. The translocation brings the Bcl-1 gene at 11q23 under the control of the immunoglobulin heavy chain gene on chromosome 14, resulting in an overexpression of Bcl-1. The Bcl-1 gene is transcriptionally silent in normal lymphohemopoietic tissues (Bartkova et al. J Pathol. 1994; 172; 237-245; Yang et al. Am J Pathol. 1994; 145: 86-96). An increase in Bcl-1 thereby shortens the time a cell spends in the resting G phase, and accelerates the transition into the S phase (Pines, J. Mammalian cell cycle control. Peters G, Vousden K H. editors. New York: Oxford University Press; Oncogenes and Tumour Suppressors. 1997:191-200) thus, expression of the protein may promote neoplastic cell proliferation by perpetuating the transition from G1 to S (Baldin et al. Genes Dev. 1993; 7: 812-21).

### SUMMARY OF THE INVENTION

This invention relates to detection of Bcl-1 nucleic acid in acellular body fluid. The invention is useful for detecting IgH/Bcl-1 chromosomal translocation in such fluid for prognosis and diagnosis relating to lymphoid malignancy.

In one aspect, the invention provides a method for determining the presence or absence of IgH/Bcl-1 chromosomal translocation in an individual, the method comprising: a) evaluating nucleic acid from an acellular bodily fluid sample of the individual to determine whether a portion of Bcl-1 nucleic acid is located in close proximity to a portion of IgH nucleic acid on a single polynucleotide; and b) identifying the individual as having IgH/Bcl-1 chromosomal translocation when a portion of Bcl-1 nucleic acid is in close proximity to a portion of IgH nucleic acid on a single polynucleotide.

In another aspect, the invention provides a method for diagnosing an individual as having lymphoid malignancy, the method comprising: a) providing an acellular bodily fluid sample from said individual; b) evaluating whether a portion of Bcl-1 nucleic acid is located in close proximity a portion of IgH nucleic acid on a single polynucleotide in the acellular body fluid sample; and c) identifying the individual as having lymphoid malignancy when a portion of Bcl-1 nucleic acid is in close proximity to a portion of IgH nucleic acid on a single polynucleotide.

In another aspect, the invention provides a method of determining a prognosis of an individual diagnosed with a lymphoid malignancy, the method comprising determining the presence or absence of IgH/Bcl-1 chromosomal translocation from an acellular bodily fluid of an individual, and identifying the patient as having poor prognosis, wherein the presence of IgH/Bcl-1 chromosomal translocation is indicative of poor prognosis.

In one embodiment of all aspects of the invention, the acellular body fluid is plasma or serum. In one embodiment, the portion of IgH nucleic acid is an enhancer. In one embodiment, the nucleic acid evaluated from the individual is genomic DNA. In another embodiment, the nucleic acid evaluated from the individual is mRNA.

In another embodiment of all aspects of the invention, the individual is diagnosed as having Mantle cell lymphoma (MCL). In another embodiment, the individual is diagnosed as having B-cell myeloma.

In another embodiment of all aspects of the invention, the method includes amplifying the nucleic acid from acellular body fluid using PCR. The PCR method may include using a PCR primer having the nucleotide sequence of SEQ ID NO: 38 and/or 40 or complements thereof. In some embodiments, the PCR method further uses a third primer and a fourth primer. In some embodiments, the method includes detecting the chromosomal translocation by hybridizing to the amplified nucleic acid a nucleic acid probe encompassing the junction and a first portion of the probe is specific for IgH nucleic acid and a second portion of the probe is specific for Bcl-1 nucleic acid. Suitable probes include for example, a probe having the sequence of SEQ ID NO: 39.

In another embodiment of all aspects of the invention, the method includes determining the presence or absence of the translocation using flow cytometry, or by determining the nucleotide sequence of the nucleic acid, or by determining the size of the nucleic acid. Size of a nucleic acid can be determined by several methods, for example, by HPLC, capillary electrophoresis, size exclusion chromatography, agarose gel electrophoresis.

In some embodiments, the method further includes determining the proportion of translocated Bcl-1 genomic nucleic acid relative to control nucleic acid in the acellular body fluid. In some embodiments, the control nucleic acid is wild-type Bcl-1 nucleic acid without any translocation. In another embodiment, the control nucleic acid is K-ras gene.

“Individual” as used herein means a human or any other animal which contains a Bcl-1 gene that can be amplified using the primers and methods described herein. An individual can be a patient, which refers to a human presenting to a medical provider for diagnosis or treatment of a disease. A human includes pre and post natal forms.

“Patient” as used herein refers to one who receives medical care, attention or treatment. As used herein, the term is meant to encompass a person diagnosed with a disease such as myeloproliferative disease as well as a person who may be symptomatic for a disease but who has not yet been diagnosed.

“Sample” or “patient sample” as used herein includes biological samples such as tissues and bodily fluids. “Bodily fluids” may include, but are not limited to, blood, serum, plasma, saliva, cerebral spinal fluid, pleural fluid, tears, lactal duct fluid, lymph, sputum, urine, amniotic fluid, and semen. A sample may include a bodily fluid that is “acellular.” An “acellular bodily fluid” includes less than about 1% (w/w) whole cellular material. Plasma or serum are examples of acellular bodily fluids. A sample may include a specimen of natural or synthetic origin.

“Plasma” as used herein refers to acellular fluid found in blood. “Plasma” may be obtained from blood by removing whole cellular material from blood by methods known in the art (e.g., centrifugation, filtration, and the like). As used herein, “peripheral blood plasma” refers to plasma obtained from peripheral blood samples.

“Serum” as used herein includes the fraction of plasma obtained after plasma or blood is permitted to clot and the clotted fraction is removed. “Nucleic acid” or “nucleic acid sequence” as used herein refers to an oligonucleotide, nucleotide or polynucleotide, and fragments or portions thereof, which may be single or double stranded, and represent the sense or antisense strand. A nucleic acid may include DNA or RNA, and may be of natural or synthetic origin and may contain deoxyribonucleotides, ribonucleotides, or nucleotide analogs in any combination.

Non-limiting examples of polynucleotides include a gene or gene fragment, genomic DNA, exons, introns, mRNA, tRNA, rRNA, ribozymes, cDNA, recombinant polynucleotides, branched polynucleotides, plasmids, vectors, isolated DNA of any sequence, isolated RNA of any sequence, synthetic nucleic acid, nucleic acid probes and primers. Polynucleotides may be natural or synthetic. Polynucleotide may comprise modified nucleotides, such as methylated nucleotides and nucleotide analogs, uracyl, other sugars and linking groups such as fluororibose and thiolate, and nucleotide branches. A nucleic acid may be modified such as by conjugation, with a labeling component. Other types of modifications included in this definition are caps, substitution of one or more of the naturally occurring nucleotides with an analog, and introduction of chemical entities for attaching the polynucleotide to other molecules such as proteins, metal ions, labeling components, other polynucleotides or a solid support. Nucleic acid may include nucleic acid that has been amplified (e.g., using polymerase chain reaction).

“Genomic nucleic acid” as used herein refers to the nucleic acid in a cell that is present in the cell chromosome(s) of an organism which contains the genes that encode the various proteins of the cells of that organism. A preferred type of genomic nucleic acid is that present in the nucleus of a eukaryotic cell. Genomic nucleic acid can be DNA or RNA. Genomic nucleic acid can be double stranded or single stranded, or partially double stranded, or partially single stranded or a hairpin molecule. Genomic nucleic acid may be intact or fragmented (e.g., digested with restriction endonucleases or by sonication or by applying shearing force by methods known in the art). In some cases, genomic nucleic acid may include sequence from all or a portion of a single gene or from multiple genes, sequence from one or more chromosomes, or sequence from all chromosomes of a cell. As is well known, genomic nucleic acid includes gene coding regions, introns, 5′ and 3′ untranslated regions, 5′ and 3′ flanking DNA and structural segments such as telomeric and centromeric DNA, replication origins, and intergenic DNA. Genomic nucleic acid representing the total nucleic acid of the genome is referred to as “total genomic nucleic acid.”

Genomic nucleic acid may be obtained by methods of extraction/purification from acellular body fluids as is well known in the art. The ultimate source of genomic nucleic acid can be normal cells or may be cells that contain one or more mutations in the genomic nucleic acid, e.g., duplication, deletion, translocation, and transversion. Included in the meaning of genomic nucleic acid is genomic nucleic acid that has been subjected to an amplification step that increases the amount of the target sequence of interest sought to be detected relative to other nucleic acid sequences in the genomic nucleic acid.

A fragment of a nucleic acid generally contains at least about 15, 20, 25, 30, 35, 40, 45, 50, 75, 100, 200, 300, 400, 500, 1000 nucleotides or more. Larger fragments are possible and may include about 2,000, 2,500, 3,000, 3,500, 4,000, 5,000 7,500, 10,000, 20,000, 50,000, 100,000 bases or more.

“A portion of” in the context of a nucleic acid refers to a sequence of nucleotide residues which are at least about 10 nucleotides, at least about 20 nucleotides, at least about 25 nucleotides, at least about 30 nucleotides, at least about 40 nucleotides, at least about 50 nucleotides, at least about 100 nucleotides, at least about 250 nucleotides, at least about 500 nucleotides, at least about 1,000 nucleotides, at least about 2,000 nucleotides, at least about 5,000 nucleotides, at least about 10,000 nucleotides, at least about 20,000 nucleotides, at least about 50,000 nucleotides, at least about 100,000 nucleotides, at least about 500,000 nucleotides, at least about 1,000,000 nucleotides or more. A portion of Bcl-1 and IgH nucleic acids includes both coding and non-coding regions. Exemplary non-coding region includes but not limited to 5′-untranslated regions (e.g., promoters, enhancers), introns, and 3′-untranslated regions.

“Close proximity” in the context of IgH/Bcl-1 chromosomal translocation means when a portion of Bcl-1 nucleic acid is directly joined to a portion of IgH nucleic acid on a single polynucleotide, or when portions of Bcl-1 and IgH nucleic acids are separated from each other on a single polynucleotide by less than about: 2, 3, 4, 5, 6, 7, 8, 9, 10, 11, 12, 13, 14, 15, 16, 17, 18, 19, 20, 21, 22, 23, 24, 25, 26, 27, 28, 29 , 30, 40, 50, 60, 70, 80, 90, or 100 nucleotides. Nucleotides separating portions of Bcl-1 nucleic acid and IgH nucleic acids may be non-homologous to chromosome 11 or chromosome 14 or both.

“Identity” and “identical” as used herein refer to a degree of identity between sequences. There may be partial identity or complete identity. A partially identical sequence is one that is less than 100% identical to another sequence. Preferably, partially identical sequences have an overall identity of at least 70% or at least 75%, more preferably at least 80% or at least 85%, most preferably at least 90% or at least 95% or at least 99%. Sequence identity determinations may be made for sequences which are not fully aligned. In such instances, the most related segments may he aligned for optimal sequence identity by and the overall sequence identity reduced by a penalty for gaps in the alignment.

“Substantially all” as used herein means between about 60%, about 70%, about 80%, about 90%, about 95%, about 99% or 100%.

“Substantially pure” as used herein in the context of nucleic acid represents at least 50%, at least 60%, at least 70%, at least 80%, at least 90%, at least 95% or at least 99% of the nucleic acid in a sample. The nucleic acid sample may exist in solution or as a dry preparation.

“Isolated” as used herein when referring to a nucleic acid (e.g., an oligonucleotide such as RNA, DNA, or a mixed polymer) means a nucleic acid that is apart from a substantial portion of the genome in which it naturally occurs and/or is substantially separated from other cellular components which naturally accompany such nucleic acid. For example, any nucleic acid that has been produced synthetically (e.g., by serial base condensation) is considered to be isolated. Likewise, nucleic acids that are recombinantly expressed, cloned, produced by a primer extension reaction (e.g., PCR), or otherwise excised from a genome are also considered to be isolated.

“Specific hybridization” as used herein is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC. Stringency of hybridization may be expressed, in part, with reference to the temperature under which the wash steps are carried out. Such temperatures are typically selected to he about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Equations for calculating Tm and conditions for nucleic acid hybridization are known in the art. “Stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5×Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

“Substantially complementary” as used herein means that two sequences hybridize under stringent hybridization conditions. The skilled artisan will understand that substantially complementary sequences need not hybridize along their entire length. Oligonucleotides can be used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

“Oligonucleotide” as used herein refers to a molecule that has a sequence of nucleic acid bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group in this position. Oligonucleotides also may include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, reverse transcription, PCR, or a combination thereof. The oligonucleotide may be modified. For example, the oligonucleotide may be labeled with an agent that produces a detectable signal (e.g., a fluorophore).

“Primer” as used herein refers to an oligonucleotide that is capable of acting as a point of initiation of synthesis when placed under conditions in which primer extension is initiated (e.g., primer extension associated with an application such as PCR). The primer is complementary to a target nucleotide sequence and it hybridizes to a substantially complementary sequence in the target and leads to addition of nucleotides to the 3′-end of the primer in the presence of a DNA or RNA polymerase. The 3′-nucleotide of the primer should generally be complementary to the target sequence at a corresponding nucleotide position for optimal expression and amplification. An oligonucleotide “primer” may occur naturally, as in a purified restriction digest or may be produced synthetically. The term “primer” as used herein includes all forms of primers that may be synthesized including peptide nucleic acid primers, locked nucleic acid primers, phosphorothioate modified primers, labeled primers, and the like.

Primers are typically between about 10 and about 100 nucleotides in length, preferably between about 15 and about 60 nucleotides in length, more preferably between about 20 and about 50 nucleotides in length, and most preferably between about 25 and about 40 nucleotides in length. In some embodiments, primers can be at least 8, at least 12, at least 16, at least 20, at least 25, at least 30, at least 35, at least 40, at least 45, at least 50, at least 55, at least 60 nucleotides in length. An optimal length for a particular primer application may be readily determined in the manner described in H. Erlich, PCR Technology, Principles and Application for DNA Amplification (1989).

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid.

Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be fully complementary to a target nucleic acid sequence or partially complementary. A probe may be used to detect the presence or absence of a target nucleic acid. Probes are typically at least about 10, 15, 20, 25, 30, 35, 40, 50, 60, 75, 100 nucleotides or more in length.

“Target nucleic acid” as used herein refers to a nucleic acid molecule (e.g., DNA or RNA) containing a sequence that has at least partial complementarity with a primer oligonucleotide and/or a probe oligonucleotide. A probe may specifically hybridize to a target nucleic acid.

“Assay” or “assaying” as used herein means qualitative or quantitative analysis or testing.

“Detectable label” as used herein refers to a molecule or a compound or a group of molecules or a group of compounds used to identify a nucleic acid or a protein of interest. In some cases, the detectable label may be detected directly. In other cases, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable label include but are not limited to spectroscopic, photochemical, biochemical, immunochemical, electromagnetic, radiochemical, or chemical means, such as fluorescence, chemifluoresence, or chemiluminescence, or any other appropriate means.

“About” as used herein means in quantitative terms, plus or minus 10%.

“Ratio” as used herein refers to the relation in degree or number between two similar things. For example, the relative amount of translocated Bcl-1 nucleic acid to wild-type Bcl-1 nucleic acid in a sample may be referred to as a ratio of wild-type to translocated Bcl-1 nucleic acid.

“Diagnose” or “diagnosis” or “diagnosing” as used herein refer to distinguishing or identifying a disease, syndrome or condition or distinguishing or identifying a person having a particular disease, syndrome or condition. Usually, a diagnosis of a disease or disorder is based on the evaluation of one or more factors and/or symptoms that are indicative of the disease. That is, a diagnosis can be made based on the presence, absence or amount of a factor which is indicative of presence or absence of the disease or condition. Each factor or symptom that is considered to be indicative for the diagnosis of a particular disease does not need be exclusively related to the particular disease; i.e. there may be differential diagnoses that can be inferred from a diagnostic factor or symptom. Likewise, there may he instances where a factor or symptom that is indicative of a particular disease is present in an individual that does not have the particular disease.

“Treatment,” “treating,” or “treat” as used herein refers to care by procedures or application that are intended to relieve illness or injury. Although it is preferred that treating a condition or disease will result in an improvement of the condition, the term treating as used herein does not indicate, imply, or require that the procedures or applications are at all successful in ameliorating symptoms associated with any particular condition. Treating a patient may result in adverse side effects or even a worsening of the condition which the treatment was intended to improve.

The term “prognosis” as used herein refers to a prediction of the probable course and outcome of a clinical condition or disease. A prognosis of a patient is usually made by evaluating factors or symptoms of a disease that are indicative of a favorable or unfavorable course or outcome of the disease.

The phrase “determining the prognosis” as used herein refers to the process by which the skilled artisan can predict the course or outcome of a condition in a patient. The term “prognosis” does not refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition. A prognosis may be expressed as the amount of time a patient can be expected to survive. Alternatively, a prognosis may refer to the likelihood that the disease goes into remission or to the amount of time the disease can be expected to remain in remission. Prognosis can be expressed in various ways; for example prognosis can be expressed as a percent chance that a patient will survive after one year, five years, ten years or the like. Alternatively prognosis may be expressed as the number of years, on average that a patient can expect to survive as a result of a condition or disease. The prognosis of a patient may be considered as an expression of relativism, with many factors effecting the ultimate outcome. For example, for patients with certain conditions, prognosis can be appropriately expressed as the likelihood that a condition may be treatable or curable, or the likelihood that a disease will go into remission, whereas for patients with more severe conditions prognosis may be more appropriately expressed as likelihood of survival for a specified period of time.

The term “poor prognosis” as used herein, in the context of a patient having an IgH/Bcl-1 chromosomal translocation, refers to an increased likelihood that the patient will have a worse outcome in a clinical condition relative to a patient diagnosed as having the same disease but lacking the IgH/Bcl-1 chromosomal translocation. A poor prognosis may be expressed in any relevant prognostic terms and may include, for example, the expectation of a reduced duration of remission, reduced survival rate, and reduced survival duration.

### BRIEF DESCRIPTION OF THE FIGURES

FIG. 1 shows an exemplary genomic sequence of human Bcl-1 gene (SEQ ID NO: 1).

FIG. 2 shows an exemplary mRNA sequence of human Bcl-1 (SEQ ID NO: 2)

FIG. 3 shows a schematic diagram of the Bcl-1 gene and the position of break point regions in chromosome 11, the major translocation cluster (MTC) and two minor translocation clusters TC1 and TC2. The solid box indicates Bcl-1 gene. The orientation of the Bcl-1 gene on chromosome 11 with respect to the centromere (CEN) and the telomere (TEL) are indicated.

FIG. 4 shows exemplary sequences associated with IgH/Bcl-1 translocation. FIG. 4A-4S shows the nucleic acid sequence encompassing the break point junction of chromosome 11 and 14 in t(11;14)(q13;q32) translocation (SEQ ID NO: 5-23). Sequence of chromosome 11 is shown in uppercase. N region insertions sequences (stretches of DNA inserted during IgH/Bcl-1 translocation) are shown in uppercase and underlined. Sequence of chromosome 14 is shown in lowercase. FIG. 4T-4U shows exemplary sequences of Bcl-1 locus associated with t(11;14)(q13;q32) breakpoint junction (SEQ ID NO: 24-25).

FIG. 5 shows the results of the analysis of the PCR amplified fragments of IgH and TCR-γ gene by capillary gel electrophoresis (CGE). Representative CGE profiles of matched peripheral blood and plasma samples are shown. The expected product sizes ranged from 220-310 bp for IgH and 140-180 bp for TCR-γ respectively.

FIG. 6 shows an exemplary genomic sequence of human K-ras gene (SEQ ID NO: 26).

### DETAILED DESCRIPTION OF THE INVENTION

The present invention provides methods for detection of Bcl-1 nucleic acid in acellular body fluid. Exemplary Bcl-1 nucleic acid includes but is not limited to genomic DNA, mRNA, and cDNA derived from mRNA. The methods can be used to detect the translocations of Bcl-1 gene (11;14)(q13;q32) in chromosome 11q13 with the immunoglobulin heavy chain locus on chromosome 14q32 in acellular body fluid. The chromosomal translocation (11;14)(q13;q32) is often associated with myeloproliferative disease such as mantle cell (centrocytic) lymphoma and occasionally with other B-cell neoplasms, notably myeloma. The methods are useful in the diagnosis of mantle cell lymphoma (MCL) and also for determining a prognosis for a patient with the disease.

Bcl-1 Nucleic Acid

Bcl-1 nucleic acid detected by the methods of the invention may be intact or fragmented and also may be double or single stranded. In some embodiments, Bcl-1 nucleic acid is partially double stranded. In one embodiment, Bcl-1 nucleic acid comprises a translated region. In another embodiment, Bcl-1 nucleic acid may include a untranslated region. Non-limiting examples of untranslated region include introns, 5′-untranslated region (5′-UTR), 3′-untranslated region (3′-UTR). In some embodiments. Bcl-1 nucleic acid includes both translated and untranslated regions.

Human Bcl-1 gene (also known as CCND1, PRAD1, U21B31) is located in human chromosome 11q13. Exemplary sequence of human chromosome 11 includes but is not limited to GenBank Accession numbers: NW_{—}001838027, NC_{—}000011, NT_{—}078088, AC_{—}000054, NW_{—}925106, AP001824, AP001888. Sequences of human chromosome 11 have also been reported previously (International Human Genome Sequencing Consortium. Nature 2004; 431: 931-945). These sequences are incorporated herein by reference.

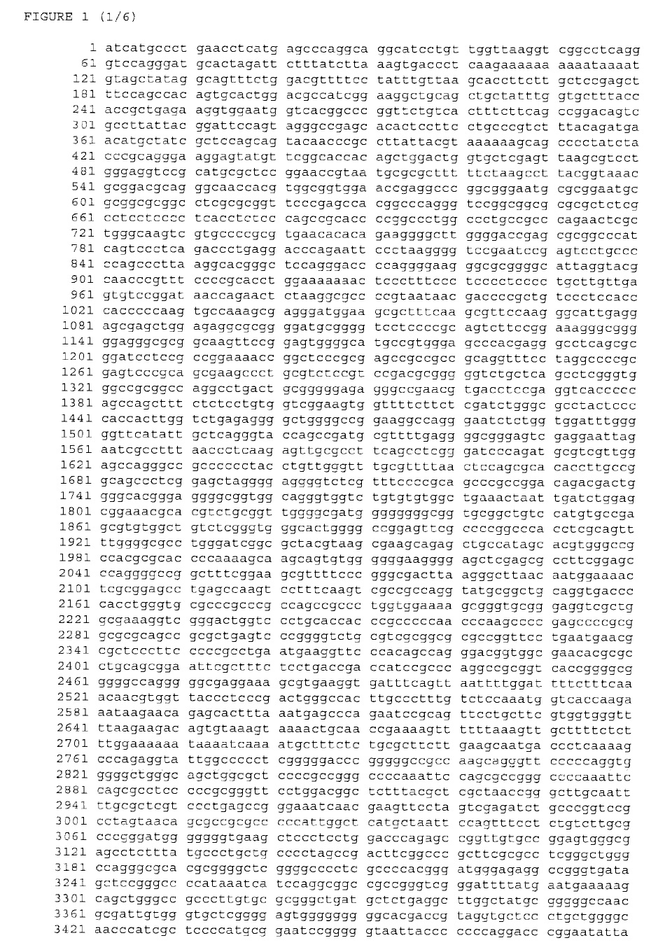

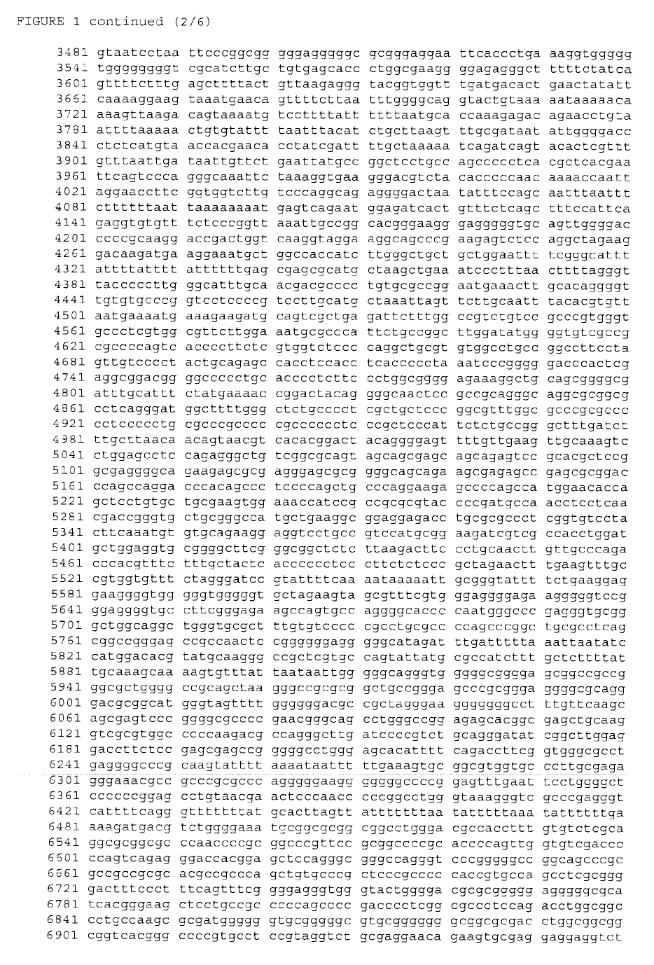

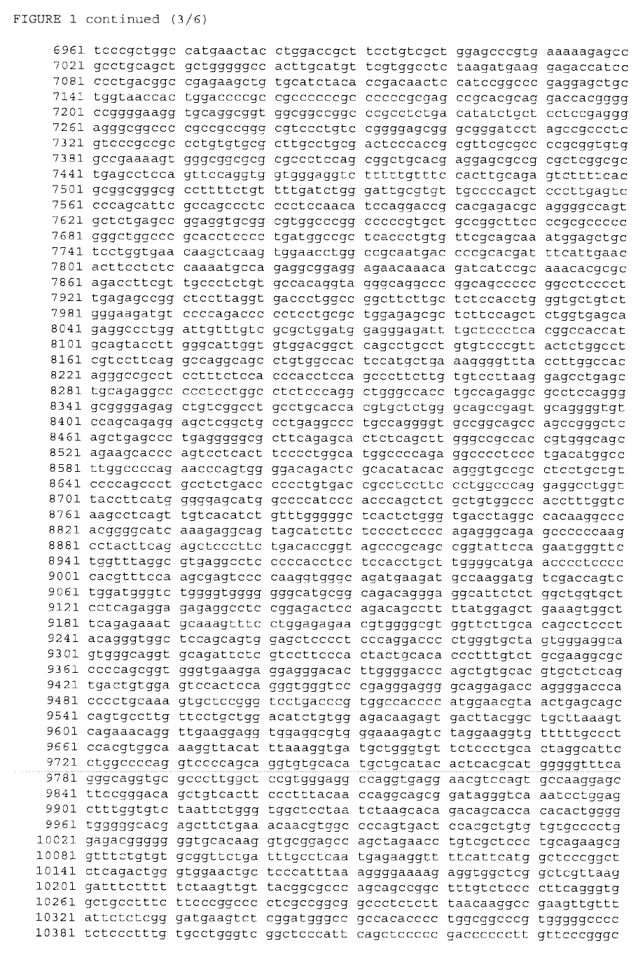

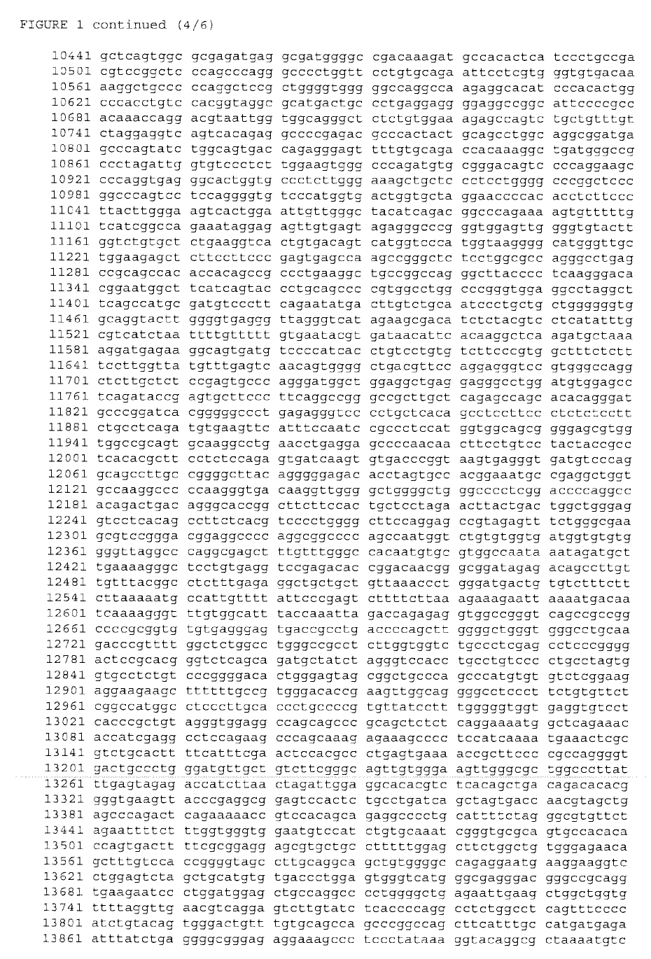

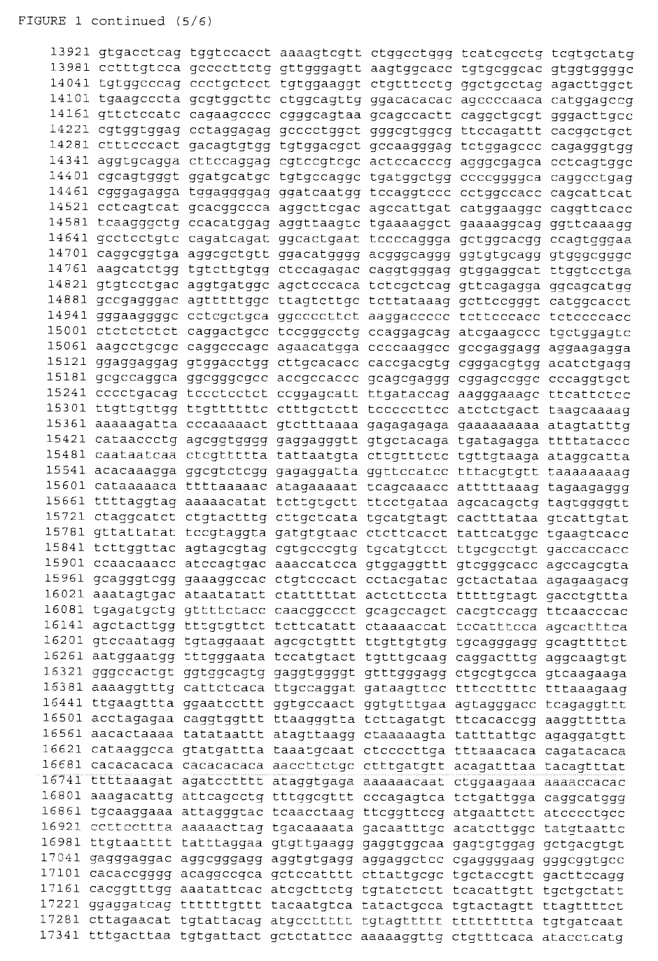

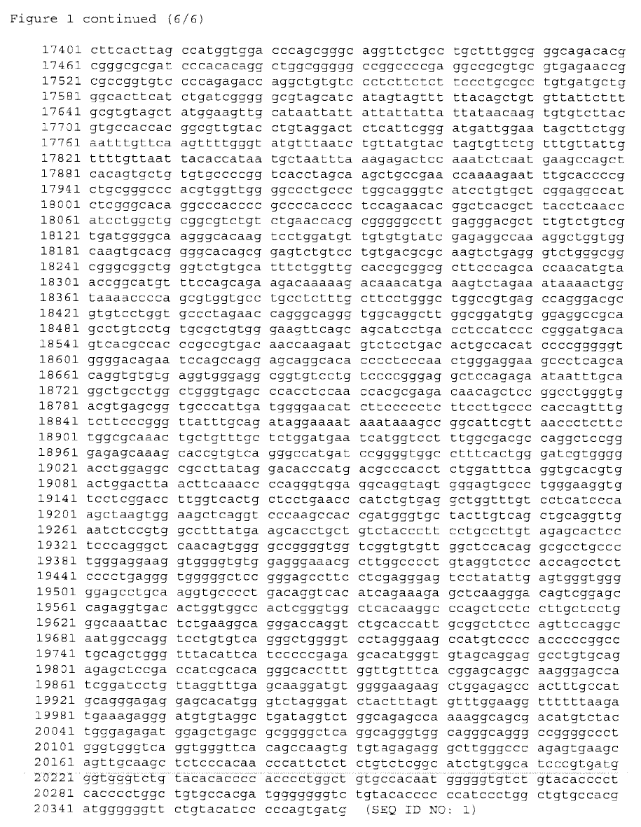

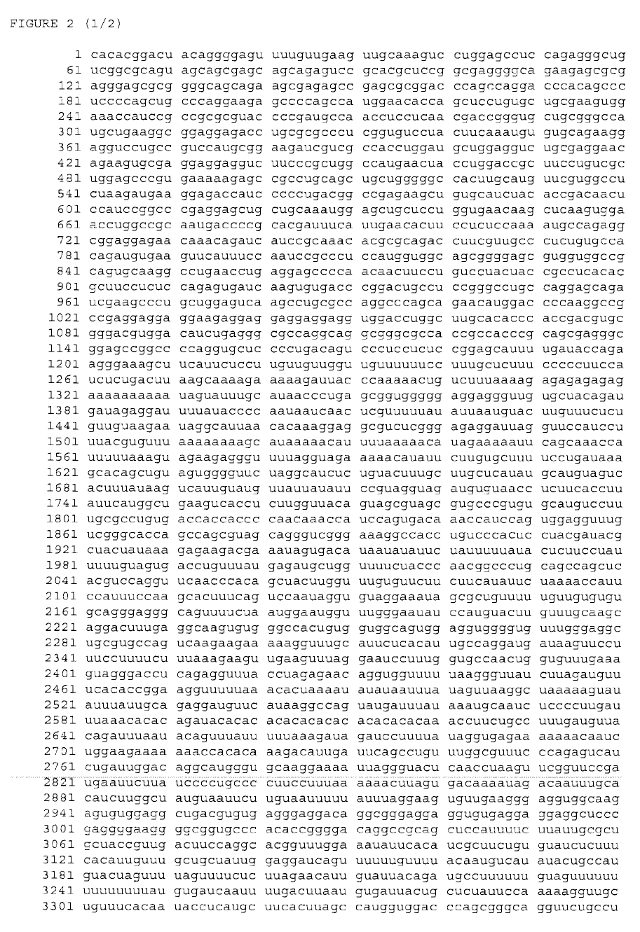

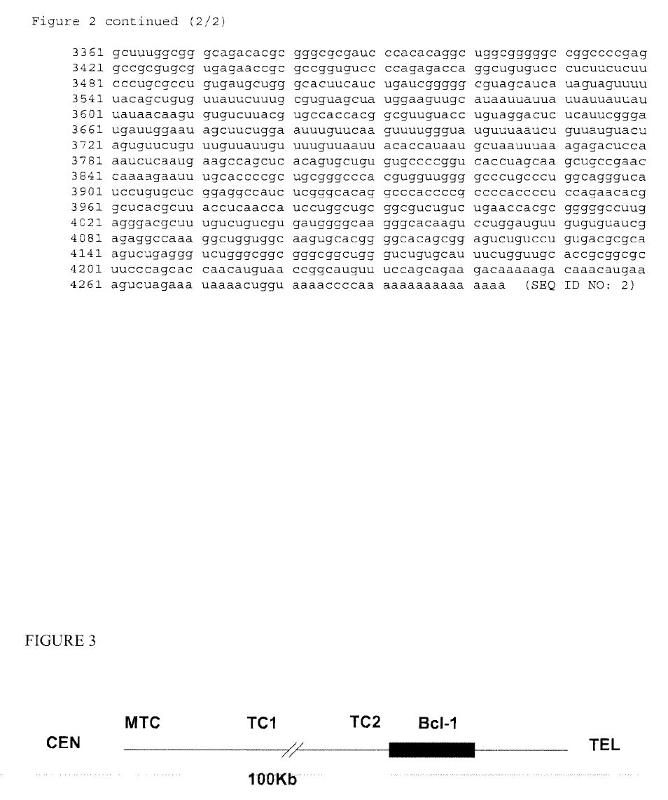

Exemplary Bcl-1 genomic DNA sequences include but are not limited to: NCBI GenBank accession numbers: NG_{—}007375, AF511593, CH471076. These sequences are incorporated herein by reference. Exemplary sequence of Bcl-1 gene is listed as SEQ ID NO: 1 and shown in FIG. 1. Partial and full length sequences of Bcl-1 mRNA are known in the art. Exemplary Bcl-1 mRNA sequences include but are not limited to: NCBI GenBank accession numbers: NM_{—}053056, X59798, Z23022, AY830112, AK299044, AK313136, AY830112, BC000076, BC001501, BC014078, BC023620, BC025302, BM796500, BT019844, BT019845, CR536538, CR542099, CR602163, CR605397, CR624763, M64349, M73554, M74092, X59798. All of these Bcl-1 mRNA sequences are incorporated herein by reference. Exemplary sequence of Bcl-1 mRNA is listed as SEQ ID NO: 2 and shown in FIG. 2. Exemplary Bcl-1 promoter sequences include but are not limited to: NCBI GenBank accession numbers: Z29078, L09054. Sequences of which are incorporated herein by reference.

IgH/Bcl-1 Translocation

The t(11;14)(q13;q32) translocation is an important abnormality associated with B-lymphocytic malignancy (Fukuhara et al. Cancer Res. 1979; 39: 3119-3128; Nishida et al. Cancer Res. 1989; 49: 1275-1281). Breakpoints at chromosome 14q32 occur in the joining region of the immunoglobulin heavy-chain (IgH) gene (Meeker et al. Blood. 1989; 74: 1801-1806; Tsujimoto et al. Nature (London). 1986; 315: 340-343). Chromosome 11 breakpoints occur in a region called the Bcl-1 (B-cell leukemia/lymphoma 1) locus, covering at least 63 kb of chromosome 11 (Tsujimoto et al. Nature (London). 1986; 315: 340-343; Koduru et al. Oncogene. 1989; 4: 929-934; Meeker et al. Blood. 1989; 74: 1801-1806). A high proportion of the documented breakpoints in chromosome 11 are found in a subregion of the locus called the major translocation cluster (MTC). Two minor translocation clusters in chromosome 11, TC1 and TC2 have been reported (Rimokh et al. Blood. 1993; 81: 3063-67). A schematic diagram of the Bcl-1 gene and the position of break point regions in chromosome 11, the MTC and two minor translocation clusters TC1 and TC2 are shown in FIG. 3.

In one embodiment. a portion of the MTC in chromosome 11 is SEQ ID NO: 3 and shown below:

(SEQ ID NO: 3)

5′-GATGAGATTAAACTGCGTCTTCTTCGTGGTTTGAACGCAAGAGCTCC
CTGAACACCCTGGCGC-3′

In another embodiment, a portion of the MTC in chromosome 11 is SEQ ID NO: 4 and shown below:

(SEQ ID NO: 4)

5′-TCTAGAATGT CAAAGAGTTG GACTCATACG GTGTGTAGCC
TTTTCAGACG GGCTTCTCTC ACCTACTAAT ATCGTGGAAG
TTTCCTCCAT ATCTTTTCAG GCCTTGATAG CTCGTGTCTT
TTTAGCGCTG AATAATATTG CACTGTCTGG ATGCACCGCG
GCTCAACCCT TCACCTACTG AAGGACTTGT GGGTTGCTTC
CAAGTTTTGG TCATTATGAA TAAGGCTGCT GTACACATCG
GTGTGCAGGT TTTTGCGTGG ACGTCTCAAC TCCTTTGGAT
AAAGGCGAGG AGCATAATTG CTGCACTGCA TATTCGGTTA
GACTGTGATT AGCTTTCTAA AAAGTGGTTT TGTTAGATGT
AAAAAATGAA TATGACATTC TGAAACAGAA AAAAATAACT
TACTCTTTAT CTGAGTGGGA TGAGATTAAA CTGCGTCTTC
TTCGTGGTTT GAACGCAAGA GCTCCCTGAA CACCTGGCGC
TGCCATTGGC GTGAACGAGG GGAAGCCCCT CCTGACAGCT
GGATGGTAGG ACAAAGCCCT CTAAGCCCCC TCTCCCCGTC
ACATCCCCCC GACCCTGCCC ACAAGGGAAC CTGGGGCACT
GGGTGTTCAC CTGCCTCCCA CTAGGTGAGA TCT-3′

In the germline state, the immunoglobulin heavy chain (IgH) gene exists as discontinuous gene segments coding for the variable (VH), divesity (D), joining (JH) and constant (CH) regions of the heavy chain proteins. Exemplary sequences of human IgH include but are not limited to NCBI GenBank accession numbers: EU687211, EU687210, EU687209, EU687208, EU687207, EU687206, EU687205, EU687204, EU687203, EU687202, EU687201, EU687200. These sequences are incorporated herein by reference. In some embodiments, the breakpoints in the chromosome 14 occur in the joining segments of the IgH locus. In some embodiments, the joining segments of IgH involved in the translocation may include J1, J2, J4 or J6.

In some embodiments, nucleic acid comprising the IgH/Bcl-1 translocation may further comprise an insertion of nucleic acid sequence of variable length between the junction of clearly identifiable chromosome 11 and 14 portions of each sequence. The insertion sequences are non-homologous to chromosome 14.

Sequences encompassing the IgH/Bcl-1 breakpoint junction have been reported. Exemplary sequences include but are not limited to NCBI GenBank accession numbers: DQ400340, DQ912821, DQ401134, DQ241758, AF018254, Y11644, Y11645, U73667, AF230880, U73664. Exemplary Bcl-1 locus sequence in chromosome 11 associated with translocation include but are not limited to NCBI GenBank accession numbers: U73666, U73665 These sequences are incorporated herein by reference.

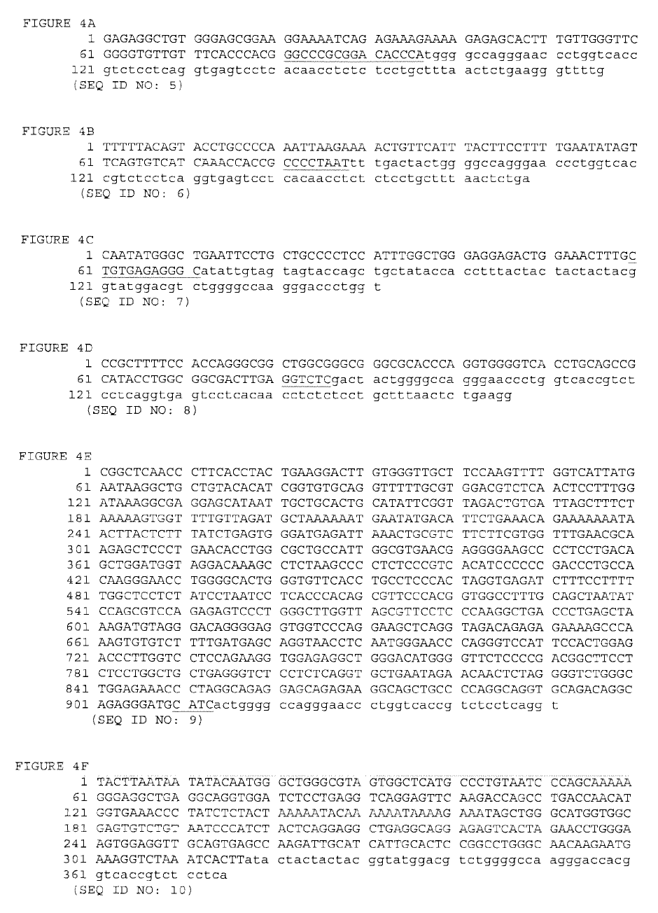

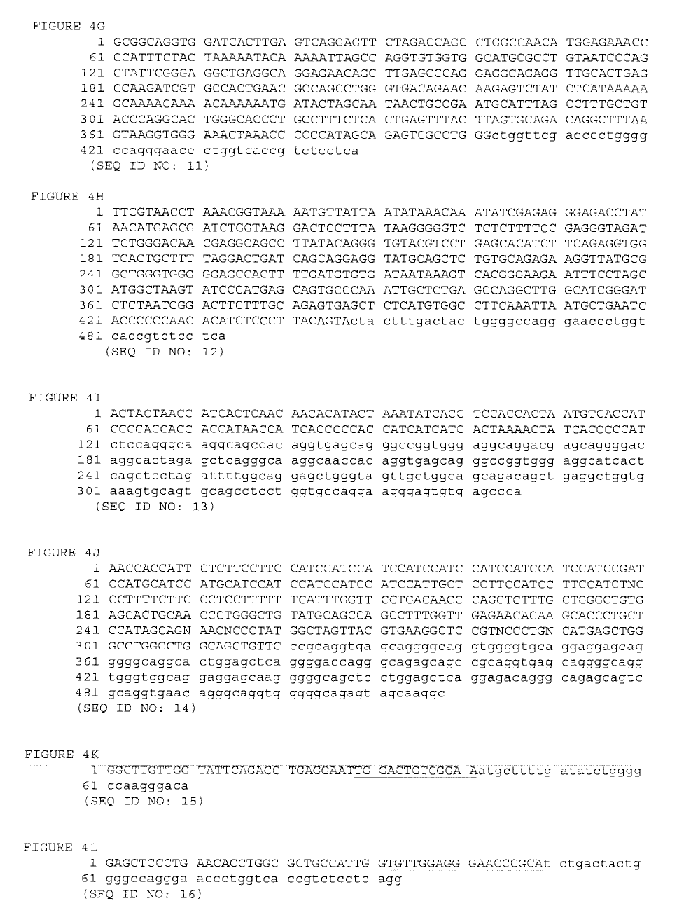

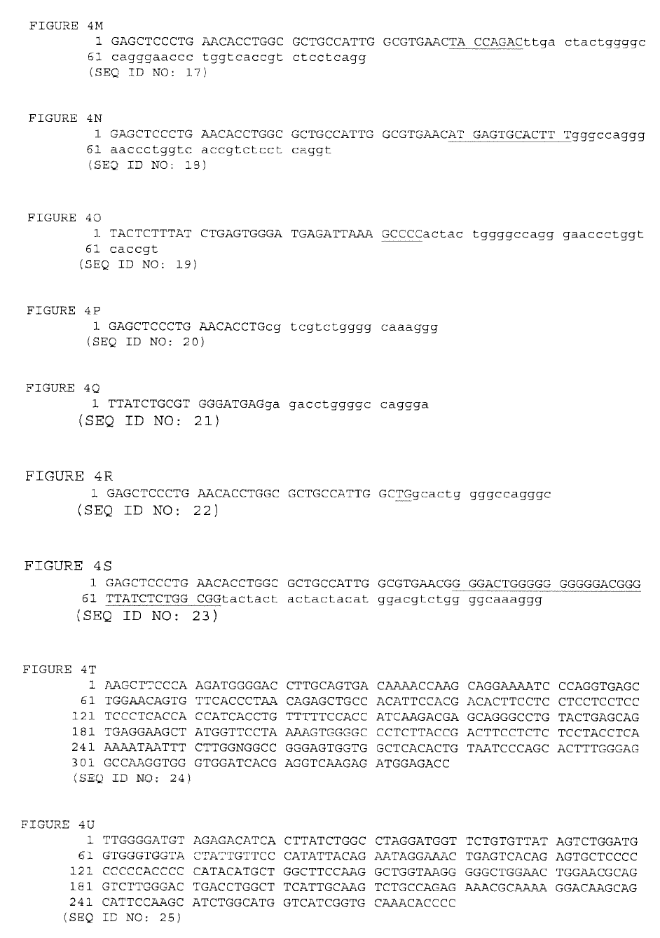

In some embodiments, the sequence encompassing IgH/Bcl-1 breakpoint junction may be any of the sequences listed as SEQ ID NO: 5-23 and shown in FIGS. 4A-4S respectively. In some embodiments, the Bcl-1 locus sequence in chromosome 11 associated with translocation may be any of SEQ ID NO: 24-25 and shown in FIG. 4T-4U respectively.

In some embodiments, the IgH/Bcl-1 translocation may be detected by PCR. In one embodiment, the PCR method may include a primer pair where one primer of the primer pair hybridizes to a portion of IgH J region and the other primer of the primer pair hybridizes to a portion of Bcl-1 locus. In one embodiment, the primer hybridizes to MTC portion. In one embodiment, the primers used to detect the IgH/Bcl-1 translocation in a PCR reaction may be SEQ ID NO: 38 and SEQ ID NO: 40. In one embodiment, the primer SEQ ID NO: 38 may hybridize to a portion of a sequence disclosed in GenBank accession number NW_{—}001838027. In one embodiment, the primer SEQ ID NO: 40 may hybridize to a portion of a sequence disclosed in GenBank accession number NW_{—}001838121. In some embodiments, the IgH/Bcl-1 translocation may be detected by southern blot. In another embodiment, the IgH/Bcl-1 translocation may be detected by fluorescent in situ hybridization. In some embodiments the IgH/Bcl-1 translocation may be detected by flow cytometry. In another embodiment, the IgH/Bcl-1 translocation may be detected by nucleic acid sequencing. In one embodiment, the IgH/Bcl-1 translocation may be detected by size. In some embodiments the detection of IgH/Bcl-1 translocation may also include detection of an internal control.

In one embodiment, the IgH/Bcl-1 translocation may be detected by hybridization of a nucleic acid probe to genomic DNA or to a portion of amplified genomic DNA comprising the translocation. In some embodiments, the probe may encompass the junction and a first portion of the probe is specific for IgH nucleic acid and a second portion of the probe is specific for Bcl-1 nucleic acid.

In one embodiment, the IgH/Bcl-1 translocation may be detected by real time PCR using TaqMan® probes. In one embodiment, the primers used to detect the IgH/Bcl-1 translocation in a real time PCR reaction may be SEQ ID NO: 38 and SEQ ID NO: 40. In one embodiment, the TaqMan® probe used to detect the IgH/Bcl-1 translocation in a real time PCR reaction may be SEQ ID NO: 39. In one embodiment, the primer SEQ ID NO: 38 and the probe SEQ ID NO: 39 may hybridize to a portion of a sequence disclosed in GenBank accession number NW_{—}001838027. In one embodiment, the primer SEQ ID NO: 40 may hybridize to a portion of a sequence disclosed in GenBank accession number NW_{—}001838121.

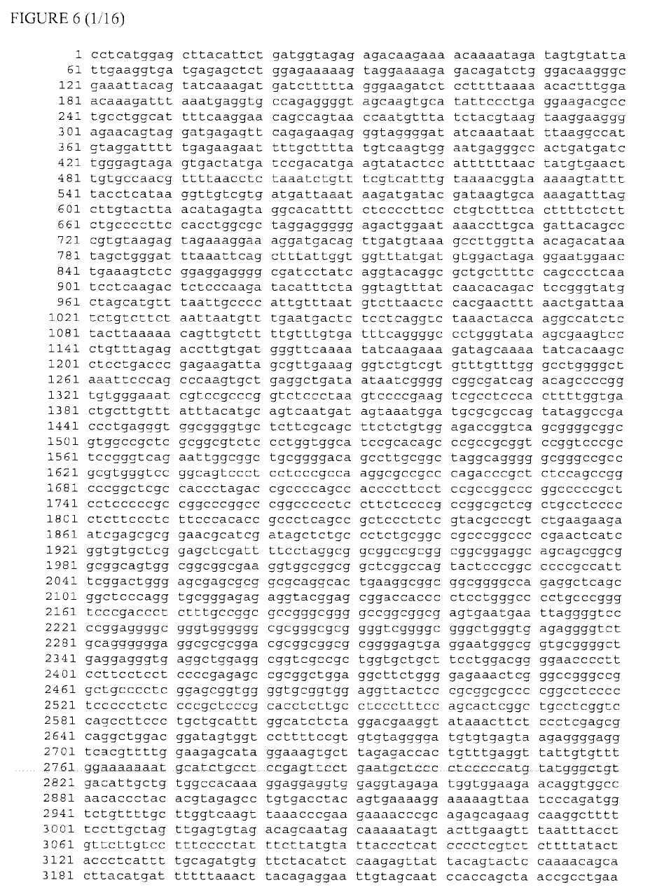

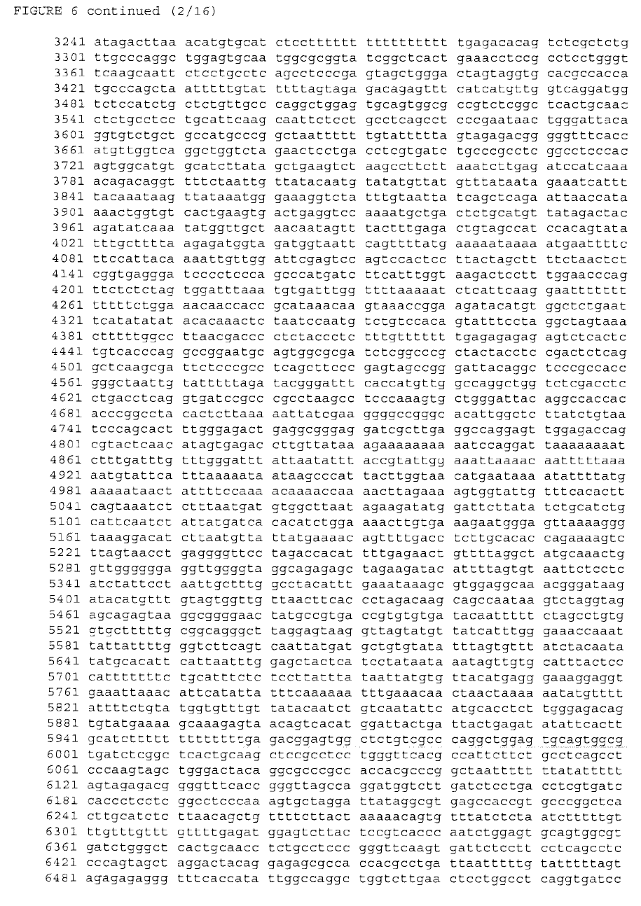

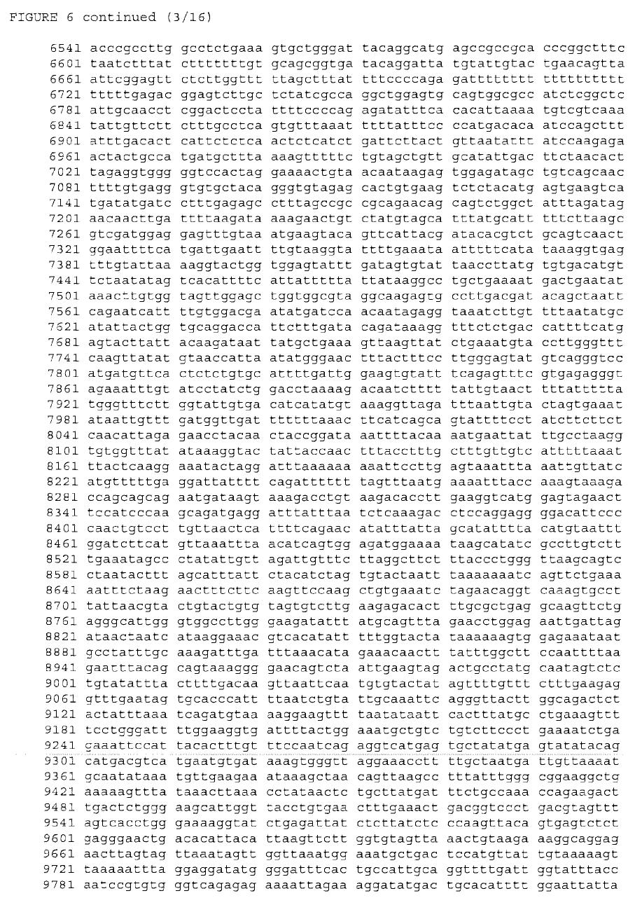

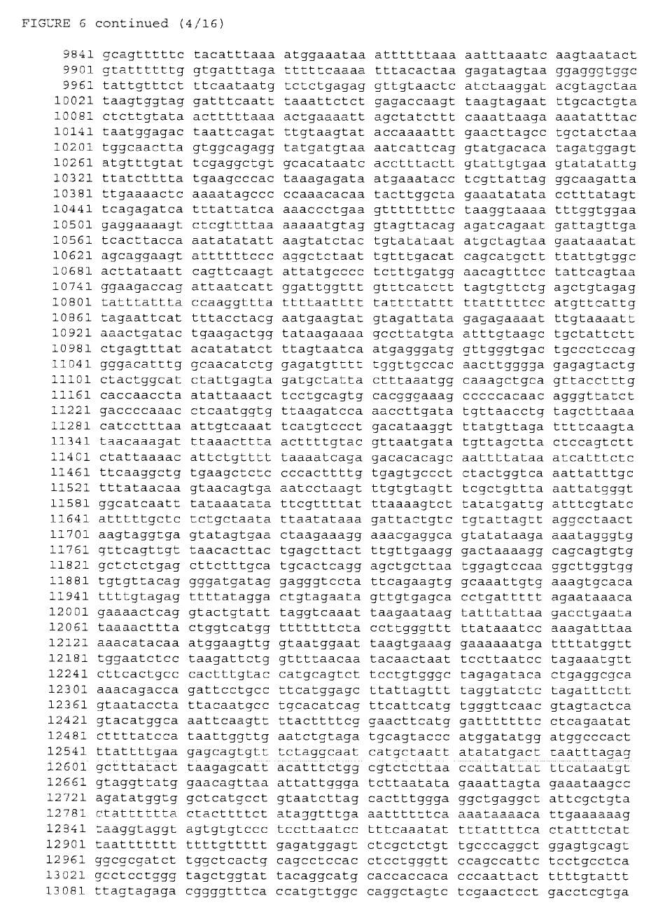

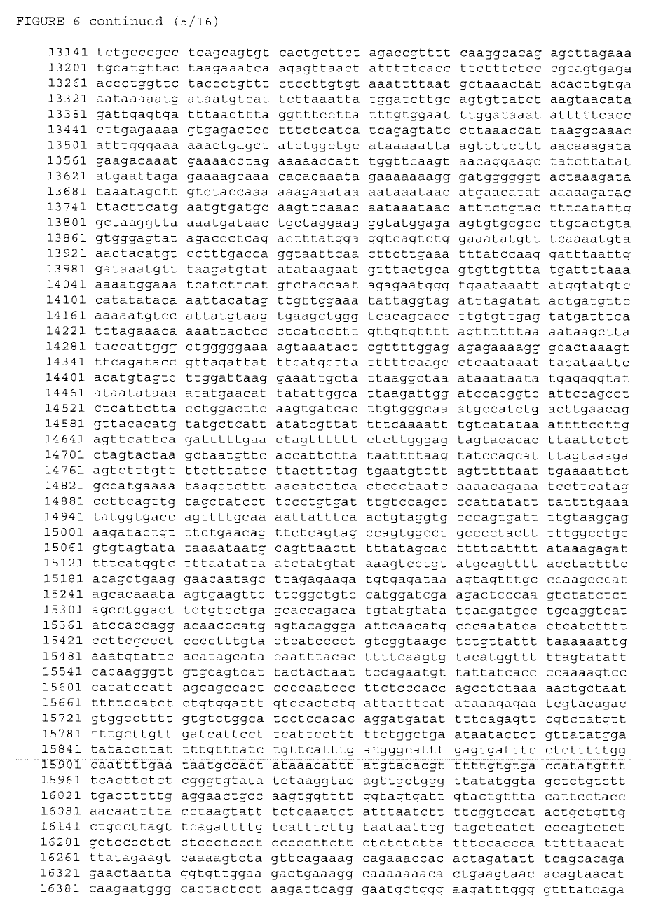

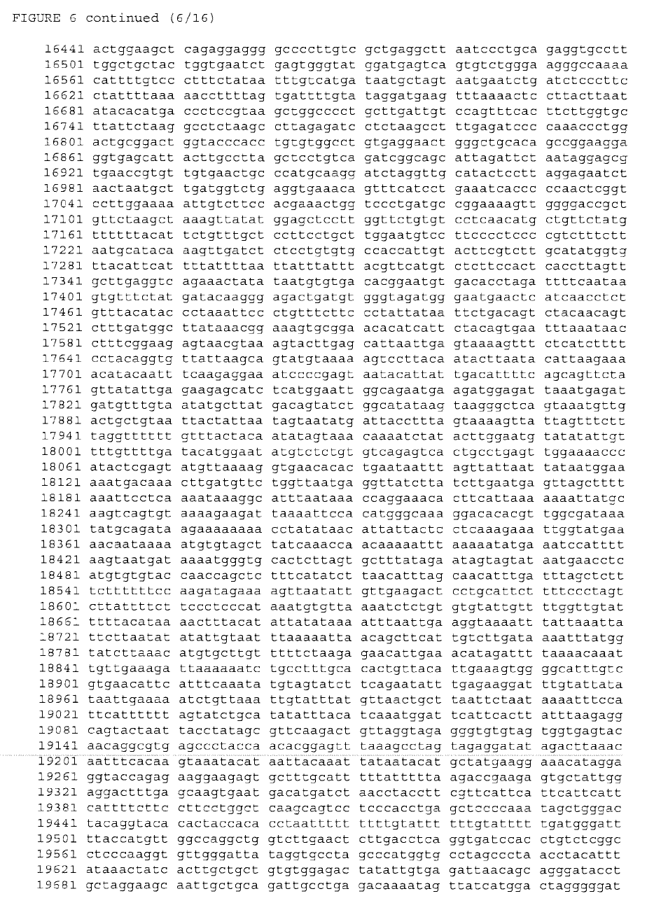

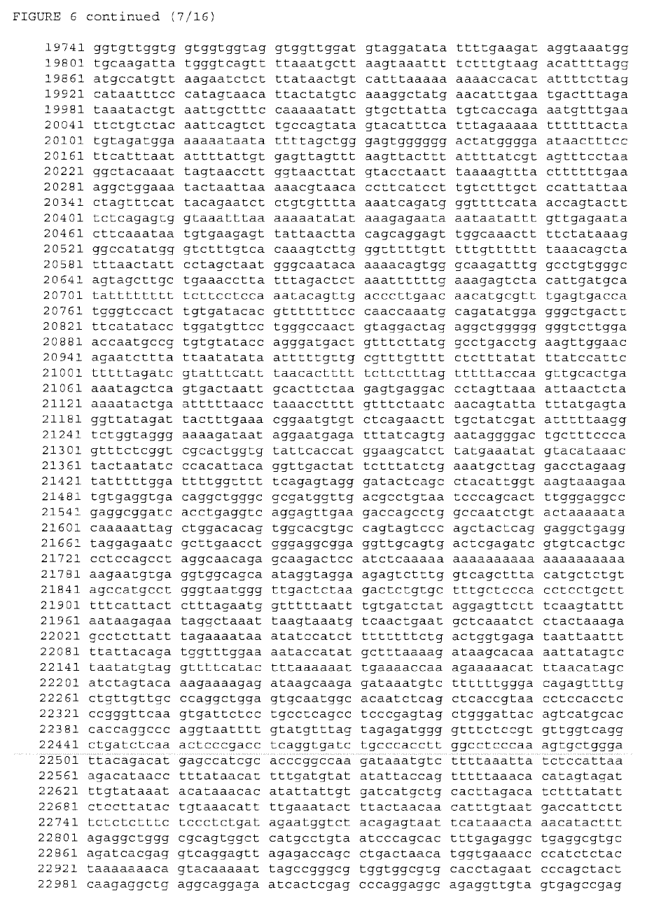

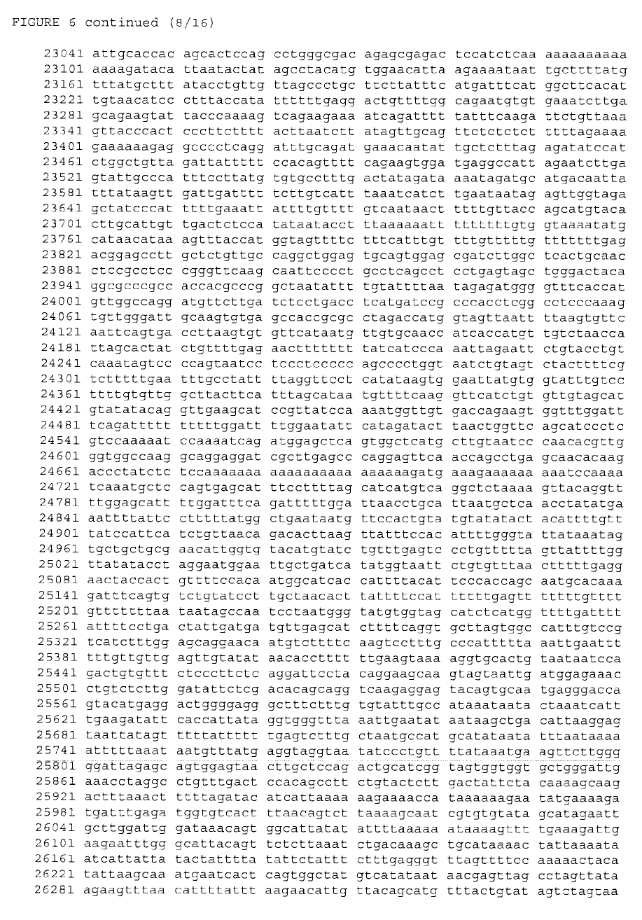

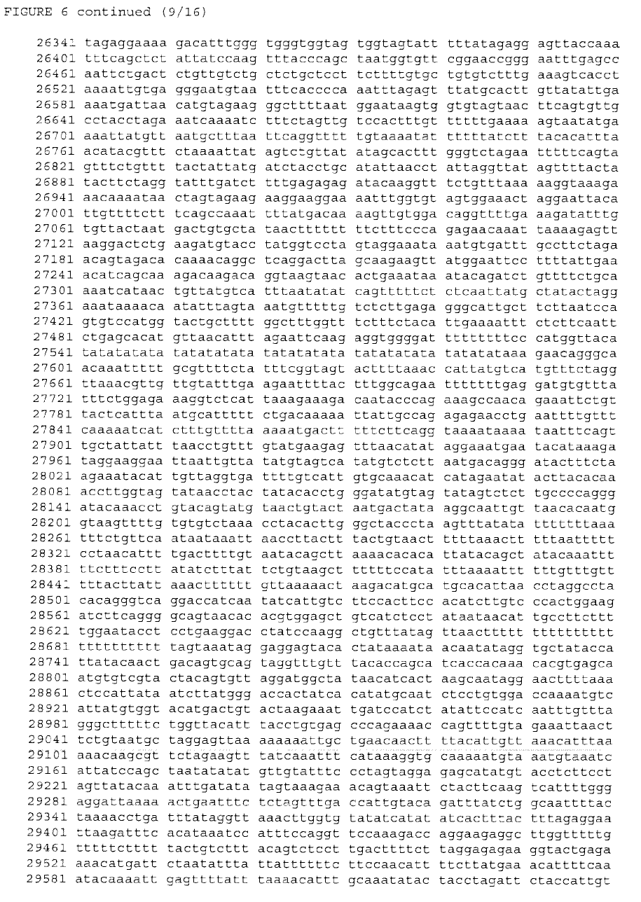

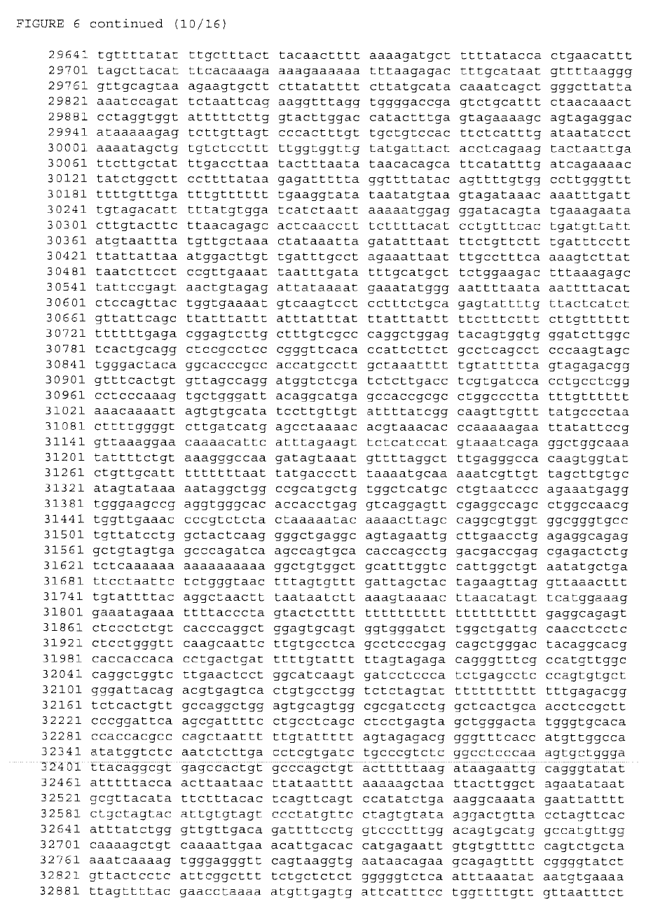

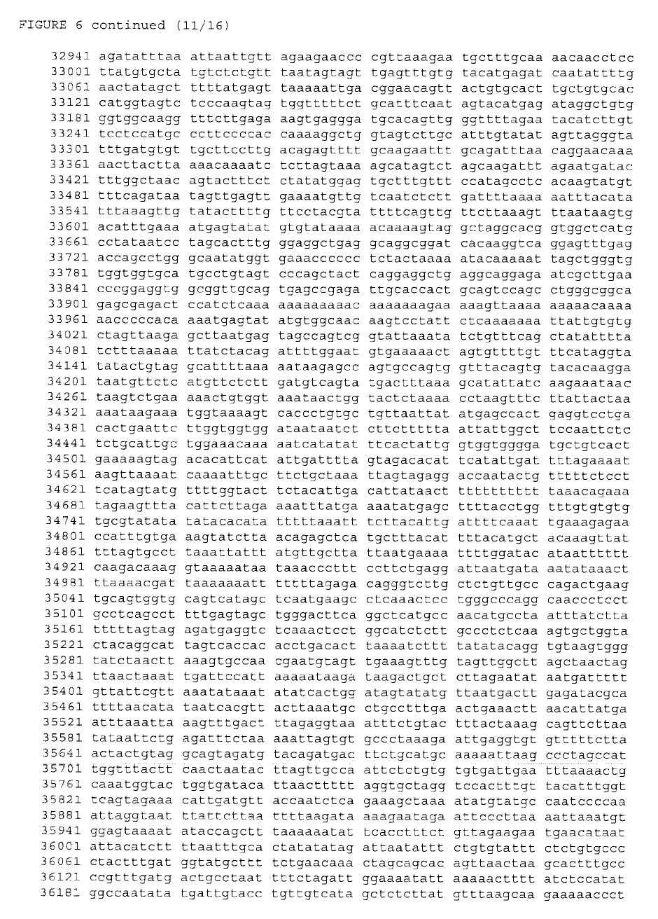

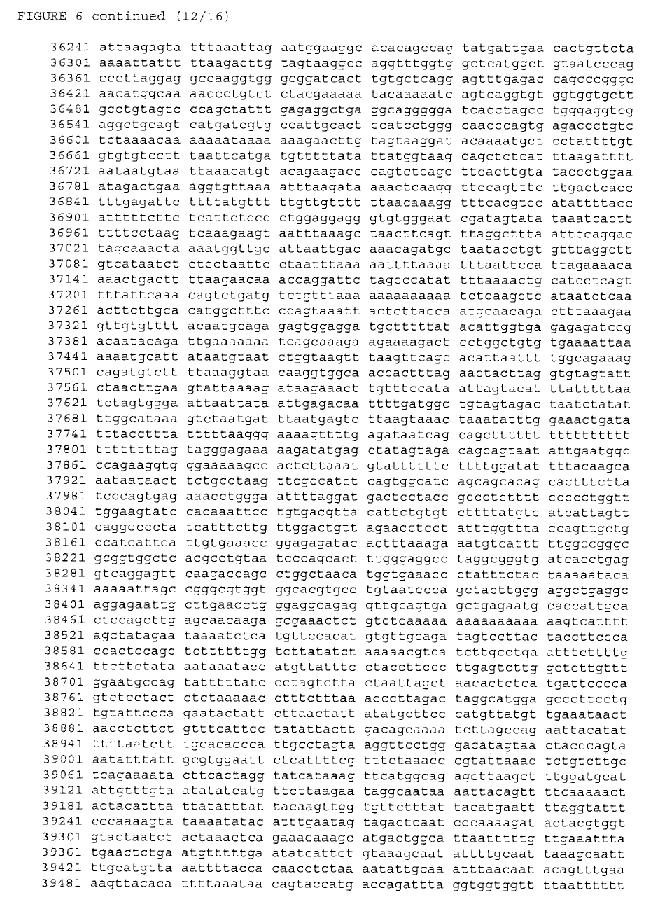

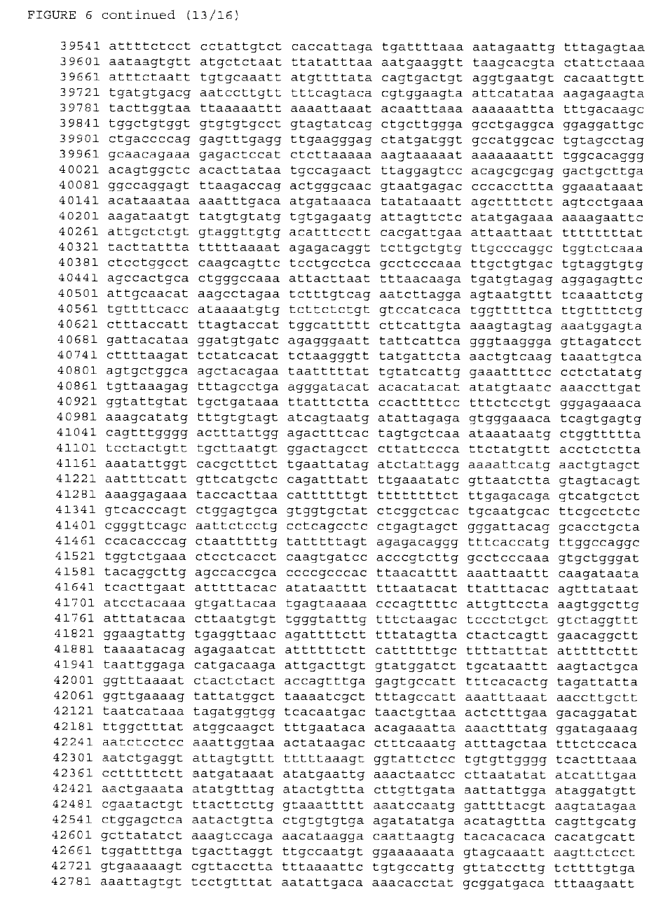

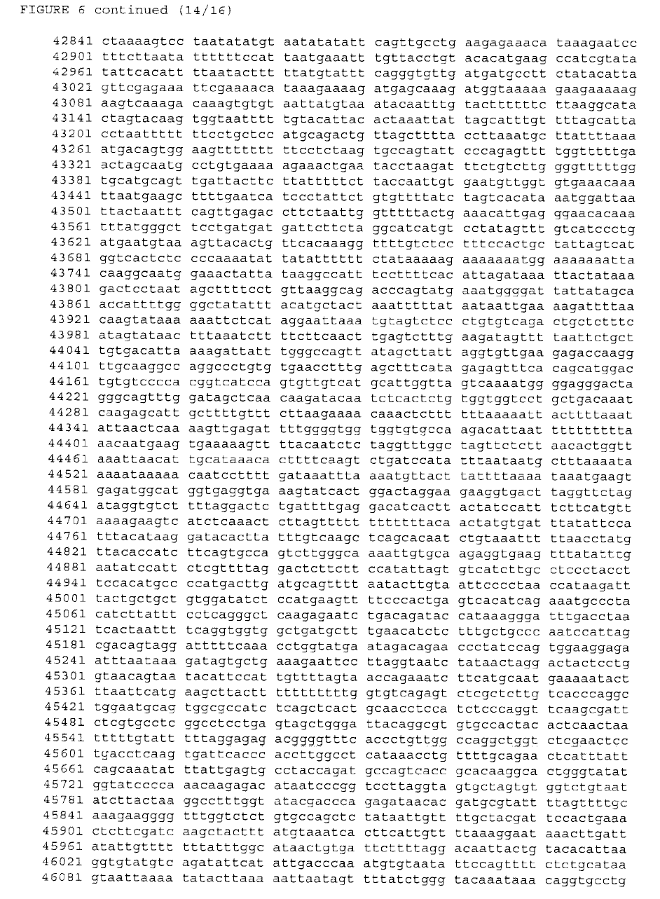

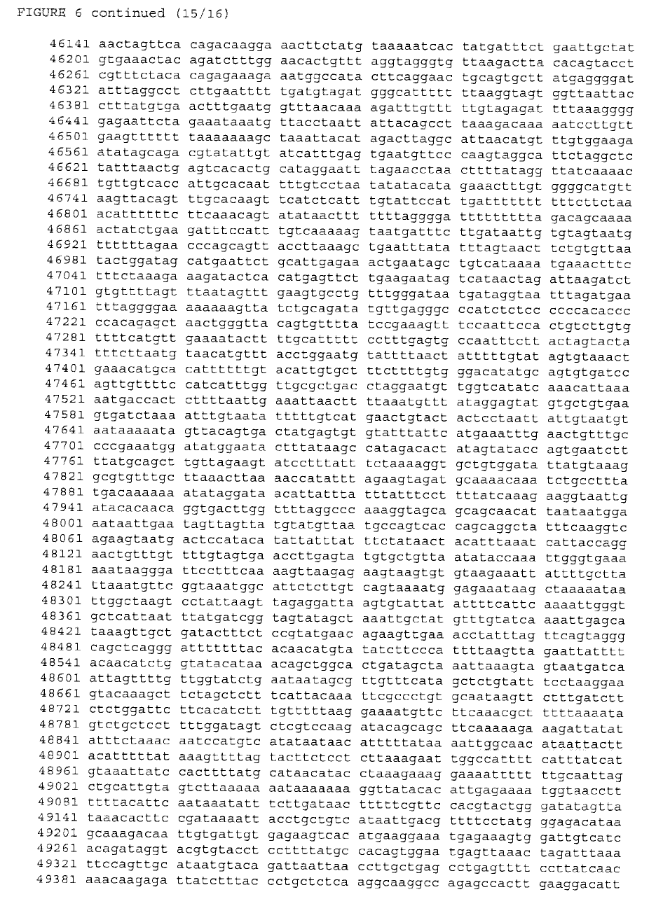

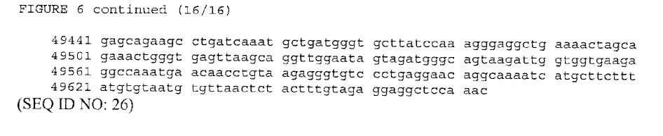

In one embodiment, the internal control may be wild-type K-ras nucleic acid sequence. Full length and partial genomic sequences of human K-ras gene have been reported. Exemplary sequences include but are not limited to NCBI GenBank accession numbers: NG_{—}007524, EU332849, EF685662, EF685661, EF471957, EF471953, CH471094, AC022509, NT_{—}009714, NW_{—}925328, NW_{—}001838052. These sequences are incorporated herein by reference. Exemplary sequence of human K-ras genomic DNA sequence is listed as SEQ ID NO: 26 and shown in FIG. 6. In one embodiment, portion of human K-ras gene may be amplified using the primers SEQ ID NO: 29 and SEQ ID NO: 30. In one embodiment, the oligonucleotide probe to detect a portion of human K-ras gene may be SEQ ID NO: 27. In another embodiment, the oligonucleotide probe to detect a portion of human K-ras gene may be SEQ ID NO: 28.

Detection of Bcl-1 in an acellular body fluid can be used for the diagnosis of mantle cell lymphoma particularly when a poor biopsy or an unusual growth pattern hinders recognition of the disease. The diagnosis of MCL has important implications for prognosis and treatment because the clinical course is aggressive and MCL often responds poorly to conventional B-cell Non-Hodgkin Lymphomas (NHL) therapies (Weisenburger et al. Blood. 1996; 87: 4483-94).

Sample

Sample may be of human or non-human origin. In one embodiment, the sample may be obtained from an individual who is suspected of having a disease, or a genetic abnormality. In another embodiment, sample may be obtained from a healthy individual who is assumed of having no disease, or a genetic abnormality. In preferred embodiments, the sample may be obtained from MCL patients. In some embodiments, the sample may be obtained from an individual diagnosed with myeloma. In other embodiments, the sample may be obtained from an individual diagnosed with B-cell neoplasm such as B-cell chronic lymphocytic leukemia (B-CLL).

In preferred embodiment, an individual's plasma may be used efficiently to detect Bcl-1 nucleic by the methods of the present invention. In another embodiment, IgH/Bcl-1 chromosomal translocation (11;14)(q13;q32) may be detected in plasma. Detection of the chromosomal translocation in plasma of an individual has been found be at least as sensitive if not more so than detecting the same translocation from paired peripheral blood cells of the same individual.

Sample Collection and Preparation

Plasma or Serum Preparation Methods

Methods of plasma and serum preparation are well known in the art. Either “fresh” blood plasma or serum, or frozen (stored) and subsequently thawed plasma or serum may be used. Frozen (stored) plasma or serum should optimally be maintained at storage conditions of −20 to −70 degrees centigrade until thawed and used. “Fresh” plasma or serum should he refrigerated or maintained on ice until used. with nucleic acid (e.g., RNA, DNA or total nucleic acid) extraction being performed as soon as possible. Exemplary methods are described below.

Nucleic Acid Extraction and Amplification

The nucleic acid (DNA or RNA) may be isolated from the sample according to any methods well known to those of skill in the art. If necessary the sample may be collected or concentrated by centrifugation and the like. In some embodiments, nucleic acid may be intact. In another embodiment, nucleic acid may be fragmented (e.g., digested with restriction endonucleases, or by sonication or by applying shearing force by methods known in the art).

Various methods of extraction are suitable for isolating the DNA or RNA. Suitable methods include phenol and chloroform extraction. See Maniatis et al., Molecular Cloning, A Laboratory Manual, 2d, Cold Spring Harbor Laboratory Press, page 16.54 (1989). Numerous commercial kits also yield suitable DNA and RNA including, but not limited to, QIAamp™ mini blood kit, Agencourt Genfind™, Roche Cobas® Roche MagNA Pure® or phenol:chloroform extraction using Eppendorf Phase Lock Gels®, and the NucliSens extraction kit (Biomerieux, Marcy I'Etoile, France). In other methods, mRNA may be extracted using MagNA Pure LC mRNA HS kit and Mag NA Pure LC Instrument (Roche Diagnostics Corporation, Roche Applied Science, Indianapolis, Ind.). Other published protocols and commercial kits are available including, for example, Qiagen products such as the QiaAmp DNA Blood MiniKit (Cat.# 51104, Qiagen, Valencia, Calif.), the QiaAmp RNA Blood MiniKit (Cat.# 52304, Qiagen, Valencia, Calif.); Promega products such as the Wizard Genomic DNA Kit (Cat.# A1620, Promega Corp. Madison, Wis.), Wizard SV Genomic DNA Kit (Cat.# A2360, Promega Corp. Madison, Wis.), the SV Total RNA Kit (Cat.# X3100, Promega Corp. Madison, Wis.), PolyATract System (Cat.# Z5420, Promega Corp. Madison, Wis.), or the PurYield RNA System (Cat.# 23740, Promega Corp. Madison, Wis.).

Removal of DNA from Isolated RNA

Circulating extracellular deoxyribonucleic acid (DNA), including tumor-derived or associated extracellular DNA, is also present in plasma and serum. See, Stroun et al. Oncology. 1989; 46: 318-322. Since this DNA will additionally be extracted to varying degrees during the RNA extraction methods described above, it may be desirable or necessary (depending upon clinical objectives) to further purify the RNA extract and remove trace DNA prior to proceeding to further RNA analysis. This may be accomplished using DNase, for example by the method as described by Rashtchian, A., PCR Methods Applic. 4:S83-S91, (1994), as follows.

Probes

Probes are capable of hybridizing to at least a portion of the target nucleic acid. In some embodiments, the probe can hybridize to a portion of K-ras nucleic acid, or to a portion of Bcl-1 nucleic acid or to a portion of IgH nucleic acid. In some embodiments, the probe can hybridize to a portion of Bcl-1 nucleic acid and or to a portion of IgH nucleic acid.

In some embodiments the probes can be about 10 bases, about 20 bases, about 30 bases, about 40 bases, about 50 bases, about 60 bases, about 75 bases, about 100 bases, about 150 bases, about 200 bases.

In some embodiments, probes can be longer. Longer probes can be from few hundred bases to few million bases. In one embodiment, the nucleic acid probes are derived from one, several or all of the human genomic nucleic acid segments provided in a compendium of bacterial artificial chromosomes (BACs) compiled by The BAC Resource Consortium. These probes are usually referred to in the art by their RPI or CTB clone names, see Cheung et al., Nature 409:953-958, 2001. This compendium contains 7,600 cytogenetically defined landmarks on the draft sequence of the human genome (see McPherson et al., Nature 409:934-41, 2001). These landmarks are large-insert clones mapped to chromosome bands by fluorescence in situ hybridization, each containing a sequence tag that is positioned on the genomic sequence. These clones represent all 24 human chromosomes in about 1 Mb resolution. Sources of BAC genomic collections include the BACPAC Resources Center (CHORI—Children's Hospital Oakland Research Institute), ResGen (Research Genetics through Invitrogen) and The Sanger Center (UK).

Probes consist of a detectable label or a plurality of detectable labels. In one preferred embodiment, the detectable label associated with the probe can generate a detectable signal directly. In another embodiment, the detectable label associated with the probe can be detected indirectly using a reagent, wherein the reagent includes a detectable label, and binds to the label associated with the probe. In one embodiment the reagent includes a detectable label is a labeled antibody. In another embodiment the reagent including a detectable label is a primary antibody/secondary antibody pair, wherein the detectable label may be in the primary antibody, or in the secondary antibody or in both.

In one embodiment, probes are TaqMan® probes, molecular beacons, and Scorpions (e.g., Scorpion™ probes). These types of probes are based on the principle of fluorescence quenching and involve a donor fluorophore and a quenching moiety. The term “fluorophore” as used herein refers to a molecule that absorbs light at a particular wavelength (excitation frequency) and subsequently emits light of a longer wavelength (emission frequency). The term “donor fluorophore” as used herein means a fluorophore that, when in close proximity to a quencher moiety, donates or transfers emission energy to the quencher. As a result of donating energy to the quencher moiety, the donor fluorophore will itself emit less light at a particular emission frequency that it would have in the absence of a closely positioned quencher moiety.

The term “quencher moiety” as used herein means a molecule that, in close proximity to a donor fluorophore, takes up emission energy generated by the donor and either dissipates the energy as heat or emits light of a longer wavelength than the emission wavelength of the donor. In the latter case, the quencher is considered to be an acceptor fluorophore. The quenching moiety can act via proximal (i.e., collisional) quenching or by Förster or fluorescence resonance energy transfer (“FRET”). Quenching by FRET is generally used in TaqMan® probes while proximal quenching is used in molecular beacon and Scorpion™ type probes. Suitable quenchers are selected based on the fluorescence spectrum of the particular fluorophore. Useful quenchers include, for example, the Black Hole™ quenchers BHQ-1, BHQ-2, and BHQ-3 (Biosearch Technologies, Inc.), and the ATTO-series of quenchers (ATTO 540Q, ATTO 580Q, and ATTO 612Q; Atto-Tec GmbH).

With Scorpion primers, sequence-specific priming and PCR product detection is achieved using a single molecule. The Scorpion primer maintains a stem-loop configuration in the unhybridized state. The fluorophore is attached to the 5′ end and is quenched by a moiety coupled to the 3′ end, although in suitable embodiments, this arrangement may be switched The 3′ portion of the stem also contains sequence that is complementary to the extension product of the primer. This sequence is linked to the 5′ end of a specific primer via a non-amplifiable monomer. After extension of the primer moiety, the specific probe sequence is able to bind to its complement within the extended amplicon thus opening up the hairpin loop. This prevents the fluorescence from being quenched and a signal is observed. A specific target is amplified by the reverse primer and the primer portion of the Scorpion primer, resulting in an extension product. A fluorescent signal is generated due to the separation of the fluorophore from the quencher resulting from the binding of the probe element of the Scorpion primer to the extension product.

TaqMan® probes (Heid et al., Genome Res. 1996; 6: 986-994) use the fluorogenic 5′ exonuclease activity of Taq polymerase to measure the amount of target sequences in cDNA samples. TaqMan® probes are oligonucleotides that contain a donor fluorophore usually at or near the 5′ base, and a quenching moiety typically at or near the 3′ base. The quencher moiety may be a dye such as TAMRA or may be a non-fluorescent molecule such as 4-(4-dimethylaminophenylazo) benzoic acid (DABCYL). See Tyagi, et al., 16 Nature Biotechnology 49-53 (1998). When irradiated, the excited fluorescent donor transfers energy to the nearby quenching moiety by FRET rather than fluorescing. Thus, the close proximity of the donor and quencher prevents emission of donor fluorescence while the probe is intact.

TaqMan® probes are designed to anneal to an internal region of a PCR product. When the polymerase (e.g., reverse transcriptase) replicates a template on which a TaqMan® probe is hound, its 5′ exonuclease activity cleaves the probe. This ends the activity of the quencher (no FRET) and the donor fluorophore starts to emit fluorescence which increases in each cycle proportional to the rate of probe cleavage. Accumulation of PCR product is detected by monitoring the increase in fluorescence of the reporter dye (note that primers are not labeled). If the quencher is an acceptor fluorophore, then accumulation of PCR product can be detected by monitoring the decrease in fluorescence of the acceptor fluorophore.

Detectable Label

Detectable label may be associated with a probe and may be used to identify the probe hybridized to a genomic nucleic acid or reference nucleic acid.

Detectable labels include but are not limited to fluorophores, isotopes (e.g. 32P, 33P, 35S, 3H, 14C, 125I, 131I), electron-dense reagents (e.g., gold, silver), nanoparticles, enzymes commonly used in an ELISA (e.g., horseradish peroxidase, beta-galactosidase, luciferase, alkaline phosphatase), chemiluminiscent compound, colorimetric labels (e.g., colloidal gold), magnetic labels (e.g., Dynabeads™), biotin, digoxigenin, haptens, proteins for which antisera or monoclonal antibodies are available, ligands, hormones, oligonucleotides capable of forming a complex with the corresponding oligonucleotide complement.

In some embodiments, the detectable label is a fluorophore. Suitable fluorescent moieties include but are not limited to the following fluorophores working individually or in combination:

4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2′-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120), 7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′,5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl) aminofluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescein (TET); fluorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron® Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (TEXAS RED®); terbium chelate derivatives; N,N,N′,N′-tetramethyl-6-carboxyrhodamine (TAMRA); tetramethyl rhodamine; tetramethyl rhodamine isothiocyanate (TRITC).

DNA Sequencing:

In some embodiments, detection of nucleic acid is by DNA sequencing. Sequencing may be carried out by the dideoxy chain termination method of Sanger et al. (Proc. Natl. Acad. Sci. USA 1977; 74: 5463-5467) with modifications by Zimmermann et al. Nucleic Acids Res. 1990; 18: 1067. Sequencing by dideoxy chain termination method can be performed using Thermo Sequenase (Amersham Pharmacia, Piscataway, N.J.), Sequenase reagents from US Biochemicals or Sequatherm sequencing kit (Epicenter Technologies, Madison, Wis.). Sequencing may also be carried out by the “RR dRhodamine Terminator Cycle Sequencing Kit” from PE Applied Biosystems (product no. 403044, Weiterstadt, Germany), Taq DyeDeoxy™ Terminator Cycle Sequencing kit and method (Perkin-Elmer/Applied Biosystems) in two directions using an Applied Biosystems Model 373A DNA or in the presence of dye terminators CEQ™ Dye Terminator Cycle Sequencing Kit, (Beckman 608000). Alternatively, sequencing can be performed by a method known as Pyrosequencing (Pyrosequencing, Westborough, Mass).

Detection of IgH/Bcl-1 Translocation by Hybridization of a Nucleic Acid Probe

IgH/Bcl-1 translocation may be detected by hybridization of a nucleic acid probe to genomic DNA or to a portion of amplified genomic DNA comprising the translocation. Probes may encompass the junction of IgH/Bcl-1 nucleic acid where a first portion of the probe may be specific for a portion of Bcl-1 nucleic acid and a second portion of the probe may be specific IgH nucleic acid. Exemplary probes for detection of IgH/Bcl-1 translocation with the binding locations for their first and second portions are shown in Table 1 below.

TABLE 1
Exemplary probes for detection of IgH/Bcl-1 translocation

| | First portion of the | Second portion of the |
|---|---|---|
| Sequence encompassing | probe binds to all or | probe binds to all or |
| IgH/Bcl-1 junction | portion of nucleotides | portion of nucleotides |
| SEQ ID NO: 5 | 1-80 | 97-176 |
| SEQ ID NO: 6 | 1-80 | 89-168 |
| SEQ ID NO: 7 | 1-59 | 72-151 |
| SEQ ID NO: 8 | 1-80 | 87-166 |
| SEQ ID NO: 9 | 1-908 | 914-951 |
| SEQ ID NO: 10 | 1-317 | 318-375 |
| SEQ ID NO: 11 | 402 | 403-448 |
| SEQ ID NO: 12 | 1-447 | 448-493 |
| SEQ ID NO: 13 | 1-120 | 121-346 |
| SEQ ID NO: 14 | 1-320 | 321-518 |
| SEQ ID NO: 15 | 1-28 | 42-70 |
| SEQ ID NO: 16 | 1-31 | 50-93 |
| SEQ ID NO: 17 | 1-38 | 47-89 |
| SEQ ID NO: 18 | 1-38 | 52-85 |
| SEQ ID NO: 19 | 1-30 | 36-66 |
| SEQ ID NO: 20 | 1-18 | 19-37 |
| SEQ ID NO: 21 | 1-18 | 19-36 |
| SEQ ID NO: 22 | 1-32 | 35-50 |
| SEQ ID NO: 23 | 1-39 | 74-108 |

Detection of IgH/Bcl-1 Translocation by Comparative Genomic Hybridization

IgH/Bcl-1 chromosomal translocation can he detected by performing an array-based comparative genomic hybridization (CGH) to detect a chromosomal abnormality in a sample, or to diagnose a genetic abnormality in an individual.

Nucleic acids probes can be immobilized to or applied to an array or “biochip”. The term “array” or “microarray” or “biochip” or “chip” as used herein is a plurality of elements arranged onto a defined area of a substrate surface. In practicing the methods of the invention, any known array and/or method of making and using arrays can be incorporated in whole or in part, or variations thereof, as disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5.807,522; 5,800,992; 5,744,305; 5,700.637; 5,556,752; 5,434,049; see also, e.g., WO 99/51773; WO 99/09217; WO 97/46313; WO 96/17958; see also, e.g., Johnston et al. Curr. Biol. 1998 8: R171-R174,; Schummer et al. Biotechniques 1997; 23: 1087-1092; Kern et al. Biotechniques. 1997; 23: 120-124; Solinas-Toldo et al. Genes, Chromosomes & Cancer. 1997; 20: 399-407; Bowtell et al. Nature Genetics. 1999; Supp. 21: 25-32. See also published U.S. Patent Applications Nos. 20010018642; 20010019827; 20010016322; 20010014449; 20010014448; 20010012537; 20010008765.

Arrays are generically a plurality of “target elements” or “spots”, each target element containing a defined amount of one or more biological molecules, e.g., nucleic acid molecules, or probes, immobilized at discrete locations on a substrate surface. In preferred embodiments, the plurality of spots comprises nucleic acid segments, immobilized at preferably at least about 10, at least about 20, at least about 50, at least about 100, at least about 300, or at least about 500 discrete locations on the surface. The plurality may comprise multiple repeats of the same nucleic acid segments to produce, e.g., duplicate spots, triplicate spots, quadruplicate spots, quintuplicate spots, etc.

The resolution of array-based CGH is primarily dependent upon the number, size and map positions of the nucleic acid elements within the array, which are capable of spanning the entire genome. Typically, bacterial artificial chromosomes, or BACs, which can each accommodate on average about 150 kilobases (kb) of cloned genomic DNA, are used in the production of the array.

In a preferred embodiment, the surface comprises an array containing one, several or all of the human genomic nucleic acid segments provided in a compendium of bacterial artificial chromosomes (BACs) compiled by The BAC Resource Consortium, and referred to in the art by their RPI or CTB clone names, see Cheung et al. Nature. 2001; 409: 953-958. This compendium contains 7,600 cytogenetically defined landmarks on the draft sequence of the human genome (see McPherson et al. Nature. 2001; 409: 934-41). These landmarks are large-insert clones mapped to chromosome bands by fluorescence in situ hybridization, each containing a sequence tag that is positioned on the genomic sequence. These clones represent all 24 human chromosomes in about 1 Mb resolution. Sources of BAC genomic collections include the BACPAC Resources Center (CHORI—Children's Hospital Oakland Research Institute), ResGen (Research Genetics through Invitrogen) and The Sanger Center (UK).

Charge-coupled devices, or CCDs, are used in microarray scanning systems, including practicing the methods of the invention. Calculation is based on intensities of red, green and blue light (RGB) as recorded by the separate channels of the camera.

Kits

The present inventions also contemplate diagnostic systems in kit form. A diagnostic system of the present inventions may include a kit which contains, in an amount sufficient for at least one assay, any of the hybridization assay probes, amplification primers, for detecting Bcl-1 nucleic acid and IgH/Bcl-1 translocation in a packaging material. Typically, the kits will also include instructions recorded in a tangible form (e.g., contained on paper or an electronic medium) for using the packaged probes, primers, and/or antibodies in a detection assay for determining the presence or amount of Bcl-1 nucleic acid and IgH/Bcl-1 translocation in a test sample.

The various components of the diagnostic systems may be provided in a variety of forms. For example, the required enzymes, the nucleotide triphosphates, the probes, primers, and/or antibodies may be provided as a lyophilized reagent. These lyophilized reagents may be pre-mixed before lyophilization so that when reconstituted they form a complete mixture with the proper ratio of each of the components ready for use in the assay. In addition, the diagnostic systems of the present inventions may contain a reconstitution reagent for reconstituting the lyophilized reagents of the kit. In preferred kits for amplifying target nucleic acid derived from a MCL patients, the enzymes, nucleotide triphosphates and required cofactors for the enzymes are provided as a single lyophilized reagent that, when reconstituted, forms a proper reagent for use in the present amplification methods.

In one embodiment, the kit may comprise at least three lyophilized oligonucleotides: a primer pair to amplify a portion of Bcl-1 nucleic acid and a portion of nucleic acid comprising IgH/Bcl-1 translocation and a detectably labeled probe capable of hybridizing to the amplicon generated. In some preferred kits, at least three lyophilized oligonucleotides: the detectably labeled probe, and the primer pair for amplification of at least a portion of nucleic acid comprising IgH/Bcl-1 translocation may have sequences of SEQ ID NO: 39, 38, 40 or complements and fragments thereof respectively. In some embodiments, the kit any comprise primers and probes for internal control. In one embodiment, the kit may comprise primers and probes for amplification and detection of human K-ras gene. In one embodiment, the kit may comprise oligonucleotide probes SEQ ID NO: 27 and SEQ ID NO: 28. In another embodiment, the kit may comprise primers for amplifying a portion of K-ras gene: SEQ ID NO: 29 and SEQ ID NO: 30.

Some preferred kits may further comprise to a solid support for anchoring the nucleic acid of interest on the solid support. The target nucleic acid may be anchored to the solid support directly or indirectly through a capture probe anchored to the solid support and capable of hybridizing to the nucleic acid of interest. Examples of such solid support include but are not limited to beads, microparticles (for example, gold and other nano particles), microarray, microwells, multiwell plates. The solid surfaces may comprise a first member of a binding pair and the capture probe or the target nucleic acid may comprise a second member of the binding pair. Binding of the binding pair members will anchor the capture probe or the target nucleic acid to the solid surface. Examples of such binding pairs include but are not limited to biotin/streptavidin, hormone/receptor, ligand/receptor, antigen/antibody.

The versatility of the invention is illustrated by the following Examples which illustrate preferred embodiments of the invention and are not limiting of the claims or specification in any way.

### Example 1

Sample Collection

Blood was collected in EDTA-containing tubes (Becton Dickinson, NJ) from 215 individuals suspected with lymphoid malignancies (based on tissue biopsies and typical lymphoid infiltrate diagnosis) and 195 individuals without lymphoid malignancies. Plasma was separated from blood cells by differential centrifugation at 1000×g for 15 min. Respective blood cells were separated from RBC by differential centrifugation using Puregene® RBC lysis solution (Gentra Systems, MN, USA). The cell pellet was washed with phosphate-buffered saline. Both plasma and cell samples were cryopreserved at −80° C. for future use.

Total DNA from plasma and cell samples were isolated using BioRobot® EZ1 automated nucleic acid purification workstation (QiaGen, CA, USA).

### Example 2

PCR Amplification of Isolated DNA

PCR amplification was performed in triplicate for K-ras gene, IgH gene, IgH/Bcl-1 translocation, TCR-γ, and IgH/Bcl-2 translocation using the primers listed in Table 2. PCR was performed using ABI 7900 detection system and the PCR conditions discussed below. IgH/Bcl-1 translocation was detected by detecting the proximity of a portion of Bcl-1 nucleic acid and a portion of IgH nucleic acid on a single polynucleotide. PCR primers SEQ ID NO: 38 and 40 were used to amplify portions of Bcl-1 and IgH nucleic acids on a single polynucleotide, and a probe (SEQ ID NO: 39) was used to detect the amplified product. For the detection of clonal rearrangement of the IgH gene, FR2a/J_{H} primer pairs (SEQ ID NO's: 31 and 32), FR3/Jh primer pairs and FR3a/CDR3 primer pairs (SEQ ID NO's: 34 and 35) were used. For the detection of clonal rearrangement of TCR-γ primer pairs SEQ ID NO's 36 and 37 were used. In each experiment, amplification of the K-ras gene served as an internal positive control and sterile water served as negative control and used as base line. Amplification of K-ras gene was performed using primer pairs SEQ ID NO's 29 and 30.

TABLE 2
Sequences of primers and probes used in PCR
analysis

| | Primer |
|---|---|
| Internal reference | |
| gene | |
| K-ras-F probe | 5′-FAM-ATGACTGAATATAAACTTGT-3′ |
| | (SEQ ID NO: 27) |
| K-ras-R probe | 5′-FAM-TGGTAGTTGGAGCTGGTGGCGTA- |
| | TAMRA-3′ |
| | (SEQ ID NO: 28) |
| K-ras-F | 5′-GCCTGCTGAAAATGACTGAAT-3′ |
| | (SEQ ID NO: 29) |
| K-ras-R | 5′-GGTCCTGCACCAGTAATATGC-3′ |
| | (SEQ ID NO: 30) |
| IgH chain gene | |
| J_{H}-FAM | 5′-FAM-ACCTGAGGAGACGGTGACC-3′ |
| | (SEQ ID NO: 31) |
| FR2a | 5′-TGGRTCCGMCAGGCYCNGG-3′ |
| | (SEQ ID NO: 32) |
| FR3a | 5′-TGTCGACACGGCYSTGTATTACTG-3′ |
| | (SEQ ID NO: 33) |
| V_{H}-FR3a-FAM | 5′-FAM-ACACGGCCGTGTATTACTG-3′ |
| | (SEQ ID NO: 34) |
| J_{H}-CDR3 | 5′-GTGACCAGGGTNCCTTGGCCCCAG-3′ |
| | (SEQ ID NO 35) |
| TCR-γ chain gene | |
| TcellV-F-FAM | 5′-FAM-CAGGGTTGTGTTGGAATCAGG-3′ |
| | (SEQ ID NO: 36) |
| TcellJ-R | 5′-TGTTCCACTGCCAAAGAGTTTCTT-3′ |
| | (SEQ ID NO: 37) |
| BCL-1 gene | |
| BCL-1 MTC-F | 5′-TGGATAAAGGCGAGGAGCATAA-3′ |
| | (SEQ ID NO: 38) |
| BCL-1 MTC-F probe | 5′-FAM-ACTGCATATTCGGTTAGACTGTGAT |
| | TAGCTTT-TAMRA-3′ |
| | (SEQ ID NO: 39) |
| J_{H}-BCL-1-R | 5′-ACCTGAGGAGACGGTGACC-3′ |
| | (SEQ ID NO: 40) |
| BCL-2 gene | |
| BCL-2 MBR-F | 5′-TTAGAGAGTTGCTTTACGTGGCC-3′ |
| | (SEQ ID NO: 41) |
| BCL-2 MCR-F | 5′-CCTGGCTTCCTTCCCTCTGT-3′ |
| | (SEQ ID NO: 42) |
| BCL-2 MBR probe | 5′-FAM-CAGGAGGGCTCTGGGTGGGTCTGT- |
| | TAMRA-3′ |
| | (SEQ ID NO: 43) |
| BCL-2 MCR probe | 5′-FAM-TGTCCTTCCTTTCCACTCCTCCCCA |
| | GA-TAMRA-3′ |
| | (SEQ ID NO: 44) |
| J_{H}-BCL-2-R | 5′-ACCTGAGGAGACGGTGACC-3′ |
| | (SEQ ID NO: 45) |

PCR Conditions

For IgH gene: 94° C. for 8 min, 52° C. for 20 sec, and 72° C. for 5 min. Cycle repeated for 35 times. For TCR-γ gene: 94° C. for 8 min, 60° C. for 90 sec, 72° C. for 10 min. Cycle repeated for 35 times. For IgH/Bcl-1 translocation: 95° C. for 10 min (1^{st} cycle); 95° C. for 15 sec, 60° C. for 1 min. Cycle repeated 44 times. For IgH/Bcl-2 translocation: 95° C. for 10 min (1^{st} cycle); 95° C. for 15 sec, 60° C. for 1.5 min. Cycle repeated 44 times. The PCR conditions for the internal control gene K-ras were similar to each of the individual set of amplification such as IgH gene, TCR-γ gene, IgH/Bcl-1 translocation or IgH/Bcl-2 translocation.

### Example 3

Analysis of the PCR Amplified Fragments.

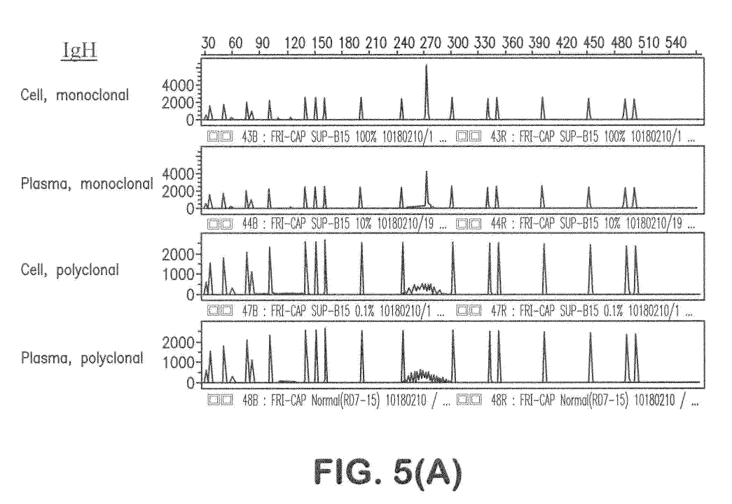

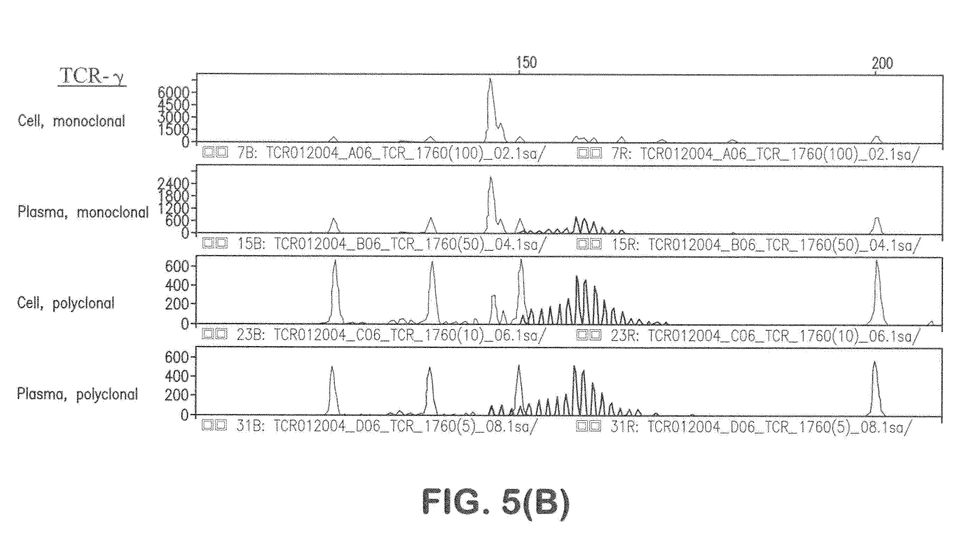

PCR amplified fragments were analyzed by capillary electrophoresis using ABI PRISM® 3100 genetic analyzer (Applied Biosystems, CA, USA). The size of the K-ras amplified product was 108-bp. Predominant amplification product sizes for clonal rearrangement of the IgH gene ranged from 220-310 bp for FR2a/J_{H} primer pairs (SEQ ID NO's: 31 and 32), 70-150 bp for FR3/Jh primer pairs (SEQ ID NO's: 31 and 33) and 50-140 bp for FR3a/CDR3 primer pairs (SEQ ID NO's: 34 and 35) and shown in FIG. 5. The amplification product sizes for clonal rearrangement of TCR-γ ranged from 140-180 bp using primer pairs SEQ ID NO's 36 and 37 and shown in FIG. 5. Predominant amplification product sizes for IgH/Bcl-1 translocation ranged from 200-300 bp using primer pairs SEQ ID NO's 38 and 40.

### Example 4

Sensitivity of Detection of Nucleic Acids Comprising IgH, TCR-γ, IgH/Bcl-1 Translocation and IgH/Bcl-2 Translocation in Acellular Body Fluid

To analyze genomic representation of plasma DNA and its correlation with peripheral blood (PB) cells from the same patient, paired plasma and PB cell samples from patients with suspected lymphoid malignancies were tested for 4 genetic markers: clonal rearrangement of IgH, clonal rearrangement of TCR-γ, IgH/Bcl-1 translocation and IgH/Bcl-2 translocation. PCR amplification followed by capillary electrophoresis was performed on nucleic acids isolated from paired plasma and peripheral blood cells. Results from paired plasma and peripheral blood cells were compared. The results are shown in Table 3 below.

TABLE 3
Correlation between matched plasma and peripheral blood (PB) cell
samples for 4 lymphoid malignancy-specific gene rearrangements

| | Plasma, n (%) | | | | | |
|---|---|---|---|---|---|---|
| | Positive | | Negative | | Total | Concordance, P* |
| PB cells | | | | | | |
| IgH | | | | | | |
| Positive | 17 | (100) | 0 | (0) | 17 | 100% |
| Negative | 0 | (0) | 40 | (100) | 40 | |
| TCR-γ | | | | | | |
| Positive | 17 | (100) | 0 | (0) | 17 | 100% |
| Negative | 0 | (0) | 40 | (100) | 40 | |
| BCL-1/IgH | | | | | | |
| Positive | 17 | (100) | 0 | (0) | 7 | 100% |
| Negative | 0 | (0) | 30 | (100) | 30 | |
| BCL-2/IgH | | | | | | |
| Positive | 17 | (100) | 0 | (0) | 17 | 94%, p < 0.001 |
| Negative | 4 | (7) | 53 | (93) | 57 | |

*All comparisons used Fisher's exact test.
The numbers of healthy subjects included as the control group in IgH, TCR-γ, BCL-1, and BCL-2 studies are 54, 35, 52 and 54, respectively.

A 100% concordance was observed for B-cell clonality between results from PB cells and plasma. 17 of 57 cases were identified as monoclonal and rest were identified as polyclonal population.

For IgH/Bcl-1 translocation, 100% concordance was observed in the results from plasma samples and that of peripheral blood cells of 7 patients tested positive for the translocation. 30 individuals who were tested negative in plasma were was also found to be negative in peripheral blood cells.

A quantitative comparison between paired plasma and peripheral blood cells of patients tested positive to the IgH/Bcl-1 translocation was made. A ratio was determined for the quantitative value of IgH/Bcl-1 obtained by quantitative PCR to the quantitative value of the internal control (K-ras gene) and shown in Table 3 below.

TABLE 3
Quantitative comparison between paired plasma
and blood cell samples of patients with positive
BCL-1 an BCL-2 gene rearrangements*

| | |
|---|---|
| BCL-1 | BCL-2 |

| Sample | Plasma | PB cells | Plasma | PB cells |
|---|---|---|---|---|
| 1 | 1.279 | 0.959 | 0.517 | 0.034 |
| 2 | 2.874 | 2.487 | 30.613 | 18.763 |
| 3 | 0.541 | 0.049 | 1.245 | 0.004 |
| 4 | 0.415 | 0.199 | 8.915 | 0.025 |
| 5 | 17.647 | 13.359 | 0.015 | 0.079 |
| 6 | 4.584 | 3.163 | 0.042 | 0.007 |
| 7 | 6.211 | 9.368 | 0.319 | 0.406 |

*Data is expressed as the ratio of the absolute quantitative value of IgH/BCL-1 by quantitative PCR to the absolute quantitative value of K-ras gene (the internal reference gene).

Unless otherwise defined, all technical and scientific terms used herein have the same meaning as commonly understood by one of ordinary skill in the art to which this invention belongs.

The inventions illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed.

Thus, it should be understood that although the invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

What is claimed is:

1. A method for determining the presence or absence of IgH/Bcl-1 chromosomal translocation in an individual, said method comprising:
a) evaluating nucleic acid from an acellular bodily fluid sample of said individual to determine whether a portion of Bcl-1 nucleic acid is located in close proximity to a portion of IgH nucleic acid on a single polynucleotide; and
b) identifying said individual as having chromosomal translocation of the Bcl-1 nucleic acid when a portion of Bcl-1 nucleic acid is in close proximity to a portion of IgH nucleic acid on a single polynucleotide.

2. The method of claim 1, wherein said acellular body fluid is plasma or serum.

3. The method of claim 1, wherein said portion of IgH nucleic acid comprises an enhancer.

4. The method of claim 1, wherein the nucleic acid evaluated from said individual is genomic DNA or mRNA.

5. The method of claim 1, wherein said method comprises amplifying said nucleic acid using PCR.

6. The method of claim 5, wherein said method comprises using a PCR primer comprising the nucleotide sequence of SEQ ID NO: 38 or a complement thereof.

7. The method of claim 5, wherein said method comprises using a PCR primer comprising the nucleotide sequence of SEQ ID NO: 40 or a complement thereof.

8. The method of claim 5, wherein said PCR further uses a third primer and a fourth primer.

9. The method of claim 5, wherein said method comprises detecting said chromosomal translocation by hybridizing to the amplified nucleic acid a nucleic acid probe encompassing the junction and a first portion of said probe is specific for IgH nucleic acid and a second portion of said probe is specific for Bcl-1 nucleic acid.

10. The method of claim 9, wherein said probe is SEQ ID NO: 39.

11. The method of claim 1, wherein said method comprises determining the presence or absence of said translocation using flow cytometry.

12. The method of claim 1, wherein said method comprises determining the presence or absence of said translocation by determining the nucleotide sequence of said nucleic acid.

13. The method of claim 1, wherein said method comprises determining the presence or absence of said translocation by determining the size of said nucleic acid.

14. The method of claim 13, wherein said determining the size comprises HPLC.

15. The method of claim 13, wherein said determining the size comprises capillary electrophoresis.

16. The method of claim 1, wherein said individual is diagnosed as having mantle cell lymphoma (MCL).

17. The method of claim 1, wherein said individual is diagnosed as having B-cell myeloma.

18. The method of claim 1, further comprising determining the proportion of translocated Bcl-1 genomic nucleic acid relative to control nucleic acid in said acellular body fluid.

19. The method of claim 18, wherein said control nucleic acid is wild-type Bcl-1 genomic nucleic acid without any translocation.

20. The method of claim 18, wherein said control nucleic acid is K-ras gene.

21. A method for diagnosing an individual as having lymphoid malignancy, said method comprising:
a) providing an acellular bodily fluid sample from said individual;
b) evaluating whether a portion of Bcl-1 nucleic acid is located in close proximity a portion of IgH nucleic acid on a single polynucleotide in said acellular body fluid sample; and
c) identifying said individual as having lymphoid malignancy when a portion of Bcl-1 nucleic acid is in close proximity to a portion of IgH nucleic acid on a single polynucleotide.

22. The method of claim 21, wherein said IgH nucleic acid comprises an enhancer.

23. The method of claim 21, wherein said lymphoid malignancy is mantle cell lymphoma (MCL).

24. The method of claim 21, wherein said lymphoid malignancy is B-cell myeloma.

25. The method of claim 21, wherein said acellular body fluid is plasma or serum.

26. A method of determining a prognosis of an individual diagnosed with a lymphoid malignancy, said method comprising determining the presence or absence of IgH/Bcl-1 chromosomal translocation from an acellular bodily fluid of an individual, and identifying the patient as having poor prognosis, wherein the presence of IgH/Bcl-1 chromosomal translocation is indicative of poor prognosis.

27. The method of claim 26, wherein said lymphoid malignancy is mantle cell lymphoma (MCL).

28. The method of claim 26, wherein said lymphoid malignancy is B-cell myeloma.

29. The method of claim 26, wherein said acellular body fluid is plasma or serum.
